SEQUENCE LISTING (86 sequences)

SEQ ID NO: 1 - protein, 6 aa, Artificial
  CDR-L1 Light chain - antibody 1613F12
KSISKY

SEQ ID NO: 2 - protein, 3 aa, Artificial
  CDR-L2 Light chain - antibody 1613F12
SGS

SEQ ID NO: 3 - protein, 9 aa, Artificial
  CDR-L3 Light chain - antibody 1613F12
QQHHEYPLT

SEQ ID NO: 4 - protein, 8 aa, Artificial
  CDR-H1 Light chain - antibody 1613F12
GFNIRDTY

SEQ ID NO: 5 - protein, 8 aa, Artificial
  CDR-H2 Light chain - antibody 1613F12
LDPANGHT

SEQ ID NO: 6 - protein, 17 aa, Artificial
  CDR-H3 Light chain - antibody 1613F12
ARGAYYYGSSGLFYFDY

SEQ ID NO: 7 - protein, 107 aa, Artificial
  Mu. variable domain Light chain  - antibody 1613F12
DVQITQSPSYLATSPGETITINCRASKSISKYLAWYQEKPGKTNKLLIYSGSTLQSGVPSRFSGSGSGTDFTLTISSLEPEDFAMYFCQQHHEYPLTFGAGTELELK

SEQ ID NO: 8 - protein, 124 aa, Artificial
  Mu. variable domain Heavy chain  - antibody 1613F12
EVHLQQSGAELVKPGASVKLSCTASGFNIRDTYIHWVKQRPEQGLEWIGRLDPANGHTKYGPNFQGRATMTSDTSSNTAYLQLSSLTSEDTAVYYCARGAYYYGSSGLFYFDYWGQGTTLSVSS

SEQ ID NO: 9 - DNA, 18 bp, Artificial
  CDR-L1 Light chain - antibody 1613F12
aagagcattagcaaatat

SEQ ID NO: 10 - DNA, 9 bp, Artificial
  CDR-L2 Light chain - antibody 1613F12
tctggatcc

SEQ ID NO: 11 - DNA, 27 bp, Artificial
  CDR-L3 Light chain - antibody 1613F12
caacagcatcatgaatacccgctcacg

SEQ ID NO: 12 - DNA, 24 bp, Artificial
  CDR-H1 Light chain - antibody 1613F12
ggcttcaacattagagacacctat

SEQ ID NO: 13 - DNA, 24 bp, Artificial
  CDR-H2 Light chain - antibody 1613F12
cttgatcctgcgaatggtcatact

SEQ ID NO: 14 - DNA, 51 bp, Artificial
  CDR-H3 Light chain - antibody 1613F12
gctagaggggcctattactacggtagtagtggtctcttctactttgactac

SEQ ID NO: 15 - DNA, 321 bp, Artificial
  Mu. variable domain Light chain  - antibody 1613F12
gatgtccagataacccagtctccatcttatcttgctacatctcctggagaaaccattactattaattgcagggcaagtaagagcattagcaaatatttagcctggtatcaagaaaaacctgggaaaactaataagcttcttatctactctggatccactttgcaatctggagttccatcaaggttcagtggcagtggatctggtacagatttcactctcaccatcagtagcctggagcctgaagattttgcaatgtatttctgtcaacagcatcatgaatacccgctcacgttcggtgctgggaccgagctggagctgaaa

SEQ ID NO: 16 - DNA, 372 bp, Artificial
  Mu. variable domain Heavy chain  - antibody 1613F12
gaggttcacctgcagcagtctggggcagagcttgtgaagccaggggcctcagtcaagttgtcctgcacagcttctggcttcaacattagagacacctatatccattgggtgaaacagaggcctgaacagggcctggagtggattggaaggcttgatcctgcgaatggtcatactaaatatggcccgaacttccagggcagggccactatgacatcagacacatcctccaacacggcctacctgcagctcagcagcctgacatctgaggacactgccgtctattactgtgctagaggggcctattactacggtagtagtggtctcttctactttgactactggggccaaggcaccactctctcagtctcctca

SEQ ID NO: 17 - protein, 107 aa, Artificial
  Hz1613F12 VL - consensus
DIQMTQSPSSLSASVGDRVTITCRASKSISKYLAWYQQKPGKVPKLLIYSGSTLQSGVPSRFSGSGSGTDFTLTISSLQPEDVATYYCQQHHEYPLTFGGGTKVEIK

SEQ ID NO: 18 - protein, 107 aa, Artificial; identical to SEQ ID NO: 17
  Hz1613F12 VL - VL1 (L1)

SEQ ID NO: 19 - protein, 107 aa, Artificial
  Hz1613F12 VL - VL1 I2V (L2)
DVQMTQSPSSLSASVGDRVTITCRASKSISKYLAWYQQKPGKVPKLLIYSGSTLQSGVPSRFSGSGSGTDFTLTISSLQPEDVATYYCQQHHEYPLTFGGGTKVEIK

SEQ ID NO: 20 - protein, 107 aa, Artificial
  Hz1613F12 VL - VL1 M4I (L3)
DIQITQSPSSLSASVGDRVTITCRASKSISKYLAWYQQKPGKVPKLLIYSGSTLQSGVPSRFSGSGSGTDFTLTISSLQPEDVATYYCQQHHEYPLTFGGGTKVEIK

SEQ ID NO: 21 - protein, 107 aa, Artificial
  Hz1613F12 VL - VL2.1 (L4)
DVQITQSPSSLSASVGDRVTITCRASKSISKYLAWYQQKPGKVPKLLIYSGSTLQSGVPSRFSGSGSGTDFTLTISSLQPEDVATYYCQQHHEYPLTFGGGTKVEIK

SEQ ID NO: 22 - protein, 107 aa, Artificial
  Hz1613F12 VL -VL2.1 V49T (L5)
DVQITQSPSSLSASVGDRVTITCRASKSISKYLAWYQQKPGKTPKLLIYSGSTLQSGVPSRFSGSGSGTDFTLTISSLQPEDVATYYCQQHHEYPLTFGGGTKVEIK

SEQ ID NO: 23 - protein, 107 aa, Artificial
  Hz1613F12 VL - VL2.1 P50N (L6)
DVQITQSPSSLSASVGDRVTITCRASKSISKYLAWYQQKPGKVNKLLIYSGSTLQSGVPSRFSGSGSGTDFTLTISSLQPEDVATYYCQQHHEYPLTFGGGTKVEIK

SEQ ID NO: 24 - protein, 107 aa, Artificial
  Hz1613F12 VL - VL2.2 (L7)
DVQITQSPSSLSASVGDRVTINCRASKSISKYLAWYQQKPGKVPKLLIYSGSTLQSGVPSRFSGSGSGTDFTLTISSLQPEDVATYFCQQHHEYPLTFGGGTKVEIK

SEQ ID NO: 25 - protein, 107 aa, Artificial
  Hz1613F12 VL - VL2.2 V49T (L8)
DVQITQSPSSLSASVGDRVTINCRASKSISKYLAWYQQKPGKTPKLLIYSGSTLQSGVPSRFSGSGSGTDFTLTISSLQPEDVATYFCQQHHEYPLTFGGGTKVEIK

SEQ ID NO: 26 - protein, 107 aa, Artificial
  Hz1613F12 VL - VL2.2 P50N (L9)
DVQITQSPSSLSASVGDRVTINCRASKSISKYLAWYQQKPGKVNKLLIYSGSTLQSGVPSRFSGSGSGTDFTLTISSLQPEDVATYFCQQHHEYPLTFGGGTKVEIK

SEQ ID NO: 27 - protein, 107 aa, Artificial
  Hz1613F12 VL - VL2.3 (L10)
DVQITQSPSSLSASVGDRVTINCRASKSISKYLAWYQEKPGKTNKLLIYSGSTLQSGVPSRFSGSGSGTDFTLTISSLQPEDVATYFCQQHHEYPLTFGGGTKVEIK

SEQ ID NO: 28 - protein, 107 aa, Artificial
  Hz1613F12 VL - VL3 (L11)
DVQITQSPSYLAASVGDTITINCRASKSISKYLAWYQEKPGKTNKLLIYSGSTLQSGVPSRFSGSGSGTDFTLTISSLQPEDVATYFCQQHHEYPLTFGAGTELEIK

SEQ ID NO: 29 - protein, 124 aa, Artificial
  Hz1613F12 VH - consensus
QVQLVQSGAEVKKPGASVKVSCKASGFNIRDTYMHWVRQAPGQGLEWMGWLDPANGHTNYAQKFQGRVTMTRDTSISTAYMELSRLRSDDTAVYYCARGAYYYGSSGLFYFDYWGQGTLVTVSS

SEQ ID NO: 30 - protein, 124 aa, Artificial; identical to SEQ ID NO: 29
  Hz1613F12 VH - VH1 (H1)

SEQ ID NO: 31 - protein, 124 aa, Artificial
  Hz1613F12 VH - VH1 M39I (H2)
QVQLVQSGAEVKKPGASVKVSCKASGFNIRDTYIHWVRQAPGQGLEWMGWLDPANGHTNYAQKFQGRVTMTRDTSISTAYMELSRLRSDDTAVYYCARGAYYYGSSGLFYFDYWGQGTLVTVSS

SEQ ID NO: 32 - protein, 124 aa, Artificial
  Hz1613F12 VH - VH1 W55R N66K (H3)
QVQLVQSGAEVKKPGASVKVSCKASGFNIRDTYMHWVRQAPGQGLEWMGRLDPANGHTKYAQKFQGRVTMTRDTSISTAYMELSRLRSDDTAVYYCARGAYYYGSSGLFYFDYWGQGTLVTVSS

SEQ ID NO: 33 - protein, 124 aa, Artificial
  Hz1613F12 VH - VH1 I84S (H4)
QVQLVQSGAEVKKPGASVKVSCKASGFNIRDTYMHWVRQAPGQGLEWMGWLDPANGHTNYAQKFQGRVTMTRDTSSSTAYMELSRLRSDDTAVYYCARGAYYYGSSGLFYFDYWGQGTLVTVSS

SEQ ID NO: 34 - protein, 124 aa, Artificial
  Hz1613F12 VH - VH1 S85N (H5)
QVQLVQSGAEVKKPGASVKVSCKASGFNIRDTYMHWVRQAPGQGLEWMGWLDPANGHTNYAQKFQGRVTMTRDTSINTAYMELSRLRSDDTAVYYCARGAYYYGSSGLFYFDYWGQGTLVTVSS

SEQ ID NO: 35 - protein, 124 aa, Artificial
  Hz1613F12 VH - VH1 I84N S85N (H6)
QVQLVQSGAEVKKPGASVKVSCKASGFNIRDTYMHWVRQAPGQGLEWMGWLDPANGHTNYAQKFQGRVTMTRDTSSNTAYMELSRLRSDDTAVYYCARGAYYYGSSGLFYFDYWGQGTLVTVSS

SEQ ID NO: 36 - protein, 124 aa, Artificial
  Hz1613F12 VH - VH2.1 (H7)
QVQLVQSGAEVKKPGASVKVSCKASGFNIRDTYIHWVRQAPGQGLEWMGWLDPANGHTNYAQKFQGRVTMTRDTSSNTAYMELSRLRSDDTAVYYCARGAYYYGSSGLFYFDYWGQGTLVTVSS

SEQ ID NO: 37 - protein, 124 aa, Artificial
  Hz1613F12 VH - VH2.1 Q3H (H8)
QVHLVQSGAEVKKPGASVKVSCKASGFNIRDTYIHWVRQAPGQGLEWMGWLDPANGHTNYAQKFQGRVTMTRDTSSNTAYMELSRLRSDDTAVYYCARGAYYYGSSGLFYFDYWGQGTLVTVSS

SEQ ID NO: 38 - protein, 124 aa, Artificial
  Hz1613F12 VH - VH2.1 W55R (H9)
QVQLVQSGAEVKKPGASVKVSCKASGFNIRDTYIHWVRQAPGQGLEWMGRLDPANGHTNYAQKFQGRVTMTRDTSSNTAYMELSRLRSDDTAVYYCARGAYYYGSSGLFYFDYWGQGTLVTVSS

SEQ ID NO: 39 - protein, 124 aa, Artificial
  Hz1613F12 VH - VH2.1 N66K (H10)
QVQLVQSGAEVKKPGASVKVSCKASGFNIRDTYIHWVRQAPGQGLEWMGWLDPANGHTKYAQKFQGRVTMTRDTSSNTAYMELSRLRSDDTAVYYCARGAYYYGSSGLFYFDYWGQGTLVTVSS

SEQ ID NO: 40 - protein, 124 aa, Artificial
  Hz1613F12 VH - VH2.1 W55R N66K (H11)
QVQLVQSGAEVKKPGASVKVSCKASGFNIRDTYIHWVRQAPGQGLEWMGRLDPANGHTKYAQKFQGRVTMTRDTSSNTAYMELSRLRSDDTAVYYCARGAYYYGSSGLFYFDYWGQGTLVTVSS

SEQ ID NO: 41 - protein, 124 aa, Artificial
  Hz1613F12 VH - VH2.1 R80S (H12)
QVQLVQSGAEVKKPGASVKVSCKASGFNIRDTYIHWVRQAPGQGLEWMGWLDPANGHTNYAQKFQGRVTMTSDTSSNTAYMELSRLRSDDTAVYYCARGAYYYGSSGLFYFDYWGQGTLVTVSS

SEQ ID NO: 42 - protein, 124 aa, Artificial
  Hz1613F12 VH - VH2.1 N66K R80S (H13)
QVQLVQSGAEVKKPGASVKVSCKASGFNIRDTYIHWVRQAPGQGLEWMGWLDPANGHTKYAQKFQGRVTMTSDTSSNTAYMELSRLRSDDTAVYYCARGAYYYGSSGLFYFDYWGQGTLVTVSS

SEQ ID NO: 43 - protein, 124 aa, Artificial
  Hz1613F12 VH - VH2.2 (H14)
QVHLVQSGAEVKKPGASVKVSCKASGFNIRDTYIHWVRQAPGQGLEWMGWLDPANGHTKYAQKFQGRVTMTSDTSSNTAYMELSRLRSDDTAVYYCARGAYYYGSSGLFYFDYWGQGTLVTVSS

SEQ ID NO: 44 - protein, 124 aa, Artificial
  Hz1613F12 VH - VH2.2 M89L (H15)
QVHLVQSGAEVKKPGASVKVSCKASGFNIRDTYIHWVRQAPGQGLEWMGWLDPANGHTKYAQKFQGRVTMTSDTSSNTAYLELSRLRSDDTAVYYCARGAYYYGSSGLFYFDYWGQGTLVTVSS

SEQ ID NO: 45 - protein, 124 aa, Artificial
  Hz1613F12 VH - VH2.3 (H16)
QVQLQQSGAEVKKPGASVKLSCTASGFNIRDTYIHWVRQAPGQGLEWMGWLDPANGHTKYAQKFQGRVTMTSDTSSNTAYMELSRLRSDDTAVYYCARGAYYYGSSGLFYFDYWGQGTLVTVSS

SEQ ID NO: 46 - protein, 124 aa, Artificial
  Hz1613F12 VH - VH2.3 W55R (H17)
QVQLQQSGAEVKKPGASVKLSCTASGFNIRDTYIHWVRQAPGQGLEWMGRLDPANGHTKYAQKFQGRVTMTSDTSSNTAYMELSRLRSDDTAVYYCARGAYYYGSSGLFYFDYWGQGTLVTVSS

SEQ ID NO: 47 - protein, 124 aa, Artificial
  Hz1613F12 VH - VH2.3 Q3H W55R (H18)
QVHLQQSGAEVKKPGASVKLSCTASGFNIRDTYIHWVRQAPGQGLEWMGRLDPANGHTKYAQKFQGRVTMTSDTSSNTAYMELSRLRSDDTAVYYCARGAYYYGSSGLFYFDYWGQGTLVTVSS

SEQ ID NO: 48 - protein, 124 aa, Artificial
  Hz1613F12 VH - VH2.4 (H19)
QVQLQQSGAEVKKPGASVKLSCTASGFNIRDTYIHWVRQAPGQGLEWIGRLDPANGHTKYAQKFQGRVTMTSDTSSNTAYLELSRLRSDDTAVYYCARGAYYYGSSGLFYFDYWGQGTLVTVSS

SEQ ID NO: 49 - protein, 124 aa, Artificial
  Hz1613F12 VH - VH3 (H20)
EVHLQQSGAELVKPGASVKLSCTASGFNIRDTYIHWVKQAPGQGLEWIGRLDPANGHTKYGQKFQGRVTMTSDTSSNTAYLQLSRLRSDDTAVYYCARGAYYYGSSGLFYFDYWGQGTLVTVSS

SEQ ID NO: 50 - DNA, 321 bp, Artificial
  Hz1613F12 VL - VL1 (L1)
gatattcaaatgacacagtctccaagctccctttctgcatcagtgggcgatagagtaaccatcacttgccgcgccagcaaaagcatctccaaatacctcgcttggtaccagcagaagccaggcaaggtccctaaactgctcatttattccggcagtaccctgcagtctggggtgccttctcggttcagtggaagtggctccggtaccgactttaccctgactataagctcactccagcccgaggacgtcgctacatattactgtcagcagcaccatgaatatcctctgacattcggtggaggaaccaaggtggagatcaag

SEQ ID NO: 51 - DNA, 321 bp, Artificial
  Hz1613F12 VL - VL1 I2V (L2)
gacgttcaaatgacacagtcccctagttccctctctgcttccgtgggcgatagggtgacaattacttgcagagccagtaagtcaatttcaaagtatcttgcttggtaccagcagaaaccaggcaaagtgcccaagctgcttatctacagtggtagcacacttcaatctggagtcccttctcgattcagcggaagcggctccgggaccgacttcactctgactatcagctctctgcagccagaggatgtcgccacctactattgccagcagcaccatgagtatcccctcacctttggaggtggcaccaaagtggaaatcaag

SEQ ID NO: 52 - DNA, 321 bp, Artificial
  Hz1613F12 VL - VL1 M4I (L3)
gacatccaaatcacacagtcccctagttccctctctgcttccgtgggcgatagggtgacaattacttgcagagccagtaagtcaatttcaaagtatcttgcttggtaccagcagaaaccaggcaaagtgcccaagctgcttatctacagtggtagcacacttcaatctggagtcccttctcgattcagcggaagcggctccgggaccgacttcactctgactatcagctctctgcagccagaggatgtcgccacctactattgccagcagcaccatgagtatcccctcacctttggaggtggcaccaaagtggaaatcaag

SEQ ID NO: 53 - DNA, 321 bp, Artificial
  Hz1613F12 VL - VL2.1 (L4)
gacgtgcaaatcacacagtcccctagttccctctctgcttccgtgggcgatagggtgacaattacttgcagagccagtaagtcaatttcaaagtatcttgcttggtaccagcagaaaccaggcaaagtgcccaagctgcttatctacagtggtagcacacttcaatctggagtcccttctcgattcagcggaagcggctccgggaccgacttcactctgactatcagctctctgcagccagaggatgtcgccacctactattgccagcagcaccatgagtatcccctcacctttggaggtggcaccaaagtggaaatcaag

SEQ ID NO: 54 - DNA, 321 bp, Artificial
  Hz1613F12 VL - VL2.1 V49T (L5)
gacgtgcaaatcacacagtcccctagttccctctctgcttccgtgggcgatagggtgacaattacttgcagagccagtaagtcaatttcaaagtatcttgcttggtaccagcagaaaccaggcaaaacccccaagctgcttatctacagtggtagcacacttcaatctggagtcccttctcgattcagcggaagcggctccgggaccgacttcactctgactatcagctctctgcagccagaggatgtcgccacctactattgccagcagcaccatgagtatcccctcacctttggaggtggcaccaaagtggaaatcaag

SEQ ID NO: 55 - DNA, 321 bp, Artificial
  Hz1613F12 VL - VL2.1 P50N (L6)
gacgtgcaaatcacacagtcccctagttccctctctgcttccgtgggcgatagggtgacaattacttgcagagccagtaagtcaatttcaaagtatcttgcttggtaccagcagaaaccaggcaaagtgaacaagctgcttatctacagtggtagcacacttcaatctggagtcccttctcgattcagcggaagcggctccgggaccgacttcactctgactatcagctctctgcagccagaggatgtcgccacctactattgccagcagcaccatgagtatcccctcacctttggaggtggcaccaaagtggaaatcaag

SEQ ID NO: 56 - DNA, 321 bp, Artificial
  Hz1613F12 VL - VL2.2 (L7)
gatgtgcaaattacacagtctccaagctccctttctgcatcagtgggcgatagagtaaccatcaactgccgcgccagcaaaagcatctccaaatacctcgcttggtaccagcagaagccaggcaaggtccctaaactgctcatttattccggcagtaccctgcagtctggggtgccttctcggttcagtggaagtggctccggtaccgactttaccctgactataagctcactccagcccgaggacgtcgctacatatttttgtcagcagcaccatgaatatcctctgacattcggtggaggaaccaaggtggagatcaag

SEQ ID NO: 57 - DNA, 321 bp, Artificial
  Hz1613F12 VL - VL2.2 V49T (L8)
gacgtgcaaatcacacagtcccctagttccctctctgcttccgtgggcgatagggtgacaattaactgcagagccagtaagtcaatttcaaagtatcttgcttggtaccagcagaaaccaggcaaaacccccaagctgcttatctacagtggtagcacacttcaatctggagtcccttctcgattcagcggaagcggctccgggaccgacttcactctgactatcagctctctgcagccagaggatgtcgccacctacttctgccagcagcaccatgagtaccccctcacctttggaggtggcaccaaagtggaaatcaag

SEQ ID NO: 58 - DNA, 321 bp, Artificial
  Hz1613F12 VL - VL2.2 P50N (L9)
gacgtgcaaatcacacagtcccctagttccctctctgcttccgtgggcgatagggtgacaattaactgcagagccagtaagtcaatttcaaagtatcttgcttggtaccagcagaaaccaggcaaagtgaacaagctgcttatctacagtggtagcacacttcaatctggagtcccttctcgattcagcggaagcggctccgggaccgacttcactctgactatcagctctctgcagccagaggatgtcgccacctacttctgccagcagcaccatgagtaccccctcacctttggaggtggcaccaaagtggaaatcaag

SEQ ID NO: 59 - DNA, 321 bp, Artificial
  Hz1613F12 VL - VL2.3 (L10)
gacgtgcaaatcacacagtcccctagttccctctctgcttccgtgggcgatagggtgacaattaactgcagagccagtaagtcaatttcaaagtatcttgcttggtaccaggagaaaccaggcaaaaccaacaagctgcttatctacagtggtagcacacttcaatctggagtcccttctcgattcagcggaagcggctccgggaccgacttcactctgactatcagctctctgcagccagaggatgtcgccacctacttctgccagcagcaccatgagtaccccctcacctttggaggtggcaccaaagtggaaatcaag

SEQ ID NO: 60 - DNA, 321 bp, Artificial
  Hz1613F12 VL - VL3 (L11)
gacgtccagatcacacagtctccttcctatctggccgcctctgtgggcgataccattaccataaactgcagggcttcaaagagcatcagcaagtacctggcatggtatcaggagaagcccgggaaaaccaataagctcctgatctactccggctctactttgcagtccggagtgcccagccggttttcaggcagtggtagtggaactgactttactctgaccattagctctctgcaacccgaagacgtagctacatacttctgtcagcagcaccatgaatatccactgaccttcggtgccgggacagagctggagatcaaa

SEQ ID NO: 61 - DNA, 372 bp, Artificial
  Hz1613F12 VH - VH1 (H1)
caggtgcagctggtgcagagtggtgctgaggtgaaaaagcccggagcctctgtcaaagtctcttgtaaggcatccgggtttaacatccgggacacatacatgcactgggttaggcaggctccaggccagggtctggaatggatgggatggcttgaccctgctaacggccacactaattacgcccaaaagtttcaggggcgcgtaaccatgaccagagatactagcatatccactgcatacatggagctgagccgactccgtagcgacgataccgccgtgtattattgcgcaaggggagcctattactacggcagtagcggactcttctacttcgactattgggggcaaggcaccctggtcacagtttcatca

SEQ ID NO: 62 - DNA, 372 bp, Artificial
  Hz1613F12 VH - VH1 M39I (H2)
caggttcagctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggctggctggacccagcaaacggccacacaaattacgctcagaaattccaggggagagtcaccatgacccgggacacctcaatctccactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 63 - DNA, 372 bp, Artificial
  Hz1613F12 VH - VH1 W55R N66K (H3)
caggttcagctcgttcagagtggcgccgaggtgaagaagcccggtgctagtgtcaaggtgtcctgcaaagccagcgggttcaacatacgggatacctacatgcactgggtgaggcaagcccctggtcaaggactggaatggatgggcaggctggacccagcaaacggccacacaaaatacgctcagaaattccaggggagagtcaccatgacccgggacacctcaatctccactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 64 - DNA, 372 bp, Artificial
  Hz1613F12 VH - VH1 I84S (H4)
caggttcagctcgttcagagtggcgccgaggtgaagaagcccggtgctagtgtcaaggtgtcctgcaaagccagcgggttcaacatacgggatacctacatgcactgggtgaggcaagcccctggtcaaggactggaatggatgggctggctggacccagcaaacggccacacaaattacgctcagaaattccaggggagagtcaccatgacccgggacacctcaagctccactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 65 - DNA, 372 bp, Artificial
  Hz1613F12 VH - VH1 S85N (H5)
caggttcagctcgttcagagtggcgccgaggtgaagaagcccggtgctagtgtcaaggtgtcctgcaaagccagcgggttcaacatacgggatacctacatgcactgggtgaggcaagcccctggtcaaggactggaatggatgggctggctggacccagcaaacggccacacaaattacgctcagaaattccaggggagagtcaccatgacccgggacacctcaatcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 66 - DNA, 372 bp, Artificial
  Hz1613F12 VH - VH1 I84N S85N (H6)
caggttcagctcgttcagagtggcgccgaggtgaagaagcccggtgctagtgtcaaggtgtcctgcaaagccagcgggttcaacatacgggatacctacatgcactgggtgaggcaagcccctggtcaaggactggaatggatgggctggctggacccagcaaacggccacacaaattacgctcagaaattccaggggagagtcaccatgacccgggacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 67 - DNA, 372 bp, Artificial
  Hz1613F12 VH - VH2.1 (H7)
caggtgcagctggtgcagagtggtgctgaggtgaaaaagcccggagcctctgtcaaagtctcttgtaaggcatccgggtttaacatccgggacacatacatacactgggttaggcaggctccaggccagggtctggaatggatgggatggcttgaccctgctaacggccacactaattacgcccaaaagtttcaggggcgcgtaaccatgaccagagatactagctccaatactgcatacatggagctgagccgactccgtagcgacgataccgccgtgtattattgcgcaaggggagcctattactacggcagtagcggactcttctacttcgactattgggggcaaggcaccctggtcacagtttcatca

SEQ ID NO: 68 - DNA, 372 bp, Artificial
  Hz1613F12 VH - VH2.1 Q3H (H8)
caggttcacctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggctggctggacccagcaaacggccacacaaattacgctcagaaattccaggggagagtcaccatgacccgggacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 69 - DNA, 372 bp, Artificial
  Hz1613F12 VH - VH2.1 W55R (H9)
caggttcagctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggcaggctggacccagcaaacggccacacaaattacgctcagaaattccaggggagagtcaccatgacccgggacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 70 - DNA, 372 bp, Artificial
  Hz1613F12 VH - VH2.1 N66K (H10)
caggttcagctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggctggctggacccagcaaacggccacacaaagtacgctcagaaattccaggggagagtcaccatgacccgggacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 71 - DNA, 372 bp, Artificial
  Hz1613F12 VH - VH2.1 W55R N66K (H11)
caggttcagctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggcaggctggacccagcaaacggccacacaaagtacgctcagaaattccaggggagagtcaccatgacccgggacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 72 - DNA, 372 bp, Artificial
  Hz1613F12 VH - VH2.1 R80S (H12)
caggttcagctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggctggctggacccagcaaacggccacacaaattacgctcagaaattccaggggagagtcaccatgaccagcgacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 73 - DNA, 372 bp, Artificial
  Hz1613F12 VH - VH2.1 N66K R80S (H13)
caggttcagctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggctggctggacccagcaaacggccacacaaagtacgctcagaaattccaggggagagtcaccatgaccagcgacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 74 - DNA, 372 bp, Artificial
  Hz1613F12 VH - VH2.2 (H14)
caggtgcatctggtgcagagtggtgctgaggtgaaaaagcccggagcctctgtcaaagtctcttgtaaggcatccgggtttaacatccgggacacatacatacactgggttaggcaggctccaggccagggtctggaatggatgggatggcttgaccctgctaacggccacactaagtacgcccaaaagtttcaggggcgcgtaaccatgacctctgatactagctccaatactgcatacatggagctgagccgactccgtagcgacgataccgccgtgtattattgcgcaaggggagcctattactacggcagtagcggactcttctacttcgactattgggggcaaggcaccctggtcacagtttcatca

SEQ ID NO: 75 - DNA, 372 bp, Artificial
  Hz1613F12 VH - VH2.2 M89L (H15)
caggttcacctcgttcagagtggcgccgaggtgaagaagcccggtgctagtgtcaaggtgtcctgcaaagccagcgggttcaacatacgggatacctacattcactgggtgaggcaagcccctggtcaaggactggaatggatgggctggctggacccagcaaacggccacacaaagtacgctcagaaattccaggggagagtcaccatgaccagcgacacctcaagcaacactgcatacctggagctttctcgcttgaggagtgatgacacagctgtgtattattgtgccagaggagcatactattatggatcttccggcctgttctactttgactactgggggcagggaaccttggtcacagtgagctca

SEQ ID NO: 76 - DNA, 372 bp, Artificial
  Hz1613F12 VH - VH2.3 (H16)
caggttcagctccagcagagtggcgccgaggtgaagaagcccggtgctagtgtcaagctgtcctgcaccgccagcgggttcaacatacgggatacctacattcactgggtgaggcaagcccctggtcaaggactggaatggatgggctggctggacccagcaaacggccacacaaagtacgctcagaaattccaggggagagtcaccatgaccagcgacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 77 - DNA, 372 bp, Artificial
  Hz1613F12 VH - VH2.3 W55R (H17)
caggttcagctccagcagagtggcgccgaggtgaagaagcccggtgctagtgtcaagctgtcctgcaccgccagcgggttcaacatacgggatacctacattcactgggtgaggcaagcccctggtcaaggactggaatggatgggcaggctggacccagcaaacggccacacaaagtacgctcagaaattccaggggagagtcaccatgaccagcgacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 78 - DNA, 372 bp, Artificial
  Hz1613F12 VH - VH2.3 Q3H W55R (H18)
caggttcacctccagcagagtggcgccgaggtgaagaagcccggtgctagtgtcaagctgtcctgcaccgccagcgggttcaacatacgggatacctacattcactgggtgaggcaagcccctggtcaaggactggaatggatgggcaggctggacccagcaaacggccacacaaagtacgctcagaaattccaggggagagtcaccatgaccagcgacacctcaagcaacactgcatacatggagctttctcgcttgaggagtgatgacacagctgtgtattattgtgccagaggagcatactattatggatcttccggcctgttctactttgactactgggggcagggaaccttggtcacagtgagctca

SEQ ID NO: 79 - DNA, 372 bp, Artificial
  Hz1613F12 VH - VH2.4 (H19)
caggttcagctccagcagagtggcgccgaggtgaagaagcccggtgctagtgtcaagctgtcctgcaccgccagcgggttcaacatacgggatacctacattcactgggtgaggcaagcccctggtcaaggactggaatggatcggcaggctggacccagcaaacggccacacaaagtacgctcagaaattccaggggagagtcaccatgaccagcgacacctcaagcaacactgcatacctggagctttctcgcttgaggagtgatgacacagctgtgtattattgtgccagaggagcatactattatggatcttccggcctgttctactttgactactgggggcagggaaccttggtcacagtgagctca

SEQ ID NO: 80 - DNA, 372 bp, Artificial
  Hz1613F12 VH - VH3 (H20)
gaagttcacttgcagcagtcaggcgccgagcttgtgaaacctggagccagcgtcaaactgtcctgtaccgctagtggattcaatattcgggacacctatatccactgggtaaagcaagcaccagggcagggattggagtggatcggacgcctggatcccgccaacggtcatactaagtacggtcagaagttccaagggagggtgacaatgacctctgataccagctccaacaccgcatatctgcagctgagccgtcttagatctgacgacacagctgtctactattgcgctaggggcgcctactactacgggtccagtggtctgttttacttcgattattggggccagggcactctcgtgacagtgtcaagt

SEQ ID NO: 81 - protein, 107 aa, Artificial; identical to SEQ ID NO: 19
  Hz1613F12 VL - Consensus 2

SEQ ID NO: 82 - protein, 124 aa, Artificial; identical to SEQ ID NO: 32
  Hz1613F12 VH - Consensus 2

SEQ ID NO: 83 - protein, 894 aa, Artificial
  Protein Axl (with peptide signal)
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 84 - protein, 869 aa, Artificial
  Protein Axl (without peptide signal)
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 85 - protein, 451 aa, Artificial
  Extracellular domain of the protein Axl (with the peptide signal)
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWW

SEQ ID NO: 86 - protein, 426 aa, Artificial
  Extracellular domain of the protein Axl (without the peptide signal)
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWW

(19) United States
(12) Patent Application Publication
(10) Pub. No.: US 2016/0106861 A1
(43) Pub. Date: Apr. 21, 2016

(54) AXL ANTIBODY-DRUG CONJUGATE AND ITS USE FOR THE TREATMENT OF CANCER

(71) Applicants: PIERRE FABRE MEDICAMENT, Boulogne-billancourt (FR); SPIROGEN SARL, St-Légier-Chiésaz (CH)

(72) Inventors: Charlotte BEAU-LARVOR, Jonzier Epagny (FR); Liliane GOETSCH, Ayze (FR); Philip Wilson HOWARD, Londres (GB)

(73) Assignees: SPIROGEN SARL, St-Légier-Chiésaz (CH); PIERRE FABRE MEDICAMENT, Boulogne-billancourt (FR)

(21) Appl. No.: 14/786,859

(22) PCT Filed: Apr. 28, 2014
(86) PCT No.: PCT/EP2014/058560

§ 371 Date: Oct. 23, 2015

(30) Foreign Application Priority Data
Apr. 26, 2013 (EP) 13305549.1

(51) Int. Cl. A61K 47/48

(52) U.S. Cl. CPC A61K 47/48646; A61K 47/48715; A61K 47/48384

(57) ABSTRACT

The present invention relates to an antibody-drug conjugate capable of binding to the protein Axl. From one aspect, the invention relates to an antibody-drug conjugate comprising an antibody capable of binding to Axl, said antibody being conjugated to at least one drug which is a pyrrolobenzodiazepme dimer (PBD dimer) drug. The invention also comprises method of treatment and the use of said antibody-drug conjugate for the treatment of cancer.

The present invention relates to an antibody-drug conjugate capable of binding to the protein Axl. From one aspect, the invention relates to an antibody-drug conjugate comprising an antibody capable of binding to Axl, said antibody being conjugated to at least one drug which is a pyrrolobenzodiazepine dimer (PBD dimer) drug. The invention also comprises method of treatment and the use of said antibody-drug conjugate for the treatment of cancer.

### BACKGROUND OF THE INVENTION

“Axl” (also referred to as “Ufo”, “Ark” or “Tyro7”) was cloned from patients with chronic myeloid leukemia as an oncogene triggering the transformation when over-expressed by mouse NIH3T3. It belongs to a family of receptor tyrosine kinases (RTKs) called the TAM (Tyro3, Axl, Mer) family, which includes Tyro3 (Rse, Sky, Dtk, Etk, Brt, Tif), Axl, and Mer (Eyk, Nyk, Tyro-12).

The human protein Axl is a 894 amino acids protein which sequence is represented in the sequence listing as SEQ ID No. 83. Amino acids 1-25 corresponding to the signal peptide, the human protein Axl, without the said peptide signal, is represented in the sequence listing as SEQ ID No. 84.

Gas6, originally isolated as growth arrest-specific gene, is the common ligand for the members of the TAM family. Gas6 exhibits the highest affinity for Axl, followed by Tyro3 and finally by Mer. Gas6 consists in a γ-carboxyglutamate (Gla)-rich domain that mediates binding to phospholipid membranes, four epidermal growth factor-like domains, and two laminin G-like (LG) domains. As many other RTKs, ligand binding results in receptor dimerization and autophosphorylation of tyrosine residues (tyrosine residues 779, 821 and 866 for the receptor Axl) which serve as docking sites for a variety of intracellular signaling molecules. Moreover, the Axl receptor can be activated through a ligand-independent process. This activation can occur when the Axl receptor is overexpressed.

Gas6/Axl signaling has been shown to regulate various cellular processes including cell proliferation, adhesion, migration and survival in a large variety of cells in vitro. In addition, the TAM receptors are involved in the control of innate immunity; they inhibit the inflammatory responses to pathogens in dendritic cells (DCs) and macrophages. They also drive phagocytosis of apoptotic cells by these immune cells and they are required for the maturation and killing activity of natural killer (NK) cells.

Weakly expressed on normal cells, it is predominantly observed in fibroblasts, myeloid progenitor cells, macrophages, neural tissues, cardiac and skeletal muscle where it supports mainly cell survival. The Gas6/Axl system plays an important role in vascular biology by regulating vascular smooth muscle cell homeostasis.

In tumor cells, Axl plays an important role in regulating cellular invasion and migration. Over-expression of Axl is associated not only with poor prognosis but also with increased invasiveness of various human cancers as reported for breast, colon, esophageal carcinoma, hepatocellular, gastric, glioma, lung, melanoma, osteosarcoma, ovarian, prostate, rhabdomyo sarcoma, renal, thyroid and uterine endometrial cancer. In breast cancer, Axl appears to be a strong effector of the Epithelial-to-mesenchymal transition (EMT); EMT program contributes actively to migration and dissemination of cancer cells in the organism.

Axl has also been shown to regulate angiogenesis. Indeed knockdown of Axl in endothelial cells impaired tube formation and migration as well as disturbed specific angiogenic signaling pathways.

More recently several studies on a range of cellular models described the involvement of an Axl overexpression in drug resistance phenomena such as ovarian cancer (Cisplatin), GIST (Imatinib), NSCLC (Doxorubicin, Erlotinib), AML (Doxorubicin/Cisplatin), Breast cancer (Lapatinib), Astrocytoma (Temozolomide, Carboplatin, Vincristine).

In such a context Axl is considered as an interesting target in oncology. Several groups already developed anti-tumoral strategies targeting the gash/Axl axis, either using naked monoclonal antibodies or targeted small molecules.

In this context, the invention relates to an immunoconjugate, also referred as an antibody-drug conjugate (ADC) or conjugate and its use for the treatment of cancer, and more particularly Axl-expressing cancers.

The present invention relates to an ADC comprising a cell binding agent (CBA), preferentially an antibody, conjugated to at least one drug (D), wherein said CBA is capable of binding to Axl.

ADCs combine the binding specificity of a CBA with the potency of drugs such as, for example, cytotoxic agents. The technology associated with the development of monoclonal antibodies, the use of more effective drugs, and the design of chemical linkers to covalently bind these components, has progressed rapidly in recent years.

The use of ADCs allows the local delivery of drugs which, if administered as unconjugated drugs, may result in unacceptable levels of toxicity to normal cells.

In other words, maximal efficacy with minimal toxicity is sought thereby. Efforts to design and refine ADC have focused on the selectivity of CBA as well as drug mechanism of action, drug-linking, drug/CBA ratio (loading), and drug-releasing properties. Drug moieties may impart their cytotoxic and cytostatic effects by mechanisms including tubulin binding, DNA binding, proteasome and/or topoisomerase inhibition. Some cytotoxic drugs tend to be inactive or less active when conjugated to large CBA. ADCs comprising pyrrolobenzodiazepines (PBDs) have been disclosed, for example, in WO 20111/130598.

Each CBA must be characterized separately, an appropriate linker designed, and a suitable cytotoxic agent identified that retains its potency upon delivery to tumor cells. One must consider the antigen density on the cancer target and whether normal tissues express the target antigen. Other considerations include whether the entire ADC is internalized upon binding the target; whether a cytostatic or cytotoxic drug is preferable when considering possible normal tissue exposure and/or the type and stage of the cancer being treated; and, whether the linker connecting the CBA to the drug payload is a cleavable or a non-cleavable linkage. Furthermore, the CBA to drug moiety conjugation ratio must be sufficient without compromising the binding activity of the CBA and/or the potency of the drug.

An ADC is a complex biologic and the challenges to develop an effective ADC remain a significant issue.

### SUMMARY OF THE INVENTION

The present invention intends to address this issue and relates to an ADC comprising cell binding agent (CBA) conjugated to at least one drug (D), wherein said CBA is an antibody capable of binding to Axl and wherein D consists of a pyrrolobenzodiazepine dimer (referred as PBD dimer).

The invention relates to an antibody-drug conjugate having the structural general formula:

CBA-(D)ₙ

wherein:

CBA is an antibody consisting of the 1613F12, or an antigen binding fragment thereof, comprising the three light chain CDRs of sequences SEQ ID No. 1, 2 and 3 and the three heavy chain CDRs of sequences SEQ ID No. 4, 5 and 6; n is 1 to 12; and D is a drug consisting of a pyrrolobenzodiazepine dimer (PBD dimer) having the formulae (AB) or (AC)

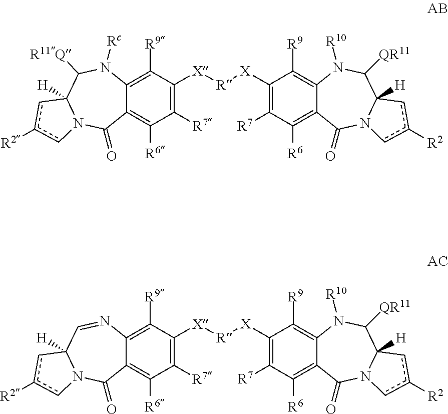

wherein:

the dotted lines indicate the optional presence of a double bond between C1 and C2 or C2 and C3;

R² is independently selected from H, OH, ═O, ═CH₂, CN, R, OR, ═CH—R^{D}, ═C(R^{D})₂, O—SO₂—R, CO₂R and COR, and optionally further selected from halo or dihalo;

where R^{D} is independently selected from R, CO₂R, COR, CHO, CO₂H, and halo;

R⁶ and R⁹ are independently selected from H, R, OH, OR, SH, SR, NH₂, NHR, NRR′, NO₂, Me₃Sn and halo;

R⁷ is independently selected from H, R, OH, OR, SH, SR, NH₂, NHR, NRR′, NO₂, Me₃Sn and halo;

R¹⁰ is a linker connected to CBA;

Q is independently selected from O, S and NH;

R¹¹ is either H, or R or, where Q is O, SO₃M, where M is a metal cation;

R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;

X is O, S or NH;

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted; and

wherein R^{2″}, R^{6″}, R^{7″}, R^{9″}, X″, Q″ and R^{11″} and are as defined according to R², R⁶, R⁷, R⁹, X, Q and R¹¹ respectively, and R^{C} is a capping group.

In one embodiment, 1613F12 is a humanized antibody.

In one embodiment, 1613F12, or an antigen binding fragment thereof, comprises a light chain variable domain of sequence SEQ ID No. 17 or any sequence exhibiting at least 80% identity with SEQ ID No. 17.

In one embodiment, 1613F12, or an antigen binding fragment thereof, comprises a light chain variable domain selected from sequences SEQ ID No. 18 to 28 or any sequence exhibiting at least 80% identity with SEQ ID No. 18 to 28.

In one embodiment, 1613F12, or an antigen binding fragment thereof, comprises a heavy chain variable domain of sequence SEQ ID No. 29 or any sequence exhibiting at least 80% identity with SEQ ID No. 29.

In one embodiment, 1613F12, or an antigen binding fragment thereof, comprises a heavy chain variable domain selected from sequences SEQ ID No. 30 to 49 or any sequence exhibiting at least 80% identity with SEQ ID No. 30 to 49.

In one embodiment, 1613F12, or an antigen binding fragment thereof, comprises a light chain variable domain of sequence SEQ ID No. 81 or any sequence exhibiting at least 80% identity with SEQ ID No. 81, and a heavy chain variable domain of sequence SEQ ID No. 82 or any sequence exhibiting at least 80% identity with SEQ ID No. 82.

In one embodiment, 1613F12 is selected from antibodies, or antigen binding fragments thereof, comprising:

a) a light chain variable domain of sequence SEQ ID No. 19 or any sequence exhibiting at least 80% identity with SEQ ID No. 19, and a heavy chain variable domain of sequence SEQ ID No. 40 or any sequence exhibiting at least 80% identity with SEQ ID No. 40;

b) a light chain variable domain of sequence SEQ ID No. 21 or any sequence exhibiting at least 80% identity with SEQ ID No. 21, and a heavy chain variable domain of sequence SEQ ID No. 40 or any sequence exhibiting at least 80% identity with SEQ ID No. 40;

c) a light chain variable domain of sequence SEQ ID No. 27 or any sequence exhibiting at least 80% identity with SEQ ID No. 27, and a heavy chain variable domain of sequence SEQ ID No. 32 or any sequence exhibiting at least 80% identity with SEQ ID No. 32; or

d) a light chain variable domain of sequence SEQ ID No. 28 or any sequence exhibiting at least 80% identity with SEQ ID No. 28, and a heavy chain variable domain of sequence SEQ ID No. 32 or any sequence exhibiting at least 80% identity with SEQ ID No. 32.

In another embodiment, R¹⁰ is:

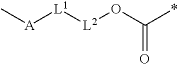

wherein A is a connecting group connecting L¹ to CBA, L¹ is a cleavable linker, L² is a covalent bond or together with —OC(═O)— forms a self-immolative linker, and the asterisk indicates the point of attachment to the N10 position of D.

In an embodiment, A is selected from:

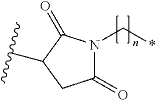

wherein the asterisk indicates the point of attachment to L¹, the wavy line indicates the point of attachment to CBA, and n is 0 to 6;

or

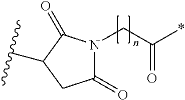

wherein the asterisk indicates the point of attachment to L¹, the wavy line indicates the point of attachment to CBA, and n is 0 to 6;

or

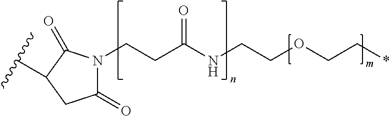

wherein the asterisk indicates the point of attachment to L¹, the wavy line indicates the point of attachment to CBA, n is 0 or 1, and m is 0 to 30;

or

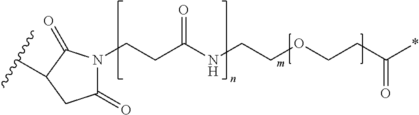

wherein the asterisk indicates the point of attachment to L¹, the wavy line indicates the point of attachment to CBA, n is 0 or 1, and m is 0 to 30.

In an embodiment, the CBA is connected to A through a thioether bond formed from a cysteine thiol residue of CBA and a malemide group of A.

In an embodiment, L₁ comprises a dipeptide —NH—X₁—X₂—CO— wherein the group —X₁—X₂— is selected from -Phe-Lys-, -Val-Ala-, -Val-Lys-, -Ala-Lys-, -Val-Cit-, -Phe-Cit-, -Leu-Cit-, -Ile-Cit-, -Phe-Arg-, -Trp-Cit-, wherein Cit is citrulline.

In an embodiment, —C(═O)O— and L₂ together form the group:

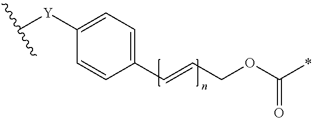

wherein the asterisk indicates the point of attachment to the N10 position of D, the wavy line indicates the point of attachment to the linker L¹, Y is —N(H)—, —O—, —C(═O)N(H)— or —C(═O)O—, and n is 0 to 3.

In an embodiment, L₁ and L₂ together with —C(═O)O— comprise a group selected from:

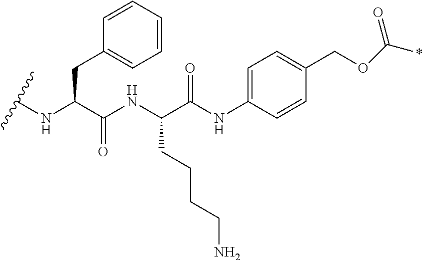 [SMILES: *N[C@@H](Cc1ccccc1)C(=O)N[C@@H](CCCCN)C(=O)Nc1ccc(COC(*)=O)cc1]

wherein the asterisk indicates the point of attachment to the N10 position of D, and the wavy line indicates the point of attachment to the remaining portion of the linker L¹ or the point of attachment to A;

or

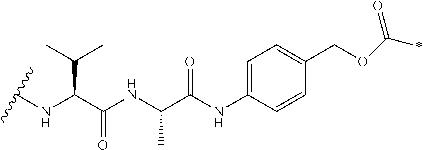 [SMILES: *N[C@H](C(=O)N[C@@H](C)C(=O)Nc1ccc(COC(*)=O)cc1)C(C)C]

wherein the asterisk and the wavy line are as defined above;

or

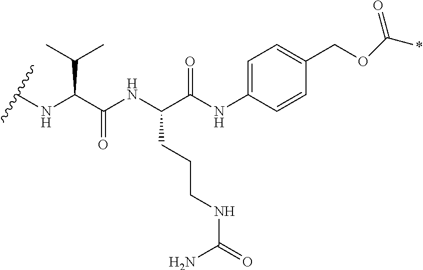 [SMILES: *N[C@H](C(=O)N[C@@H](CCCNC(N)=O)C(=O)Nc1ccc(COC(*)=O)cc1)C(C)C]

wherein the asterisk and the wavy line are as defined above.

In an embodiment, D is selected from:

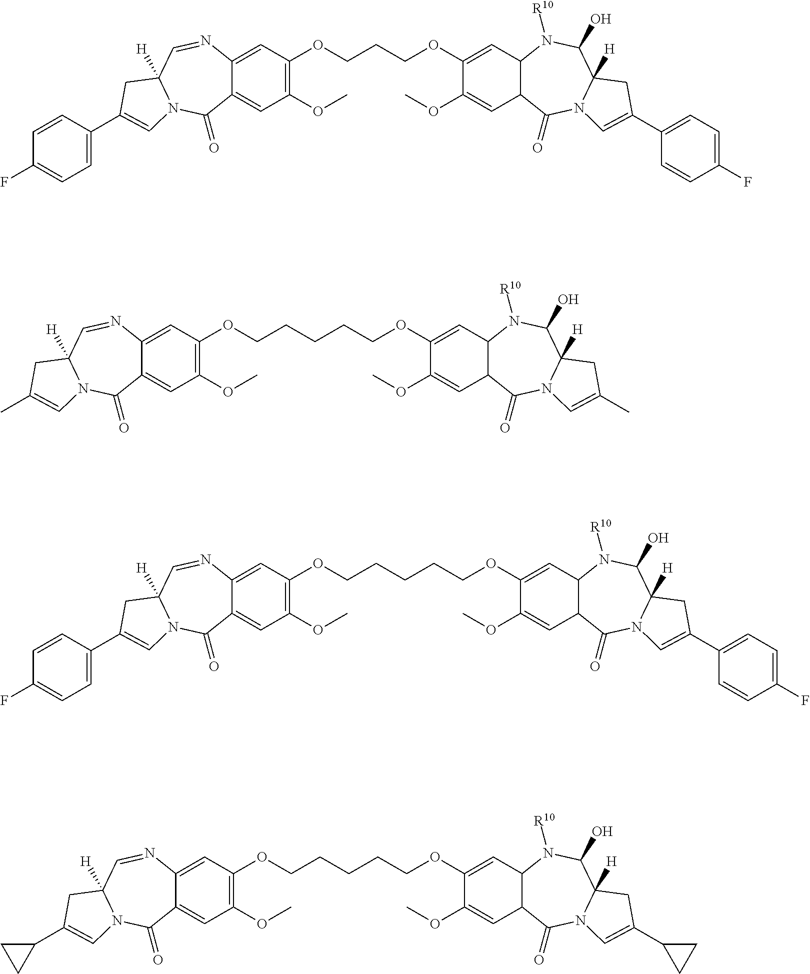

In a preferred embodiment, the ADC is of the structural general formula selected from:

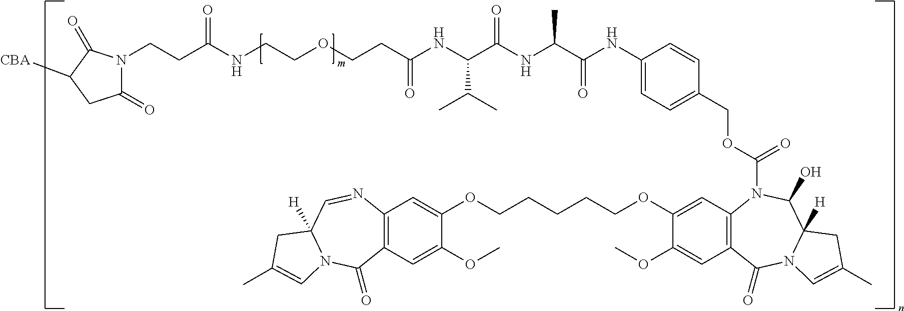

wherein CBA consists of the 1613F12, or an antigen binding fragment thereof, m is 0 to 30, and n is 1 to 12;

or

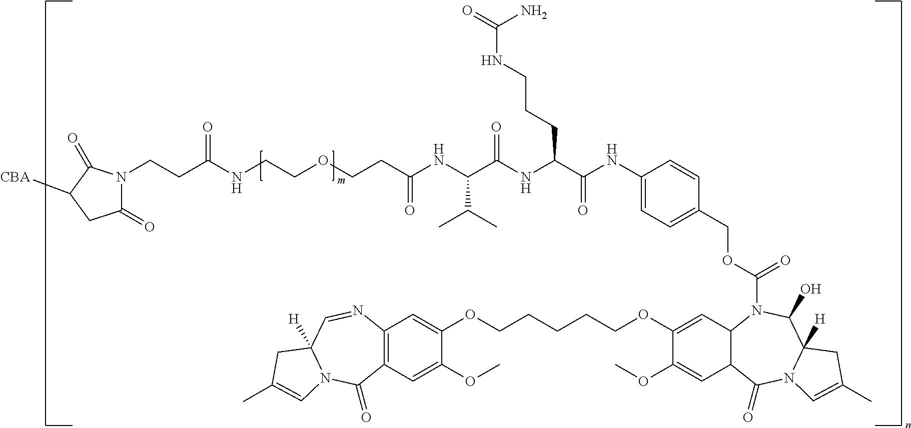

wherein CBA consists of the 1613F12, or an antigen binding fragment thereof, m is 0 to 30, and n is 1 to 12.

In another preferred embodiment, the ADC is of the structural general formula selected from:

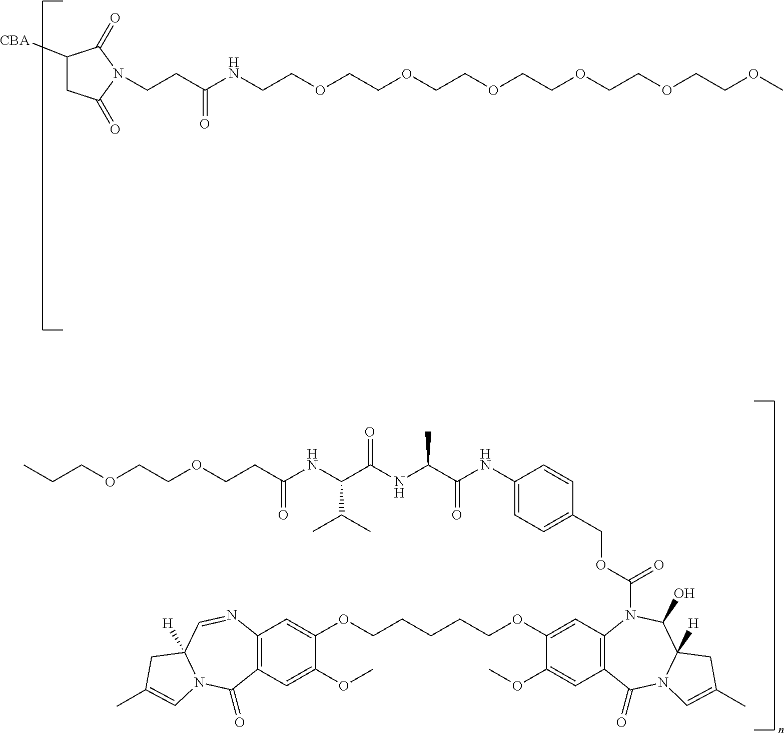

wherein CBA consists of the 1613F12, or an antigen binding fragment thereof, and n is 1 to 12;

or

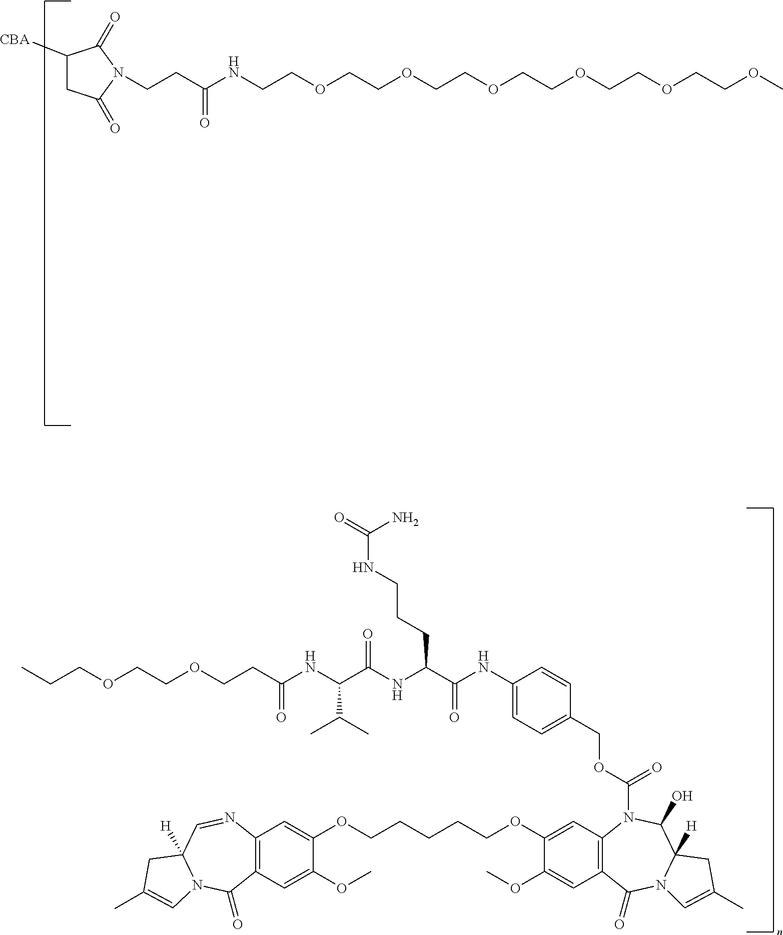

wherein CBA consists of the 1613F12, or an antigen binding fragment thereof, and n is 1 to 12.

In an embodiment, n is 2.

In an embodiment, n is 4.

The invention also relates to such an ADC for use in the treatment of an Axl-expressing cancer.

The invention also relates to a composition comprising at least an ADC according to the invention.

In an embodiment, such a composition is a pharmaceutical composition further comprising a pharmaceutically acceptable vehicle.

The invention also relates to such a composition for use in the treatment of an Axl-expressing cancer.

The invention relates to the use of an ADC or of a composition for the treatment of an Axl-expressing cancer.

In an embodiment, said Axl-expressing cancer is a cancer chosen from breast, colon, esophageal carcinoma, hepatocellular, gastric, glioma, lung, melanoma, osteosarcoma, ovarian, prostate, rhabdomyosarcoma, renal, thyroid, uterine endometrial cancer, mesothelioma, oral squamous carcinoma and any drug resistant cancer.

The invention also relates to a method for the treatment of an Axl-expressing cancer in a subject, comprising administering to the subject an effective amount of at least the ADC or the composition as described.

The invention also relates to a kit comprising at least i) an ADC and/or a composition as described and ii) a syringe or vial or ampoule in which the said ADC and/or composition is disposed.

### DETAILED DESCRIPTION OF THE INVENTION

I—The Cell Binding Agent (CBA)

According to the invention, the CBA consists of a monoclonal antibody, or an antigen binding fragment thereof, capable of binding to Axl and thereafter named 1613F12 or Axl antibody.

The 1613F12 is derived from the hybridoma of murine origin filed with the French collection for microorganism cultures (CNCM, Pasteur Institute, Paris, France) on Jul. 28, 2011, under number 1-4505. Said hybridoma was obtained by the fusion of Balb/C immunized mice splenocytes/lymphocytes and cells of the myeloma Sp 2/O—Ag 14 cell line.

In an embodiment, the Axl antibody of the invention consists preferentially of a murine antibody, then referred as m1613F12.

In an embodiment, the Axl antibody of the invention consists preferentially of a chimeric antibody, then referred as c1613F12.

In an embodiment, the Axl antibody of the invention consists preferentially of a humanized antibody, then referred as hz1613F12.

For the avoidance of doubt, in the following specification, the expressions “Axl antibody” and “1613F12” are similar and include (without contrary specification) the murine, the chimeric and the humanized versions of 1613F12. When necessary, the prefix m-(murine), c-(chimeric) or hz-(humanized) is used.

The Axl antibody, or an antigen binding fragment thereof, is capable of binding to the human protein Axl. More particularly, the said target is an epitope located into the extracellular domain of Axl (referred as the Axl ECD domain).

The ECD of the human protein Axl is a 451 amino acids fragment, corresponding to amino acids 1-451 of the sequence SEQ ID No. 83, which sequence is represented in the sequence listing as SEQ ID No. 85. Amino acids 1-25 corresponding to the signal peptide, the ECD of the human protein Axl without the signal peptide corresponds to the amino acids 26-451 of the sequence SEQ ID No.83, represented by the sequence SEQ ID No. 86.

In another embodiment, of the invention, the said Axl antibody is internalized following its binding to said human protein Axl.

By “antigen binding fragment” of an antibody according to the invention, it is intended to indicate any peptide, polypeptide, or protein retaining the ability to bind to the target (also generally referred as antigen) of the antibody, and more preferably comprising the amino acid sequences of the 6 CDRs of said antibody.

In a preferred embodiment, such “antigen binding fragments” are selected in the group consisting of Fv, scFv (sc for single chain), Fab, F(ab′)₂, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab)₂-PEG or Fab′-PEG) (“PEG” for Poly(Ethylene) Glycol), or by incorporation in a liposome, said fragments having at least one of the characteristic CDRs of the antibody according to the invention. Preferably, said “antigen binding fragments” will be constituted or will comprise a partial sequence of the heavy or light variable chain of the antibody from which they are derived, said partial sequence being sufficient to retain the same specificity of binding as the antibody from which it is descended and a sufficient affinity, preferably at least equal to 1/100, in a more preferred manner to at least 1/10, of the affinity of the antibody from which it is descended, with respect to the target. Such a functional fragment will contain at the minimum 5 amino acids, preferably 10, 15, 25, 50 and 100 consecutive amino acids of the sequence of the antibody from which it is descended. In an embodiment of the invention, said antigen binding fragment comprises the amino acid sequences corresponding to the three light chain CDRs of sequences SEQ ID No. 1, 2 and 3 and to the three heavy chain CDRs of sequences SEQ ID No. 4, 5 and 6.

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

In the present application, the epitope is localized into the extracellular domain of the human protein Axl.

According to a preferred embodiment of the invention, the antibody, or an antigen binding fragment thereof, binds to an epitope localized into the human protein Axl extracellular domain, preferably having the sequence SEQ ID NO. 85 or 86 or natural variant sequence thereof.

Generally speaking, an antibody which “binds”, or the like, means an antibody capable of binding to the antigen with sufficient affinity such that the antibody is useful in targeting a cell expressing the antigen. The binding of the Axl antibody can be determined, without limitation, by fluorescence activated cell sorting (FACS), ELISA, radioimmunoprecipitation (RIA) or BIACORE or any other methods known by the person skilled in the art. More particularly, by “binding”, “binds”, or the like, it is intended that the antibody, or antigen-binding fragment thereof, forms a complex with an antigen that is relatively stable under physiologic conditions. Specific binding can be characterized by an equilibrium dissociation constant of at least about 1·10⁻⁶ M or less. Methods for determining whether two molecules specifically bind are well known in the art and include, for example, equilibrium dialysis, surface plasmon resonance, and the like. For the avoidance of doubt, it does not mean that the said antibody could not bind or interfere, at a low level, to another antigen. Nevertheless, as a preferred embodiment, the said antibody binds only to the said antigen.

The Axl antibody also presents a high ability to be internalized following Axl binding. Such antibody is interesting as one of the ADC components, so it addresses the linked cytotoxic into the targeted cancer cells. Once internalized the cytotoxic triggers cancer cell death.

Important keys to success with ADC therapy are thought to be the target antigen specificity and the internalization of the antibody complexes into the cancer cells.

Obviously non-internalizing antigens are less effective than internalizing antigens to delivers cytotoxic agents. Internalization processes are variable across antigens and depend on multiple parameters that can be influenced by binding proteins. Cell-surface RTKs constitute an interesting antigens family to investigate for such an approach.

In the biomolecule, the cytotoxic brings the cytotoxic activity and the used antigen binding protein brings its specificity against cancer cells, as well as a vector for entering within the cells to correctly address the cytotoxic.

Thus to improve the ADC molecule, the antibody must exhibit high ability to internalize into the targeted cancer cells. The efficiency with which the antibodies mediated internalisation differs significantly depending on the epitope targeted.

Antibodies in the sense of the invention also include certain antibody fragments, thereof. The said antibody fragments exhibit the desired binding specificity and affinity, regardless of the source or immunoglobulin type (i.e., IgG, IgE, IgM, IgA, etc.), i.e., they are capable of binding specifically the Axl protein with an affinity comparable to the full-length antibodies of the invention.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Kohler and Milstein (Nature, 256:495-497, 1975).

The term “monoclonal antibody” or “Mab” as used herein refers to an antibody molecule that is directed against a specific antigen and which may be produced by a single clone of B cells or hybridoma. Monoclonal antibodies may also be recombinant, i.e. produced by protein engineering. In addition, in contrast with preparations of polyclonal antibodies which typically include various antibodies directed against various determinants, or epitopes, each monoclonal antibody is directed against a single epitope of the antigen. The invention relates to antibodies isolated or obtained by purification from natural sources or obtained by genetic recombination or chemical synthesis.

The Axl antibody of the invention, or an antigen binding fragment thereof, comprises the three light chain CDRs comprising the sequences SEQ ID Nos. 1, 2 and 3, or any sequence exhibiting at least 90%, preferably 95% and 98% identity with SEQ ID Nos. 1, 2 and 3; and the three heavy chain CDRs comprising the sequences SEQ ID Nos. 4, 5 and 6, or any sequence exhibiting at least 90%, preferably 95% and 98% identity with SEQ ID Nos. 4, 5 and 6.

In an embodiment of the invention, the Axl antibody, or an antigen binding fragment thereof, comprises the three light chain CDRs comprising respectively the sequences SEQ ID Nos. 1, 2 and 3; and the three heavy chain CDRs comprising respectively the sequences SEQ ID Nos. 4, 5 and 6.

In a preferred aspect, by CDR regions or CDR(s), it is intended to indicate the hypervariable regions of the heavy and light chains of the immunoglobulins as defined by IMGT. Without any contradictory mention, the CDRs will be defined in the present specification according to the IMGT numbering system.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cystein 23 (1st-CYS), tryptophan 41 (CONSERVED-TRP), hydrophobic amino acid 89, cystein 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3Dstructure-DB [Kaas, Q., Ruiz, M. and Lefranc, M.-P., T cell receptor and MHC structural data. Nucl. Acids. Res., 32, D208-D210 (2004)].

It must be understood that, without contradictory specification in the present specification, complementarity-determining regions or CDRs, mean the hypervariable regions of the heavy and light chains of immunoglobulins as defined according to the IMGT numbering system.

In the sense of the present invention, the “percentage identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981) [Ad. App. Math. 2:482], by means of the local homology algorithm of Neddleman and Wunsch (1970) [J. Mol. Biol. 48:443], by means of the similarity search method of Pearson and Lipman (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P).

The percentage identity between two nucleic acid or amino acid sequences is determined by comparing the two optimally-aligned sequences in which the nucleic acid or amino acid sequence to compare can have additions or deletions compared to the reference sequence for optimal alignment between the two sequences. Percentage identity is calculated by determining the number of positions at which the amino acid nucleotide or residue is identical between the two sequences, preferably between the two complete sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/b12.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

For the amino acid sequence exhibiting at least 90%, preferably 95% and 98% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below.

Equivalent amino acids can be determined either on their structural homology with the amino acids for which they are substituted or on the results of comparative tests of biological activity between the various antigen binding proteins likely to be generated.

As a non-limiting example, table 1 below summarizes the possible substitutions likely to be carried out without resulting in a significant modification of the biological activity of the corresponding modified antigen binding protein; inverse substitutions are naturally possible under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

In an embodiment of the invention, the Axl antibody consists of the m1613F12, or an antigen binding fragment thereof, comprising i) a light chain variable domain of sequence SEQ ID No. 7, or any sequence exhibiting at least 80%, preferably 85%, 90%, 95% and 98% identity with SEQ ID No. 7; and/or ii) a heavy chain variable domain of sequence SEQ ID No. 8, or any sequence exhibiting at least 80%, preferably 85%, 90%, 95% and 98% identity with SEQ ID No. 8.

By “any sequence exhibiting at least 80%, preferably 85%, 90%, 95% and 98% identity with the sequence of a light (or heavy, respectively) chain variable domain, it is intended to designate the sequences exhibiting the three light (or heavy, respectively) chain CDRs and, in addition, exhibiting at least 80%, preferably 85%, 90%, 95% and 98%, identity with the full sequence of the light (or heavy, respectively) chain outside the sequences corresponding to the CDRs.

For more clarity, table 2a below summarizes the various amino acid sequences corresponding to the Axl antibody of the invention (with m.=murine).

TABLE 2a

| | CDR | | | |
|---|---|---|---|---|
| | numbering | Heavy chain | Light chain | SEQ ID NO. |
| 1613F12 | IMGT | | CDR-L1 | 1 |
| | | | CDR-L2 | 2 |
| | | | CDR-L3 | 3 |
| | | CDR-H1 | | 4 |
| | | CDR-H2 | | 5 |
| | | CDR-H3 | | 6 |
| | | | m. variable domain | 7 |
| | | m. variable | | 8 |
| | | domain | | |

In an embodiment of the invention, the Axl antibody consists of the c1613F12, or an antigen binding fragment thereof, comprising i) a light chain variable domain of sequence SEQ ID No. 7, or any sequence exhibiting at least 80%, preferably 85%, 90%, 95% and 98% identity with SEQ ID No. 7; and/or ii) a heavy chain variable domain of sequence SEQ ID No. 8, or any sequence exhibiting at least 80%, preferably 85%, 90%, 95% and 98% identity with SEQ ID No. 8.

A chimeric antibody is one containing a natural variable region (light chain and heavy chain) derived from an antibody of a given species in combination with constant regions of the light chain and the heavy chain of an antibody of a species heterologous to said given species.

The antibodies, or chimeric fragments of same, can be prepared by using the techniques of recombinant genetics. For example, the chimeric antibody could be produced by cloning recombinant DNA containing a promoter and a sequence coding for the variable region of a nonhuman monoclonal antibody of the invention, notably murine, and a sequence coding for the human antibody constant region. A chimeric antibody according to the invention coded by one such recombinant gene could be, for example, a mouse-human chimera, the specificity of this antibody being determined by the variable region derived from the murine DNA and its isotype determined by the constant region derived from human DNA. Refer to Verhoeyn et al. (BioEssays, 8:74, 1988) for methods for preparing chimeric antibodies.

In an embodiment of the invention, the Axl antibody consists of the hz1613F12, or an antigen binding fragment of same, comprising the three light chain CDRs comprising the sequences SEQ ID No. 1, 2 and 3, or any sequence exhibiting at least 80%, preferably 85%, 90%, 95% and 98% identity with SEQ ID No. 1, 2 and 3; and the three heavy chain CDRs comprising the sequences SEQ ID No. 4, 5 and 6, or any sequence exhibiting at least 80%, preferably 85%, 90%, 95% and 98% identity with SEQ ID No. 4, 5 and 6.

In an embodiment of the invention, hz1613F12, or an antigen binding fragment thereof, comprises the three light chain CDRs comprising respectively the sequences SEQ ID Nos. 1, 2 and 3; and the three heavy chain CDRs comprising respectively the sequences SEQ ID Nos. 4, 5 and 6.

“Humanized antibodies” means an antibody that contains CDR regions derived from an antibody of nonhuman origin, the other parts of the antibody molecule being derived from one (or several) human antibodies. In addition, some of the skeleton segment residues (called FR) can be modified to preserve binding affinity (Jones et al., Nature, 321:522-525, 1986; Verhoeyen et al., Science, 239:1534-1536, 1988; Riechmann et al., Nature, 332:323-327, 1988).

The humanized antibodies of the invention or fragments of same can be prepared by techniques known to a person skilled in the art (such as, for example, those described in the documents Singer et al., J. Immun., 150:2844-2857, 1992; Mountain et al., Biotechnol. Genet. Eng. Rev., 10:1-142, 1992; and Bebbington et al., Bio/Technology, 10:169-175, 1992). Such humanized antibodies are preferred for their use in methods involving in vitro diagnoses or preventive and/or therapeutic treatment in vivo. Other humanization techniques, also known to a person skilled in the art, such as, for example, the “CDR grafting” technique described by PDL in patents EP 0 451 261, EP 0 682 040, EP 0 939 127, EP 0 566 647 or U.S. Pat. No. 5,530,101, U.S. Pat. No. 6,180,370, U.S. Pat. No. 5,585,089 and U.S. Pat. No. 5,693,761. U.S. Pat. Nos. 5,639,641 or 6,054,297, 5,886,152 and 5,877,293 can also be cited.

In an embodiment of the invention, hz1613F12, or an antigen binding fragment, comprises a light chain variable domain consisting of the sequence SEQ ID No. 17, or any sequence exhibiting at least 80%, preferably 85%, 90%, 95% and 98% identity with SEQ ID No. 17; and the three heavy chain CDRs consisting of sequences SEQ ID No. 4, 5 and 6.

In another embodiment of the invention, hz1613F12, or an antigen binding fragment thereof, comprises a light chain variable domain of sequence selected in the group consisting of SEQ ID No. 18 to 28, or any sequence exhibiting at least 80%, preferably 85%, 90%, 95% and 98% identity with SEQ ID No. 18 to 28; and the three heavy chain CDRs consisting of SEQ ID No. 4, 5 and 6.

In another embodiment of the invention, hz1613F12, or an antigen binding fragment thereof, comprises a light chain variable domain of sequence SEQ ID No. 81, or any sequence exhibiting at least 80%, preferably 85%, 90%, 95% and 98% identity with SEQ ID No. 81; and the three heavy chain CDRs consisting of SEQ ID No. 4, 5 and 6.

In order to illustrate the identity percentage as defined before, by “any sequence exhibiting at least 80%, preferably 85%, 90%, 95% and 98% identity with SEQ ID No. 17, 18 to 28 or 81”, its is intended to designate the sequences exhibiting the three light chain CDRs SEQ ID No. 1, 2 and 3 and, in addition, exhibiting at least 80%, preferably 85%, 90%, 95% and 98%, identity with the full sequence SEQ ID No. 17, 18 to 28 or 81 outside the sequences corresponding to the CDRs (i.e. SEQ ID No. 1, 2 and 3).

For more clarity, table 2b below summarizes the various amino acid sequences corresponding to the humanized Axl antibody light chain (VL) of the invention (with hz.=humanized)

TABLE 2b

| | Version | SEQ ID NO. |
|---|---|---|
| hz1613F12 VL | consensus | 17 |
| | VL1 | 18 |
| | VL1 I2V | 19 |
| | VL1 M4I | 20 |
| | VL2.1 | 21 |
| | VL2.1 V49T | 22 |
| | VL2.1 P50N | 23 |
| | VL2.2 | 24 |
| | VL2.2 V49T | 25 |
| | VL2.2 P50N | 26 |
| | VL2.3 | 27 |
| | VL3 | 28 |
| | Consensus 2 | 81 |

In an embodiment of the invention, the CBA consists of an antibody, or an antigen binding fragment thereof, comprising a light chain variable domain selected in the group consisting of:

i) a light chain variable domain of sequence SEQ ID No. 17 or any sequence exhibiting at least 80% identity with SEQ ID No.7,

ii) a light chain variable domain of sequence SEQ ID No. 81 or any sequence exhibiting at least 80% identity with SEQ ID No. 81; and

iii) a light chain variable domain of sequence SEQ ID No. 18 to 28 or any sequence exhibiting at least 80% identity with SEQ ID No. 18 to 28.

In an embodiment of the invention, hz1613F12, or an antigen binding fragment, comprises a heavy chain variable domain consisting of the sequence SEQ ID No. 29, or any sequence exhibiting at least 80%, preferably 85%, 90%, 95% and 98% identity with SEQ ID No. 29; and the three light chain CDRs consisting of sequences SEQ ID No. 1, 2 and 3.

In another embodiment of the invention, hz1613F12, or an antigen binding fragment thereof, comprises a heavy chain variable domain of sequence selected in the group consisting of SEQ ID No. 30 to 49, or any sequence exhibiting at least 80%, preferably 85%, 90%, 95% and 98% identity with SEQ ID No. 30 to 49; and the three light chain CDRs consisting of SEQ ID No. 1, 2 and 3.

In another embodiment of the invention, hz1613F12, or an antigen binding fragment thereof, comprises a heavy chain variable domain of sequence SEQ ID No. 82, or any sequence exhibiting at least 80%, preferably 85%, 90%, 95% and 98% identity with SEQ ID No. 82; and the three light chain CDRs consisting of SEQ ID No. 1, 2 and 3.

In order to illustrate the identity percentage as defined before, by “any sequence exhibiting at least 80%, preferably 85%, 90%, 95% and 98% identity with SEQ ID No. 29, 30 to 49 or 82”, its is intended to designate the sequences exhibiting the three light chain CDRs SEQ ID No. 1, 2 and 3 and, in addition, exhibiting at least 80%, preferably 85%, 90%, 95% and 98%, identity with the full sequence SEQ ID No. 29, 30 to 49 or 82 outside the sequences corresponding to the CDRs (i.e. SEQ ID No. 2, 3 and 4).

For more clarity, table 2c below summarizes the various amino acid sequences corresponding to the humanized antigen binding protein heavy chain (VH) of the invention (with hz.=humanized)

TABLE 2c

| | Version | SEQ ID NO. |
|---|---|---|
| hz1613F12 VH | consensus | 29 |
| | VH1 | 30 |
| | VH1 M39I | 31 |
| | VH1 W55R N66K | 32 |
| | VH1 I84S | 33 |
| | VH1 S85N | 34 |
| | VH1 I84N S85N | 35 |
| | VH2.1 | 36 |
| | VH2.1 Q3H | 37 |
| | VH2.1 W55R | 38 |
| | VH2.1 N66K | 39 |
| | VH2.1 W55R N66K | 40 |
| | VH2.1 R80S | 41 |
| | VH2.1 N66K R80S | 42 |
| | VH2.2 | 43 |
| | VH2.2 M89L | 44 |
| | VH2.3 | 45 |
| | VH2.3 W55R | 46 |
| | VH2.3 Q3H W55R | 47 |
| | VH2.4 | 48 |
| | VH3 | 49 |
| | Consensus 2 | 82 |

In an embodiment of the invention, the CBA consists of an antibody, or an antigen binding fragment thereof, comprising a light chain variable domain selected in the group consisting of:

i) a heavy chain variable domain of sequence SEQ ID No. 29 or any sequence exhibiting at least 80% identity with SEQ ID No.29,

ii) a heavy chain variable domain of sequence SEQ ID No. 82 or any sequence exhibiting at least 80% identity with SEQ ID No. 82; and

iii) a heavy chain variable domain of sequence SEQ ID No. 30 to 49 or any sequence exhibiting at least 80% identity with SEQ ID No. 30 to 49.

In an embodiment of the invention, hz1613F12, or an antigen binding fragment thereof, comprises a light chain variable domain of sequence selected in the group consisting of SEQ ID No. 17 to 28 and 81, or any sequence exhibiting at least 80%, preferably 85%, 90%, 95% and 98% identity with SEQ ID No. 17 to 28 and 81; and a heavy chain variable domain of sequence selected in the group consisting of SEQ ID No. 29 to 49 and 82, or any sequence exhibiting at least 80%, preferably 85%, 90%, 95% and 98% identity with SEQ ID No. 29 to 49 and 82.

In an embodiment of the invention, the CBA consists of an antibody, or an antigen binding fragment thereof, comprising:

i) a light chain variable domain of sequence selected from SEQ ID No. 17 to 28 or 81 or any sequence exhibiting at least 80% identity with SEQ ID No.17 to 28 or 81; and

ii) a heavy chain variable domain of sequence selected from SEQ ID No. 29 to 49 and 82 or any sequence exhibiting at least 80% identity with SEQ ID No.29 to 49 and 82.

Table 3a below summarizes the various nucleotide sequences concerning the CBA of the invention (with m=Murine).

TABLE 3a

| | CDR | | | |
|---|---|---|---|---|
| Antibody | numbering | Heavy chain | Light chain | SEQ ID NO. |
| 1613F12 | IMGT | | CDR-L1 | 9 |
| | | | CDR-L2 | 10 |
| | | | CDR-L3 | 11 |
| | | CDR-H1 | | 12 |
| | | CDR-H2 | | 13 |
| | | CDR-H3 | | 14 |
| | | | m variable domain | 15 |
| | | m variable | | 16 |
| | | domain | | |

For more clarity, table 3b below summarizes the various nucleotide sequences corresponding to hz1613F12 light chain (VL) of the invention.

TABLE 3b

| | Version | SEQ ID NO. |
|---|---|---|
| hz1613F12 VL | VL1 | 50 |
| | VL1 I2V | 51 |
| | VL1 M4I | 52 |
| | VL2.1 | 53 |
| | VL2.1 V49T | 54 |
| | VL2.1 P50N | 55 |
| | VL2.2 | 56 |
| | VL2.2 V49T | 57 |
| | VL2.2 P50N | 58 |
| | VL2.3 | 59 |
| | VL3 | 60 |

For more clarity, table 3c below summarizes the various nucleotide sequences corresponding to hz1613F12 heavy chain (VH) of the invention.

TABLE 3c

| | Version | SEQ ID NO. |
|---|---|---|
| hz1613F12 VH | VH1 | 61 |
| | VH1 M39I | 62 |
| | VH1 W55R N66K | 63 |
| | VH1 I84S | 64 |
| | VH1 S85N | 65 |
| | VH1 I84N S85N | 66 |
| | VH2.1 | 67 |
| | VH2.1 Q3H | 68 |
| | VH2.1 W55R | 69 |
| | VH2.1 N66K | 70 |
| | VH2.1 W55R N66K | 71 |
| | VH2.1 | 72 |
| | VH2.1 N66K R80S | 73 |
| | VH2.2 | 74 |
| | VH2.2 M89L | 75 |
| | VH2.3 | 76 |
| | VH2.3 W55R | 77 |
| | VH2.3 Q3H W55R | 78 |
| | VH2.4 | 79 |
| | VH3 | 80 |

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, defining a fragment or a region of a nucleic acid, containing unnatural nucleotides or not, and being either a double-strand DNA, a single-strand DNA or transcription products of said DNAs.

The sequences of the present invention have been isolated and/or purified, i.e., they were sampled directly or indirectly, for example by a copy, their environment having been at least partially modified. Isolated nucleic acids obtained by recombinant genetics, by means, for example, of host cells, or obtained by chemical synthesis should also be mentioned here.

II—The Drug (D)

Suitable drug moieties may be those PBD dimers described in WO 2011/130598. Thus, preferred drug moieties (D) of the present invention are those having the formulae (AB) or (AC):

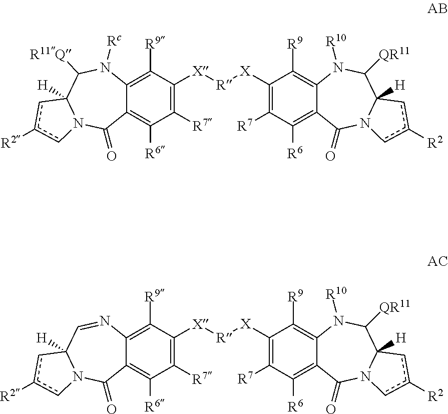

wherein:

the dotted lines indicate the optional presence of a double bond between C1 and C2 or C2 and C3;

R² is independently selected from H, OH, ═O, ═CH₂, CN, R, OR, ═CH—R^{D}, ═C(R^{D})₂, O—SO₂—R, CO₂R and COR, and optionally further selected from halo or dihalo;

where R^{D} is independently selected from R, CO₂R, COR, CHO, CO₂H, and halo;

R⁶ and R⁹ are independently selected from H, R, OH, OR, SH, SR, NH₂, NHR, NRR′, NO₂, Me₃Sn and halo;

R⁷ is independently selected from H, R, OH, OR, SH, SR, NH₂, NHR, NRR′, NO₂, Me₃Sn and halo;

R¹⁰ is a linker connected to a modulator or fragment or derivative thereof, as described above;

Q is independently selected from O, S and NH;

R¹¹ is either H, or R or, where Q is O, SO₃M, where M is a metal cation;

R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted; and

wherein R^{2″}, R^{6″}, R^{7″}, R^{9″}, X″, Q″ and R^{11″} and are as defined according to R², R⁶, R⁷, R⁹, X, Q and R¹¹ respectively, and R^{C} is a capping group.

Double Bond

In one embodiment, there is no double bond present between C1 and C2, and C2 and C3.

In one embodiment, the dotted lines indicate the optional presence of a double bond between C2 and C3, as shown below:

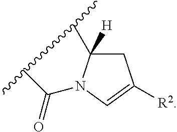

In one embodiment, a double bond is present between C2 and C3 when R² is C₅₋₂₀ aryl or C₁₋₁₂ alkyl.

In one embodiment, the dotted lines indicate the optional presence of a double bond between C1 and C2, as shown below:

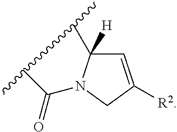

In one embodiment, a double bond is present between C1 and C2 when R² is C₅₋₂₀ aryl or C₁₋₁₂ alkyl.

R²

In one embodiment, R² is independently selected from H, OH, ═O, ═CH₂, CN, R, OR, ═CH—R^{D}, ═C(R^{D})₂, O—SO₂—R, CO₂R and COR, and optionally further selected from halo or dihalo.

In one embodiment, R² is independently selected from H, OH, ═O, ═CH₂, CN, R, OR, ═CH—R^{D}, ═C(R^{D})₂, O—SO₂—R, CO₂R and COR.

In one embodiment, R² is independently selected from H, ═O, ═CH₂, R, ═CH—R^{D}, and ═C(R^{D})₂.

In one embodiment, R² is independently H.

In one embodiment, R² is independently=O.

In one embodiment, R² is independently=CH₂.

In one embodiment, R² is independently=CH—R^{D}. Within the PBD compound, the group ═CH—R¹ may have either configuration shown below:

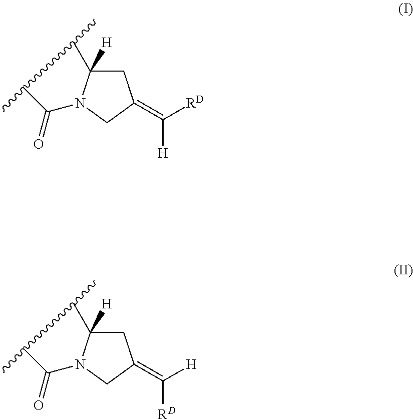

In one embodiment, the configuration is configuration (I).

In one embodiment, R² is independently═C(R^{D})₂.

In one embodiment, R² is independently=CF₂.

In one embodiment, R² is independently R.

In one embodiment, R² is independently optionally substituted C₅₋₂₀ aryl.

In one embodiment, R² is independently optionally substituted C₁₋₁₂ alkyl.

In one embodiment, R² is independently optionally substituted C₅₋₂₀ aryl.

In one embodiment, R² is independently optionally substituted C₅₋₇ aryl.

In one embodiment, R² is independently optionally substituted C₈₋₁₀ aryl.

In one embodiment, R² is independently optionally substituted phenyl.

In one embodiment, R² is independently optionally substituted napthyl.

In one embodiment, R² is independently optionally substituted pyridyl.

In one embodiment, R² is independently optionally substituted quinolinyl or isoquinolinyl.

In one embodiment, R² bears one to three substituent groups, with 1 and 2 being more preferred, and singly substituted groups being most preferred. The substituents may be any position.

Where R² is a C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

In one embodiment, R² is selected from:

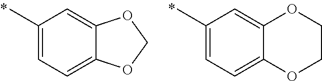

wherein the asterisk indicates the point of attachment.

Where R² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

In one embodiment, where R² is optionally substituted, the substituents are selected from those substituents given in the substituent section below.

Where R is optionally substituted, the substituents are preferably selected from: Halo, Hydroxyl, Ether, Formyl, Acyl, Carboxy, Ester, Acyloxy, Amino, Amido, Acylamido, Aminocarbonyloxy, Ureido, Nitro, Cyano and Thioether.

In one embodiment, where R or R² is optionally substituted, the substituents are selected from the group consisting of R, OR, SR, NRR′, NO₂, halo, CO₂R, COR, CONH₂, CONHR, and CONRR′.

Where R² is C₁₋₁₂ alkyl, the optional substituent may additionally include C₃₋₂₀ heterocyclyl and C₅₋₂₀ aryl groups.

Where R² is C₃₋₂₀ heterocyclyl, the optional substituent may additionally include C₁₋₁₂ alkyl and C₅₋₂₀ aryl groups.

Where R² is C₅₋₂₀ aryl groups, the optional substituent may additionally include C₃₋₂₀ heterocyclyl and C₁₋₁₂ alkyl groups.

It is understood that the term “alkyl” encompasses the sub-classes alkenyl and alkynyl as well as cycloalkyl. Thus, where R² is optionally substituted C₁₋₁₂ alkyl, it is understood that the alkyl group optionally contains one or more carbon-carbon double or triple bonds, which may form part of a conjugated system. In one embodiment, the optionally substituted C₁₋₁₂ alkyl group contains at least one carbon-carbon double or triple bond, and this bond is conjugated with a double bond present between C1 and C2, or C2 and C3. In one embodiment, the C₁₋₁₂ alkyl group is a group selected from saturated C₁₋₁₂ alkyl, C₂₋₁₂ alkenyl, C₂₋₁₂ alkynyl and C₃₋₁₂ cycloalkyl.

If a substituent on R² is halo, it is preferably F or C1, more preferably C1. If a substituent on R² is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy).

If a substituent on R² is C₁₋₇ alkyl, it may preferably be a C₁₋₄ alkyl group (e.g. methyl, ethyl, propyl, butyl).

If a substituent on R² is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups.

If a substituent on R² is bis-oxy-C₁₋₃ alkylene, this is preferably bis-oxy-methylene or bis-oxy-ethylene.

Particularly preferred substituents for R² include methoxy, ethoxy, fluoro, chloro, cyano, bis-oxy-methylene, methyl-piperazinyl, morpholino and methyl-thienyl.

Particularly preferred substituted R² groups include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthienyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl.

A particularly preferred unsubstituted R² group is methyl.

In one embodiment, R² is halo or dihalo. In one embodiment, R² is —F or —F₂, which substituents are illustrated below as (III) and (IV) respectively:

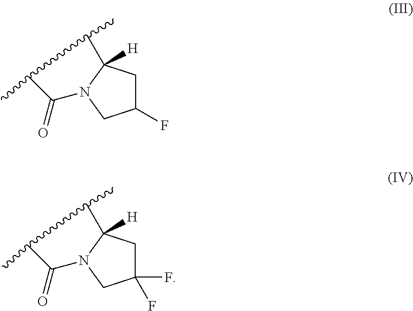

R^{D}

In one embodiment, R^{D} is independently selected from R, CO₂R, COR, CHO, CO₂H, and halo.

In one embodiment, R^{D} is independently R.

In one embodiment, R^{D} is independently halo.

R⁶

In one embodiment, R⁶ is independently selected from H, R, OH, OR, SH, SR, NH₂, NHR, NRR′, NO₂, Me₃Sn— and Halo.

In one embodiment, R⁶ is independently selected from H, OH, OR, SH, NH₂, NO₂ and Halo.

In one embodiment, R⁶ is independently selected from H and Halo.

In one embodiment, R⁶ is independently H.

In one embodiment, R⁶ and R⁷ together form a group —O—(CH₂)ₚ—O—, where p is 1 or 2.

R⁷ is independently selected from H, R, OH, OR, SH, SR, NH₂, NHR, NRR′, NO₂, Me₃Sn and halo.

In one embodiment, R⁷ is independently OR.

In one embodiment, R⁷ is independently OR^{7A}, where R^{7A} is independently optionally substituted C₁₋₆ alkyl.

In one embodiment, R^{7A} is independently optionally substituted saturated C₁₋₆ alkyl.

In one embodiment, R^{7A} is independently optionally substituted C₂₋₄ alkenyl.

In one embodiment, R^{7A} is independently Me.

In one embodiment, R^{7A} is independently CH₂Ph.

In one embodiment, R^{7A} is independently allyl.

In one embodiment, the compound is a dimer where the R⁷ groups of each monomer form together a dimer bridge having the formula X—R″—X linking the monomers.

R⁹

In one embodiment, R⁹ is independently selected from H, R, OH, OR, SH, SR, NH₂, NHR, NRR′, NO₂, Me₃Sn— and Halo.

In one embodiment, R⁹ is independently H.

In one embodiment, R⁹ is independently R or OR.

R and R′

In one embodiment, R is independently selected from optionally substituted C₁₋₁₂ alkyl, C₃₋₂₀ heterocyclyl and C₅₋₂₀ aryl groups. These groups are each defined in the substituents section below.

In one embodiment, R is independently optionally substituted C₁₋₁₂ alkyl.

In one embodiment, R is independently optionally substituted C₃₋₂₀ heterocyclyl.

In one embodiment, R is independently optionally substituted C₅₋₂₀ aryl.

In one embodiment, R is independently optionally substituted C₁₋₁₂ alkyl.

Described above in relation to R² are various embodiments relating to preferred alkyl and aryl groups and the identity and number of optional substituents. The preferences set out for R² as it applies to R are applicable, where appropriate, to all other groups R, for examples where R⁶, R⁷, R⁸ or R⁹ is R.

The preferences for R apply also to R′.

In some embodiments of the invention there is provided a compound having a substituent group —NRR′. In one embodiment, R and R′ together with the nitrogen atom to which they are attached form an optionally substituted 4-, 5-, 6- or 7-membered heterocyclic ring. The ring may contain a further heteroatom, for example N, O or S.

In one embodiment, the heterocyclic ring is itself substituted with a group R. Where a further N heteroatom is present, the substituent may be on the N heteroatom.

R″

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted.

In one embodiment, R″ is a C₃₋₁₂ alkylene group, which chain may be interrupted by one or more heteroatoms and/or aromatic rings, e.g. benzene or pyridine.

In one embodiment, the alkylene group is optionally interrupted by one or more heteroatoms selected from O, S, and NMe and/or aromatic rings, which rings are optionally substituted.

In one embodiment, the aromatic ring is a C₅₋₂₀ arylene group, where arylene pertains to a divalent moiety obtained by removing two hydrogen atoms from two aromatic ring atoms of an aromatic compound, which moiety has from 5 to 20 ring atoms.

In one embodiment, R″ is a C₃₋₁₂ alkylene group, which chain may be interrupted by one or more heteroatoms, e.g. O, S, N(H), NMe and/or aromatic rings, e.g. benzene or pyridine, which rings are optionally substituted by NH₂.

In one embodiment, R″ is a C₃₋₁₂ alkylene group.

In one embodiment, R″ is selected from a C₃, C₅, C₇, C₉ and a C₁₁ alkylene group.

In one embodiment, R″ is selected from a C₃, C₅ and a C₇ alkylene group.

In one embodiment, R″ is selected from a C₃ and a C₅ alkylene group.

In one embodiment, R″ is a C₃ alkylene group.

In one embodiment, R″ is a C₅ alkylene group.

The alkylene groups listed above may be optionally interrupted by one or more heteroatoms and/or aromatic rings, e.g. benzene or pyridine, which rings are optionally substituted.

The alkylene groups listed above may be optionally interrupted by one or more heteroatoms and/or aromatic rings, e.g. benzene or pyridine.

The alkylene groups listed above may be unsubstituted linear aliphatic alkylene groups.

X

In one embodiment, X is selected from O, S, or N(H).

Preferably, X is O.

R¹⁰

The linker attaches the cell binding agent (CBA), to the PBD drug moiety D through covalent bond(s). The linker is a bifunctional or multifunctional moiety which can be used to link one or more drug moiety (D) and a cell binding agent (CBA) to form antibody-drug conjugates (ADC). The linker (L) may be stable outside a cell, i.e. extracellular, or it may be cleavable by enzymatic activity, hydrolysis, or other metabolic conditions. Antibody-drug conjugates (ADC) can be conveniently prepared using a linker having reactive functionality for binding to the drug moiety and to the antibody. A cysteine thiol, or an amine, e.g. N-terminus or amino acid side chain such as lysine, of the antibody (Ab) can form a bond with a functional group of a linker or spacer reagent, PBD drug moiety (D) or drug-linker reagent (D-L).

Many functional groups on the linker attached to the N10 position of the PBD moiety may be useful to react with the cell binding agent. For example, ester, thioester, amide, thioamide, carbamate, thiocarbamate, urea, thiourea, ether, thioether, or disulfide linkages may be formed from reaction of the linker-PBD drug intermediates and the cell binding agent.

The linkers of the ADC preferably prevent aggregation of ADC molecules and keep the ADC freely soluble in aqueous media and in a monomeric state.

The linkers of the ADC are preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targetted site; and (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p 234-242).

In another embodiment, the linker may be substituted with groups which modulate aggregation, solubility or reactivity. For example, a sulfonate substituent may increase water solubility of the reagent and facilitate the coupling reaction of the linker reagent with the antibody or the drug moiety, or facilitate the coupling reaction of Ab-L with D, or D-L with Ab, depending on the synthetic route employed to prepare the ADC.

In one embodiment, R¹⁰ is a group:

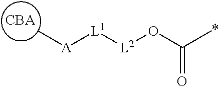

wherein the asterisk indicates the point of attachment to the N10 position, CBA is a cell binding agent/modulator, L¹ is a linker, A is a connecting group connecting L¹ to the cell binding agent, L² is a covalent bond or together with —OC(═O)— forms a self-immolative linker, and L¹ or L² is a cleavable linker.

L¹ is preferably the cleavable linker, and may be referred to as a trigger for activation of the linker for cleavage.

The nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidising conditions may also find use in the present invention.

L¹ may comprise a contiguous sequence of amino acids. The amino acid sequence may be the target substrate for enzymatic cleavage, thereby allowing release of R¹⁰ from the N10 position.

In one embodiment, L¹ is cleavable by the action of an enzyme. In one embodiment, the enzyme is an esterase or a peptidase.

In one embodiment, L² is present and together with —C(═O)O— forms a self-immolative linker. In one embodiment, L² is a substrate for enzymatic activity, thereby allowing release of R¹⁰ from the N10 position.

In one embodiment, where L¹ is cleavable by the action of an enzyme and L² is present, the enzyme cleaves the bond between L¹ and L².

L¹ and L², where present, may be connected by a bond selected from:

—C(═O)NH—,

—C(═O)O—,

—NHC(═O)—,

—OC(═O)—,

—OC(═O)O—,

—NHC(═O)O—,

—OC(═O)NH—, and

—NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

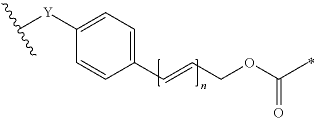

wherein the asterisk indicates the point of attachment to the N10 position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents as described herein. In one embodiment, the phenylene group is optionally substituted with halo, NO₂, R or OR.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

The self-immolative linker will allow for release of the protected compound when a remote site is activated, proceeding along the lines shown below (for n=0):

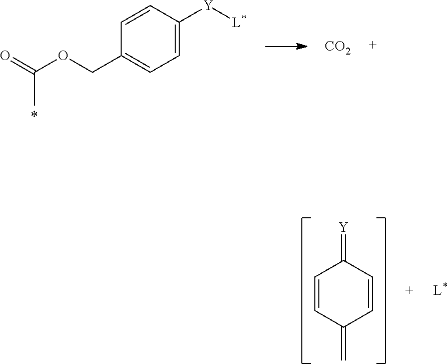

wherein L* is the activated form of the remaining portion of the linker. These groups have the advantage of separating the site of activation from the compound being protected. As described above, the phenylene group may be optionally substituted.

In one embodiment described herein, the group L* is a linker L¹ as described herein, which may include a dipeptide group.

In another embodiment, —C(═O)O— and L² together form a group selected from:

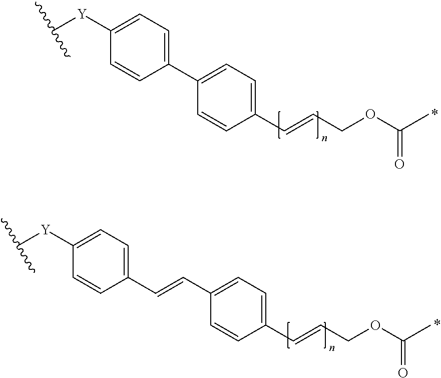

wherein the asterisk, the wavy line, Y, and n are as defined above. Each phenylene ring is optionally substituted with one, two or three substituents as described herein. In one embodiment, the phenylene ring having the Y substituent is optionally substituted and the phenylene ring not having the Y substituent is unsubstituted. In one embodiment, the phenylene ring having the Y substituent is unsubstituted and the phenylene ring not having the Y substituent is optionally substituted.

In another embodiment, —C(═O)O— and L² together form a group selected from:

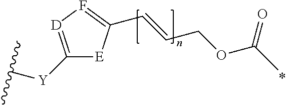

wherein the asterisk, the wavy line, Y, and n are as defined above, E is O, S or NR, D is N, CH, or CR, and F is N, CH, or CR.

In one embodiment, D is N.

In one embodiment, D is CH.

In one embodiment, E is O or S.

In one embodiment, F is CH.

In a preferred embodiment, the linker is a cathepsin labile linker.

In one embodiment, L¹ comprises a dipeptide The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from:

-Phe-Lys-,

-Val-Ala-,

-Val-Lys-,

-Ala-Lys-,

-Val-Cit-,

-Phe-Cit-,

-Leu-Cit-,

-Ile-Cit-,

-Phe-Arg-,

-Trp-Cit-

wherein Cit is citrulline.

Preferably, the group —X₁—X₂— in dipeptide, —NH—X₁—X₂—CO—, is selected from:

-Phe-Lys-,

-Val-Ala-,

-Val-Lys-,

-Ala-Lys-,

-Val-Cit-.

Most preferably, the group —X₁—X₂— in dipeptide, —NH—X₁—X₂—CO—, is -Phe-Lys- or -Val-Ala-.

Other dipeptide combinations may be used, including those described by Dubowchik et al., Bioconjugate Chemistry, 2002, 13, 855-869, which is incorporated herein by reference.

In one embodiment, the amino acid side chain is derivatised, where appropriate. For example, an amino group or carboxy group of an amino acid side chain may be derivatised.

In one embodiment, an amino group NH₂ of a side chain amino acid, such as lysine, is a derivatised form selected from the group consisting of NHR and NRR′.

In one embodiment, a carboxy group COOH of a side chain amino acid, such as aspartic acid, is a derivatised form selected from the group consisting of COOR,

CONH₂, CONHR and CONRR′.

In one embodiment, the amino acid side chain is chemically protected, where appropriate. The side chain protecting group may be a group as discussed below in relation to the group R^{L}. Protected amino acid sequences are cleavable by enzymes. For example, it has been established that a dipeptide sequence comprising a Boc side chain-protected Lys residue is cleavable by cathepsin.

Protecting groups for the side chains of amino acids are well known in the art and are described in the Novabiochem Catalog. Additional protecting group strategies are set out in Protective Groups in Organic Synthesis, Greene and Wuts.

Possible side chain protecting groups are shown below for those amino acids having reactive side chain functionality:

Arg: Z, Mtr, Tos;

Asn: Trt, Xan;

Asp: Bzl, t-Bu;

Cys: Acm, Bzl, Bzl-OMe, Bzl-Me, Trt;

Glu: Bzl, t-Bu;

Gln: Trt, Xan;

His: Boc, Dnp, Tos, Trt;

Lys: Boc, Z—Cl, Fmoc, Z, Alloc;

Ser: Bzl, TBDMS, TBDPS;

Thr: Bz;

Trp: Boc;

Tyr: Bzl, Z, Z—Br.

In one embodiment, the side chain protection is selected to be orthogonal to a group provided as, or as part of, a capping group, where present. Thus, the removal of the side chain protecting group does not remove the capping group, or any protecting group functionality that is part of the capping group.

In other embodiments of the invention, the amino acids selected are those having no reactive side chain functionality. For example, the amino acids may be selected from: Ala, Gly, Ile, Leu, Met, Phe, Pro, and Val.

In one embodiment, the dipeptide is used in combination with a self-immolative linker. The self-immolative linker may be connected to —X₂—.

Where a self-immolative linker is present, —X₂— is connected directly to the self-immolative linker. Preferably the group —X₂—CO— is connected to Y, where Y is NH, thereby forming the group —X₂—CO—NH—.

—NH—X₁— is connected directly to A. A may comprise the functionality —CO— thereby to form an amide link with —X₁—.

In one embodiment, L¹ and L² together with —OC(═O)— comprise the group NH—X₁—X₂—CO-PABC—. The PABC group is connected directly to the N10 position. Preferably, the self-immolative linker and the dipeptide together form the group —NH-Phe-Lys-CO—NH-PABC—, which is illustrated below:

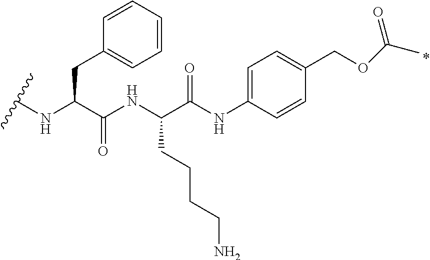 [SMILES: *N[C@@H](Cc1ccccc1)C(=O)N[C@@H](CCCCN)C(=O)Nc1ccc(COC(C)=O)cc1]

wherein the asterisk indicates the point of attachment to the N10 position, and the wavy line indicates the point of attachment to the remaining portion of the linker L′ or the point of attachment to A. Preferably, the wavy line indicates the point of attachment to A. The side chain of the Lys amino acid may be protected, for example, with Boc, Fmoc, or Alloc, as described above.

Alternatively, the self-immolative linker and the dipeptide together form the group —NH-Val-Ala-CO—NH-PABC—, which is illustrated below:

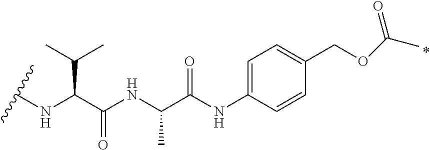 [SMILES: *N[C@H](C(=O)N[C@@H](C)C(=O)Nc1ccc(COC(C)=O)cc1)C(C)C]

wherein the asterisk and the wavy line are as defined above.

Alternatively, the self-immolative linker and the dipeptide together form the group —NH-Val-Cit-CO—NH-PABC—, which is illustrated below:

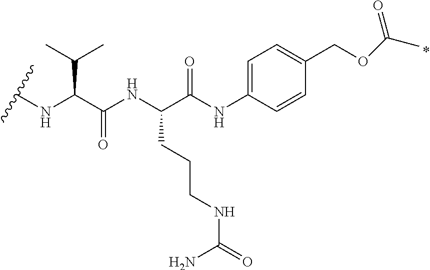 [SMILES: *N[C@H](C(=O)N[C@@H](CCCNC(N)=O)C(=O)Nc1ccc(COC(C)=O)cc1)C(C)C]

wherein the asterisk and the wavy line are as defined above.

In some embodiments of the present invention, it may be preferred that if the PBD/drug moiety contains an unprotected imine bond, e.g. if moiety B is present, then the linker does not contain a free amino (H₂N—) group. Thus if the linker has the structure -A-L¹-L²- then this would preferably not contain a free amino group. This preference is particularly relevant when the linker contains a dipeptide, for example as L′; in this embodiment, it would be preferred that one of the two amino acids is not selected from lysine.

Without wishing to be bound by theory, the combination of an unprotected imine bond in the drug moiety and a free amino group in the linker can cause dimerisation of the drug-linker moiety which may interfere with the conjugation of such a drug-linker moiety to an antibody. The cross-reaction of these groups may be accelerated in the case the free amino group is present as an ammonium ion (H₃N⁺—), such as when a strong acid (e.g. TFA) has been used to deprotect the free amino group.

In one embodiment, A is a covalent bond. Thus, L¹ and the cell binding agent are directly connected. For example, where L¹ comprises a contiguous amino acid sequence, the N-terminus of the sequence may connect directly to the cell binding agent.

Thus, where A is a covalent bond, the connection between the cell binding agent and L¹ may be selected from:

—C(═O)NH—,

—C(═O)O—,

—NHC(═O)—,

—OC(═O)—,

—OC(═O)O—,

—NHC(═O)O—,

—OC(═O)NH—,

—NHC(═O)NH—,

—C(═O)NHC(═O)—,

—S—,

—S—S—,

—CH₂C(═O)—, and

═N—NH—.

An amino group of L¹ that connects to the cell binding agent may be the N-terminus of an amino acid or may be derived from an amino group of an amino acid side chain, for example a lysine amino acid side chain.

An carboxyl group of L¹ that connects to the cell binding agent may be the C-terminus of an amino acid or may be derived from a carboxyl group of an amino acid side chain, for example a glutamic acid amino acid side chain.

A hydroxyl group of L¹ that connects to the cell binding agent may be derived from a hydroxyl group of an amino acid side chain, for example a serine amino acid side chain.

A thiol group of L¹ that connects to the cell binding agent may be derived from a thiol group of an amino acid side chain, for example a serine amino acid side chain.

The comments above in relation to the amino, carboxyl, hydroxyl and thiol groups of L¹ also apply to the cell binding agent.

In one embodiment, L² together with —OC(═O)— represents:

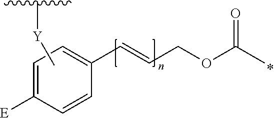

wherein the asterisk indicates the point of attachment to the N10 position, the wavy line indicates the point of attachment to L¹, n is 0 to 3, Y is a covalent bond or a functional group, and E is an activatable group, for example by enzymatic action or light, thereby to generate a self-immolative unit. The phenylene ring is optionally further substituted with one, two or three substituents as described herein. In one embodiment, the phenylene group is optionally further substituted with halo, NO₂, R or OR. Preferably n is 0 or 1, most preferably 0.

E is selected such that the group is susceptible to activation, e.g. by light or by the action of an enzyme. E may be —NO₂ or glucoronic acid. The former may be susceptible to the action of a nitroreductase, the latter to the action of a β-glucoronidase.

In this embodiment, the self-immolative linker will allow for release of the protected compound when E is activated, proceeding along the lines shown below (for n=0):

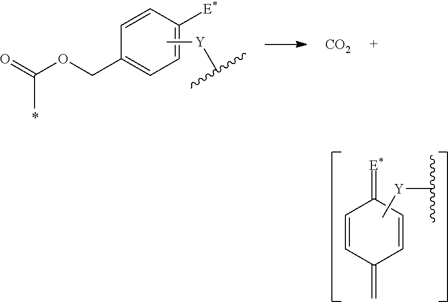

wherein the asterisk indicates the point of attachment to the N10 position, E* is the activated form of E, and Y is as described above. These groups have the advantage of separating the site of activation from the compound being protected. As described above, the phenylene group may be optionally further substituted.

The group Y may be a covalent bond to L′.

The group Y may be a functional group selected from:

—C(═O)—

—NH—

—O—

—C(═O)NH—,

—C(═O)O—,

—NHC(═O)—,

—OC(═O)—,

—OC(═O)O—,

—NHC(═O)O—,

—OC(═O)NH—,

—NHC(═O)NH—,

—NHC(═O)NH,

—C(═O)NHC(═O)—, and

—S—.

Where L¹ is a dipeptide, it is preferred that Y is —NH— or —C(═O)—, thereby to form an amide bond between L¹ and Y. In this embodiment, the dipeptide sequence need not be a substrate for an enzymatic activity.

In another embodiment, A is a spacer group. Thus, L¹ and the cell binding agent are indirectly connected.

L¹ and A may be connected by a bond selected from:
—C(═O)NH—,
—C(═O)O—,
—NHC(═O)—,
—OC(═O)—,
—OC(═O)O—,
—NHC(═O)O—,
—OC(═O)NH—, and
—NHC(═O)NH—.

Preferably, the linker contains an electrophilic functional group for reaction with a nucleophilic functional group on the cell binding agent. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones, carboxyl, and, some of which are exemplified as follows:

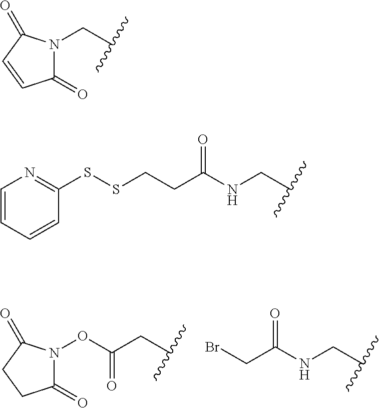 [SMILES: *CN1C(=O)C=CC1=O ; *CNC(=O)CCSSc1ccccn1 ; *CC(=O)ON1C(=O)CCC1=O ; *CNC(=O)CBr]

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothio lane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, a Linker has a reactive nucleophilic group which is reactive with an electrophilic group present on an antibody. Useful electrophilic groups on an antibody include, but are not limited to, aldehyde and ketone carbonyl groups. The heteroatom of a nucleophilic group of a Linker can react with an electrophilic group on an antibody and form a covalent bond to an antibody unit. Useful nucleophilic groups on a Linker include, but are not limited to, hydrazide, oxime, amino, hydroxyl, hydrazine, thiosemicarbazone, hydrazine carboxylate, and arylhydrazide. The electrophilic group on an antibody provides a convenient site for attachment to a Linker.

In one embodiment, the group A is:

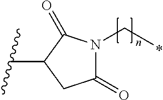

wherein the asterisk indicates the point of attachment to L¹, the wavy line indicates the point of attachment to the cell binding agent, and n is 0 to 6. In one embodiment, n is 5.

In one embodiment, the group A is:

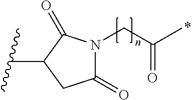

where the asterisk indicates the point of attachment to L¹, the wavy line indicates the point of attachment to the cell binding agent, and n is 0 to 6. In one embodiment, n is 5.

In one embodiment, the group A is:

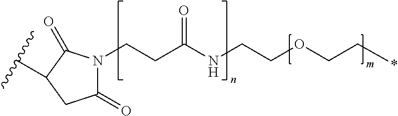

wherein the asterisk indicates the point of attachment to L¹, the wavy line indicates the point of attachment to the cell binding agent, n is 0 or 1, and m is 0 to 30. In a preferred embodiment, n is 1 and m is 0 to 10, 1 to 8, preferably 4 to 8, and most preferably 4 or 8. In another embodiment, m is 10 to 30, and preferably 20 to 30. Alternatively, m is 0 to 50. In this embodiment, m is preferably 10-40 and n is 1.

In one embodiment, the group A is:

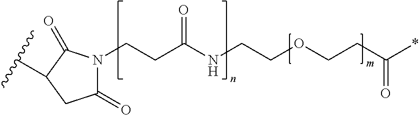

wherein the asterisk indicates the point of attachment to L¹, the wavy line indicates the point of attachment to the cell binding agent, n is 0 or 1, and m is 0 to 30. In a preferred embodiment, n is 1 and m is 0 to 10, 1 to 8, preferably 4 to 8, and most preferably 4 or 8. In another embodiment, m is 10 to 30, and preferably 20 to 30. Alternatively, m is 0 to 50. In this embodiment, m is preferably 10-40 and n is 1.

In one embodiment, the connection between the cell binding agent and A is through a thiol residue of the cell binding agent and a maleimide group of A.

In one embodiment, the connection between the cell binding agent and A is:

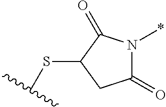 [SMILES: *SC1CC(=O)N(*)C1=O]

wherein the asterisk indicates the point of attachment to the remaining portion of A and the wavy line indicates the point of attachment to the remaining portion of the cell binding agent. In this embodiment, the S atom is typically derived from the cell binding agent.

In each of the embodiments above, an alternative functionality may be used in place of the maleimide-derived group shown below:

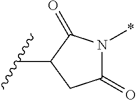 [SMILES: *C1CC(=O)N(*)C1=O]

wherein the wavy line indicates the point of attachment to the cell binding agent as before, and the asterisk indicates the bond to the remaining portion of the A group.

In one embodiment, the maleimide-derived group is replaced with the group:

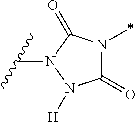 [SMILES: *n1[nH]c(=O)n(*)c1=O]

wherein the wavy line indicates point of attachment to the cell binding agent, and the asterisk indicates the bond to the remaining portion of the A group.

In one embodiment, the maleimide-derived group is replaced with a group, which optionally together with the cell binding agent, is selected from:

—C(═O)NH—,

—C(═O)O—,

—NHC(═O)—,

—OC(═O)—,

—OC(═O)O—,

—NHC(═O)O—,

—OC(═O)NH—,

—NHC(═O)NH—,

—NHC(═O)NH,

—C(═O)NHC(═O)—,

—S—,

—S—S—,

—CH₂C(═O)—

—C(═O)CH₂—,

═N—NH—, and

—NH—N═.

In one embodiment, the maleimide-derived group is replaced with a group, which optionally together with the cell binding agent, is selected from:

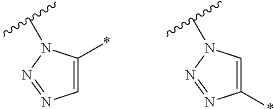 [SMILES: *c1cnnn1* ; *c1cn(*)nn1]

wherein the wavy line indicates either the point of attachment to the cell binding agent or the bond to the remaining portion of the A group, and the asterisk indicates the other of the point of attachment to the cell binding agent or the bond to the remaining portion of the A group.

Other groups suitable for connecting L¹ to the cell binding agent are described in WO 2005/082023.

The group R^{C} is removable from the N10 position of the PBD moiety to leave an N10-C11 imine bond, a carbinolamine, a substituted carbinolamine, where QR¹¹ is OSO₃M, a bisulfite adduct, a thiocarbinolamine, a substituted thiocarbinolamine, or a substituted carbinalamine.

In one embodiment, R^{C}, may be a protecting group that is removable to leave an N10-C11 imine bond, a carbinolamine, a substituted cabinolamine, or, where QR¹¹ is OSO₃M, a bisulfite adduct. In one embodiment, R^{C} is a protecting group that is removable to leave an N10-C11 imine bond.

The group R^{C} is intended to be removable under the same conditions as those required for the removal of the group R¹⁰, for example to yield an N10-C11 imine bond, a carbinolamine and so on. The capping group acts as a protecting group for the intended functionality at the N10 position. The capping group is intended not to be reactive towards a cell binding agent. For example, R^{C} is not the same as R^{L}.

Compounds having a capping group may be used as intermediates in the synthesis of dimers having an imine monomer. Alternatively, compounds having a capping group may be used as conjugates, where the capping group is removed at the target location to yield an imine, a carbinolamine, a substituted cabinolamine and so on.

Thus, in this embodiment, the capping group may be referred to as a therapeutically removable nitrogen protecting group, as defined in WO 00/12507.

In one embodiment, the group R^{C} is removable under the conditions that cleave the linker R^{L} of the group R¹⁰. Thus, in one embodiment, the capping group is cleavable by the action of an enzyme.

In an alternative embodiment, the capping group is removable prior to the connection of the linker R^{L} to the cell binding agent. In this embodiment, the capping group is removable under conditions that do not cleave the linker R^{L}.

Where a compound includes a functional group G¹ to form a connection to the cell binding agent, the capping group is removable prior to the addition or unmasking of G¹.

The capping group may be used as part of a protecting group strategy to ensure that only one of the monomer units in a dimer is connected to a cell binding agent.

The capping group may be used as a mask for a N10-C11 imine bond. The capping group may be removed at such time as the imine functionality is required in the compound. The capping group is also a mask for a carbinolamine, a substituted cabinolamine, and a bisulfite adduct, as described above.

In one embodiment, R^{C} is a carbamate protecting group.

In one embodiment, the carbamate protecting group is selected from: Alloc, Fmoc, Boc, Troc, Teoc, Psec, Cbz and PNZ.

Optionally, the carbamate protecting group is further selected from Moc.

In one embodiment, R^{C} is a linker group R^{L} lacking the functional group for connection to the cell binding agent.

This application is particularly concerned with those R^{C} groups which are carbamates.

In one embodiment, R^{C} is a group:

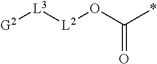

wherein the asterisk indicates the point of attachment to the N10 position, G² is a terminating group, L³ is a covalent bond or a cleavable linker L¹, L² is a covalent bond or together with OC(═O) forms a self-immolative linker.

Where L³ and L² are both covalent bonds, G² and OC(═O) together form a carbamate protecting group as defined above.

L¹ is as defined above in relation to R¹⁰.

L² is as defined above in relation to R¹⁰.

Various terminating groups are described below, including those based on well known protecting groups.

In one embodiment L³ is a cleavable linker L¹, and L², together with OC(═O), forms a self-immolative linker. In this embodiment, G² is Ac (acetyl) or Moc, or a carbamate protecting group selected from: Alloc, Fmoc, Boc, Troc, Teoc, Psec, Cbz and PNZ.

Optionally, the carbamate protecting group is further selected from Moc.

In another embodiment, G² is an acyl group —C(═O)G³, where G³ is selected from alkyl (including cycloalkyl, alkenyl and alkynyl), heteroalkyl, heterocyclyl and aryl (including heteroaryl and carboaryl). These groups may be optionally substituted. The acyl group together with an amino group of L³ or L², where appropriate, may form an amide bond. The acyl group together with a hydroxy group of L³ or L², where appropriate, may form an ester bond.

In one embodiment, G³ is heteroalkyl. The heteroalkyl group may comprise polyethylene glycol. The heteroalkyl group may have a heteroatom, such as O or N, adjacent to the acyl group, thereby forming a carbamate or carbonate group, where appropriate, with a heteroatom present in the group L³ or L², where appropriate.

In one embodiment, G³ is selected from NH₂, NHR and NRR′. Preferably, G³ is NRR′.

In one embodiment G² is the group:

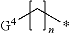

wherein the asterisk indicates the point of attachment to L³, n is 0 to 6 and G⁴ is selected from OH, OR, SH, SR, COOR, CONH₂, CONHR, CONRR′, NH₂, NHR, NRR′, NO₂, and halo. The groups OH, SH, NH₂ and NHR are protected. In one embodiment, n is 1 to 6, and preferably n is 5. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Most preferably G⁴ is OMe.

In one embodiment, the group G² is:

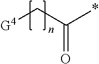

wherein the asterisk indicates the point of attachment to L³, and n and G⁴ are as defined above.

In one embodiment, the group G² is:

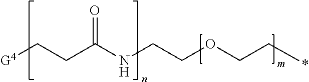

wherein the asterisk indicates the point of attachment to L³, n is 0 or 1, m is 0 to 50, and G⁴ is selected from OH, OR, SH, SR, COOR, CONH₂, CONHR, CONRR′, NH₂, NHR, NRR′, NO₂, and halo. In a preferred embodiment, n is 1 and m is 0 to 10, 1 to 2, preferably 4 to 8, and most preferably 4 or 8. In another embodiment, n is 1 and m is 10 to 50, preferably 20 to 40. The groups OH, SH, NH₂ and NHR are protected. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Preferably G⁴ is OMe.

In one embodiment, the group G² is:

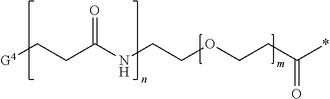

wherein the asterisk indicates the point of attachment to L³, and n, m and G⁴ are as defined above.

In one embodiment, the group G² is:

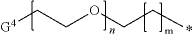

wherein n is 1-20, m is 0-6, and G⁴ is selected from OH, OR, SH, SR, COOR, CONH₂, CONHR, CONRR′, NH₂, NHR, NRR′, NO₂, and halo. In one embodiment, n is 1-10. In another embodiment, n is 10 to 50, preferably 20 to 40. In one embodiment, n is 1. In one embodiment, m is 1. The groups OH, SH, NH₂ and NHR are protected. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Preferably G⁴ is OMe.

In one embodiment, the group G² is:

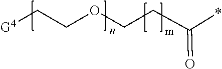

wherein the asterisk indicates the point of attachment to L³, and n, m and G⁴ are as defined above.

In each of the embodiments above G⁴ may be OH, SH, NH₂ and NHR. These groups are preferably protected.

In one embodiment, OH is protected with Bzl, TBDMS, or TBDPS.

In one embodiment, SH is protected with Acm, Bzl, Bzl-OMe, Bzl-Me, or Trt.

In one embodiment, NH₂ or NHR are protected with Boc, Moc, Z—Cl, Fmoc, Z, or Alloc.

In one embodiment, the group G² is present in combination with a group L³, which group is a dipeptide.

The capping group is not intended for connection to the cell binding agent. Thus, the other monomer present in the dimer serves as the point of connection to the cell binding agent via a linker. Accordingly, it is preferred that the functionality present in the capping group is not available for reaction with a cell binding agent. Thus, reactive functional groups such as OH, SH, NH₂, COOH are preferably avoided. However, such functionality may be present in the capping group if protected, as described above.

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Isomers

Certain compounds of the invention may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

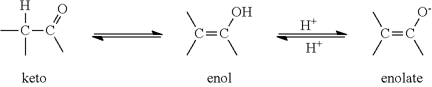

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, and ¹⁴C; O may ¹⁸F, ³¹P, ³²P, ³⁵S, ³⁶Cl, and ¹²⁵I. Various isotopically labeled compounds of the present invention, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this invention any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

III—The Antibody Conjugate (ADC)

C2 Alkylene

In one embodiment, the conjugate is a compound:

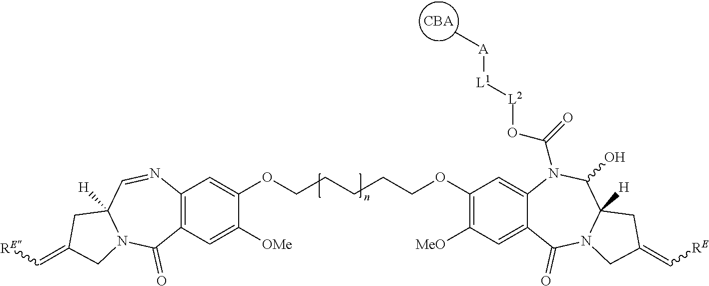

wherein CBA is a cell binding agent as defined above, and n is 0 or 1. L¹ and L² are as previously defined, and R^{E} and R^{E}″ are each independently selected from H or R^{D}.

In one embodiment, the conjugate is a compound:

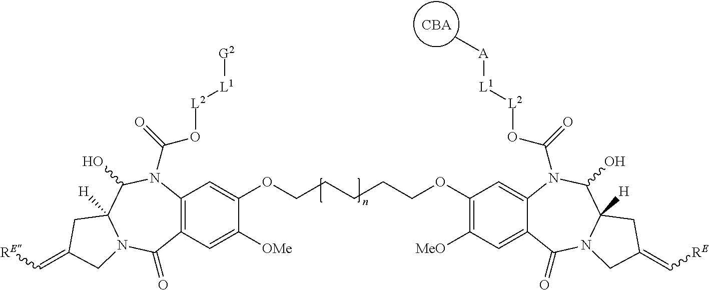

wherein CBA is a cell binding agent as defined above, and n is 0 or 1. L¹, L² and G² are as previously defined, and R^{E} and R^{E}″ are each independently selected from H or R^{D}.

In one embodiment, the conjugate is a compound:

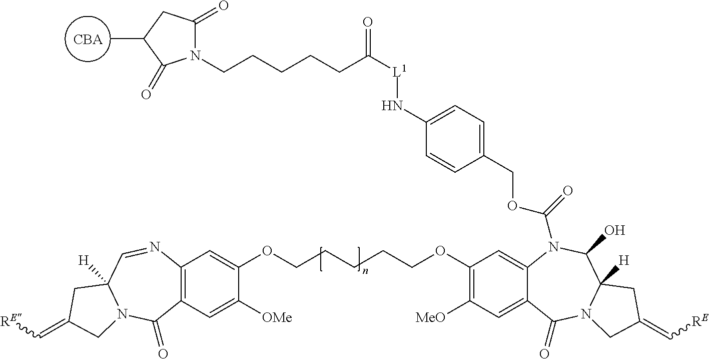

wherein CBA is a cell binding agent as defined above, and n is 0 or 1. L¹ is as previously defined, and R^{E} and R^{E}″ are each independently selected from H or R^{D}.

In one embodiment, the conjugate is a compound:

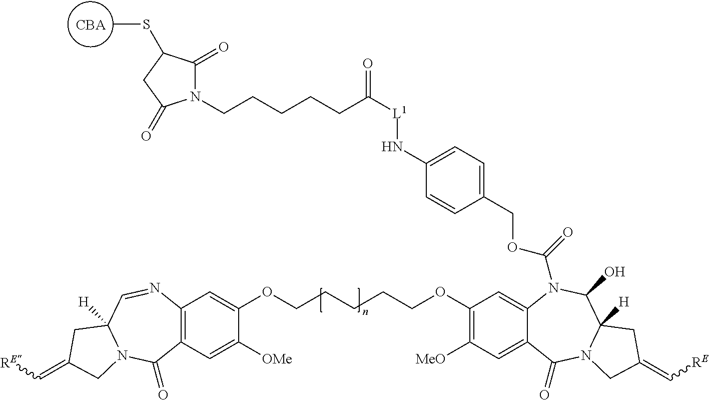

wherein CBA is a cell binding agent as defined above, and n is 0 or 1. L¹ is as previously defined, and R^{E} and R^{E}″ are each independently selected from H or R^{D}.

In one embodiment, the conjugate is a compound:

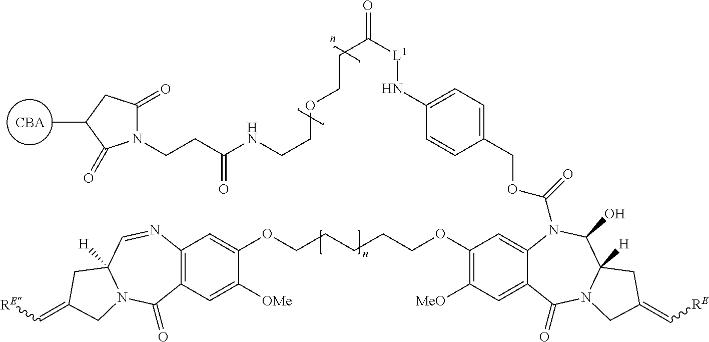

wherein CBA is a cell binding agent as defined above, and n is 0 or 1. L¹ is as previously defined, and R^{E} and R^{E}″ are each independently selected from H or R^{D}.

In one embodiment, the conjugate is a compound:

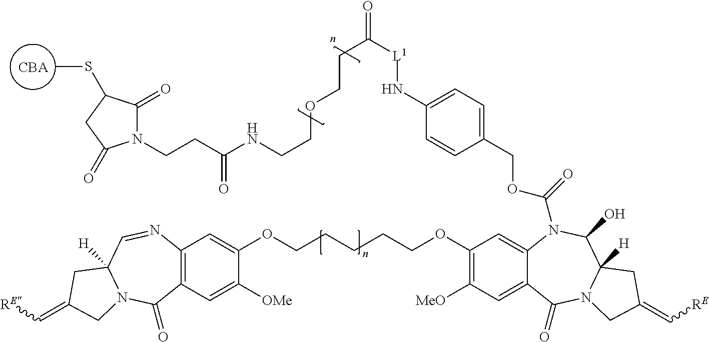

wherein CBA is a cell binding agent as defined above, and n is 0 or 1. L¹ is as previously defined, and R^{E} and R^{E}″ are each independently selected from H or R^{D}.

For each of the compounds above, the following preferences may apply, where appropriate:

n is 0;

n is 1;

R^{E} is H;

R^{E} is R^{D}, where R^{D} is optionally substituted alkyl;

R^{E} is R^{D}, where R^{D} is methyl;

L¹ is or comprises a dipeptide;

L¹ is (H₂N)-Val-Ala-(CO) or (H₂N)-Phe-Lys-(CO), where (H₂N) and (CO) indicate the respective N and C terminals;

L² is p-aminobenzylene;

G² is selected from Alloc, Fmoc, Boc, Troc, Teoc, Psec, Cbz and PNZ.

The following preferences may also apply in addition to the preferences above:

G² is:

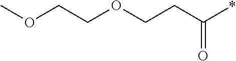 [SMILES: *C(=O)CCOCCOC]

where the asterisk indicates the point of attachment to the N terminal of L′;

A is:

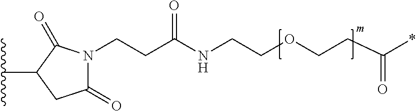

wherein the asterisk indicates the point of attachment to the N terminal of L¹, the wavy line indicates the point of attachment to the cell binding agent and m is 4 or 8;

A is

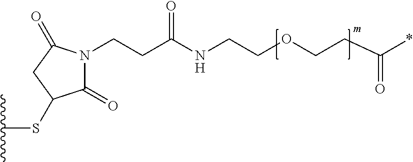

wherein the asterisk indicates the point of attachment to the N terminal of L¹, the wavy line indicates the point of attachment to the cell binding agent, and m is 4 or 8.

In a particularly preferred embodiment, n is 1; R^{E} is H; CBA is an antibody; L′ is (H₂N)-Val-Ala-(CO) or (H₂N)-Phe-Lys-(CO), where (H₂N) and (CO) indicate the respective N and C terminals; L² is p-aminobenzylene; G² is:

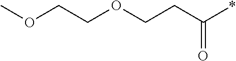 [SMILES: *C(=O)CCOCCOC]

wherein the asterisk indicates the point of attachment to the N terminal of L′; and A is

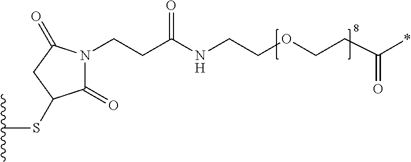

wherein the asterisk indicates the point of attachment to the N terminal of L¹, and the wavy line indicates the point of attachment to the cell binding agent.

C2 Aryl

In one embodiment, the conjugate is a compound:

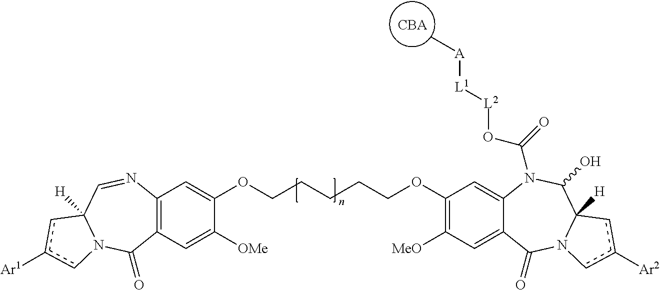

wherein CBA is a cell binding agent as defined above, L¹ and L² are as previously defined Ar¹ and Ar² are each independently optionally substituted C₅₋₂₀ aryl, and n is 0 or 1. Ar¹ and Ar² may be the same or different.

In one embodiment, the conjugate is a compound:

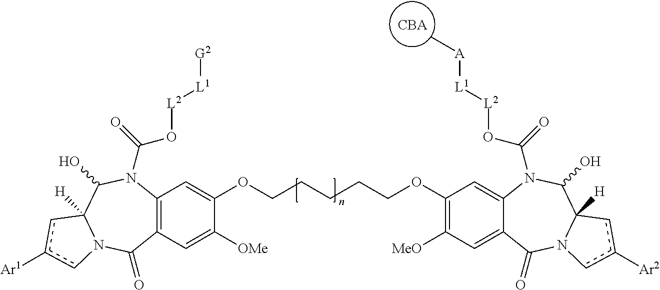

wherein CBA is a cell binding agent as defined above, L¹, L² and G² are as previously defined, Ar¹ and Ar² are each independently optionally substituted C₅₋₂₀ aryl, and n is 0 or 1.

In one embodiment, the conjugate is a compound:

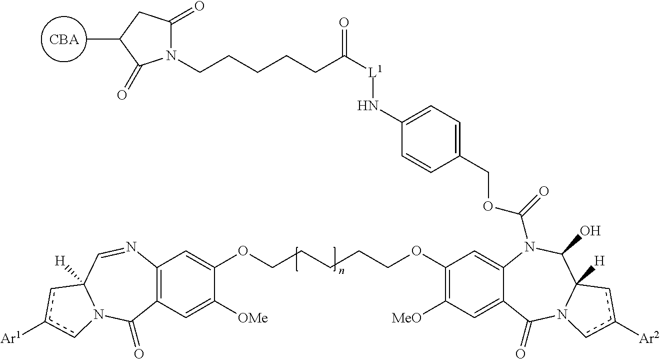

wherein CBA is a cell binding agent as defined above, L¹ is as previously defined, Ar¹ and Ar² are each independently optionally substituted C₅₋₂₀ aryl, and n is 0 or 1.

In one embodiment, the conjugate is a compound:

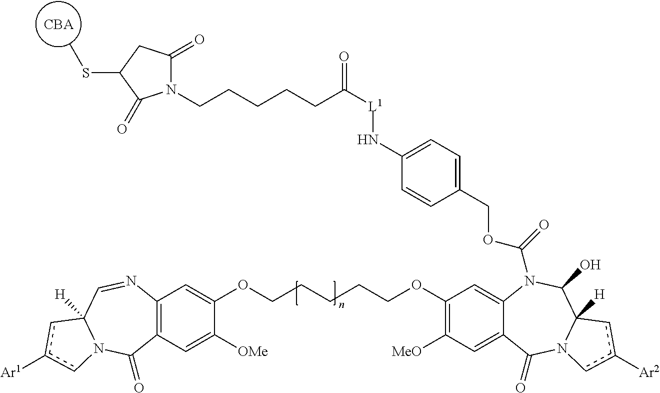

wherein CBA is a cell binding agent as defined above, L¹ is as previously defined, Ar¹ and Ar² are each independently optionally substituted C₅₋₂₀ aryl, and n is 0 or 1.

In one embodiment, the conjugate is a compound:

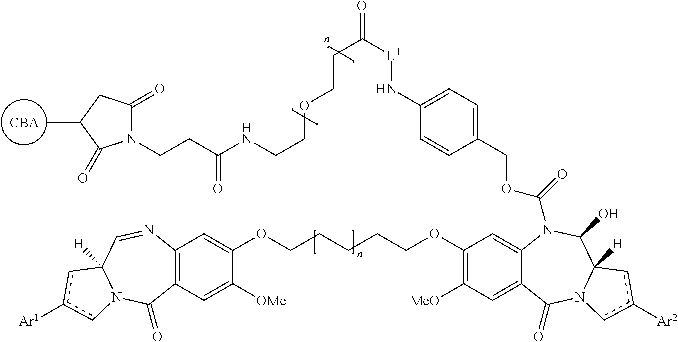

wherein cell binding agent as defined above, and n is 0 or 1. L¹ is as previously defined, Ar¹ and Ar² are each independently optionally substituted C₅₋₂₀ aryl, and n is 0 or 1.

In one embodiment, the conjugate is a compound:

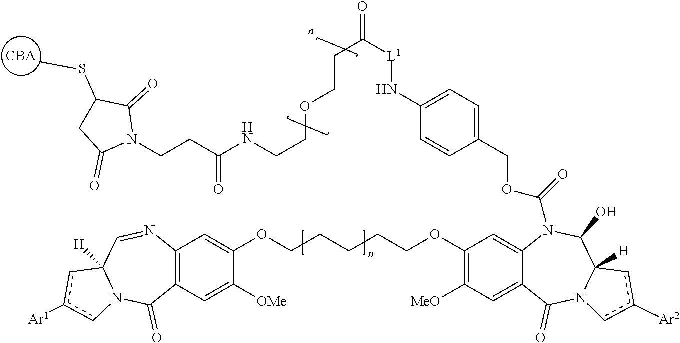

wherein CBA is a cell binding agent as defined above, and n is 0 or 1. L¹ is as previously defined, Ar¹ and Ar² are each independently optionally substituted C₅₋₂₀ aryl, and n is 0 or 1.

In one embodiment, Ar¹ and Ar² in each of the embodiments above are each independently selected from optionally substituted phenyl, furanyl, thiophenyl and pyridyl.

In one embodiment, Ar¹ and Ar² in each of the embodiments above is optionally substituted phenyl.

In one embodiment, Ar¹ and Ar² in each of the embodiments above is optionally substituted thiophen-2-yl or thiophen-3-yl.

In one embodiment, Ar¹ and Ar² in each of the embodiments above is optionally substituted quinolinyl or isoquinolinyl.

The quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. Of these quinolin-3-yl and quinolin-6-yl may be preferred. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. Of these isoquinolin-3-yl and isoquinolin-6-yl may be preferred.

C2 Vinyl

In one embodiment, the conjugate is a compound:

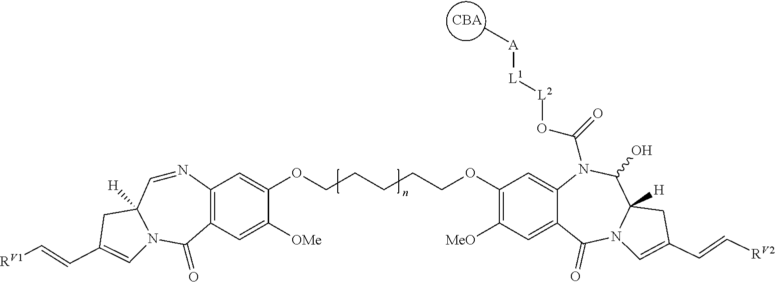

wherein CBA is a cell binding agent as defined above, L¹ and L² are as previously defined, R^{V1} and R^{V2} are independently selected from H, methyl, ethyl and phenyl (which phenyl may be optionally substituted with fluoro, particularly in the 4 position) and C₅₋₆ heterocyclyl, and n is 0 or 1. R^{V1} and R^{V2} may be the same or different.

In one embodiment, the conjugate is a compound:

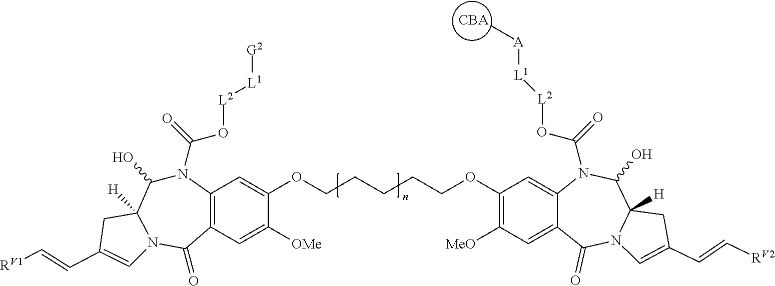

wherein CBA is a cell binding agent as defined above, L¹, L² and G² are as previously defined, R^{V1} and R^{V2} are independently selected from H, methyl, ethyl and phenyl (which phenyl may be optionally substituted with fluoro, particularly in the 4 position) and C₅₋₆ heterocyclyl, and n is 0 or 1. R^{V1} and R^{V2} may be the same or different.

In one embodiment, the conjugate is a compound:

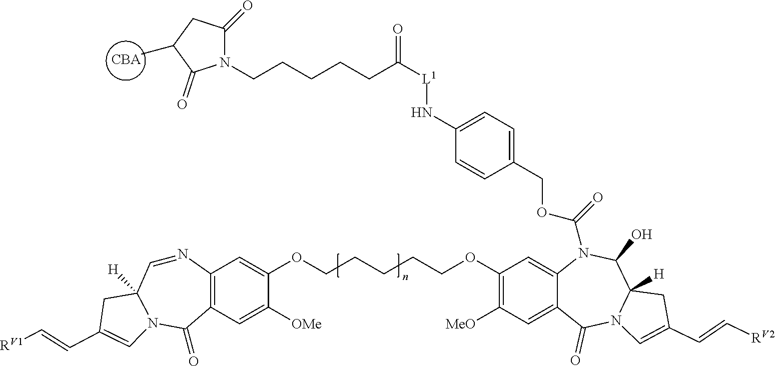

wherein CBA is a cell binding agent as defined above, L¹ is as previously defined, R^{V1} and R^{V2} are independently selected from H, methyl, ethyl and phenyl (which phenyl may be optionally substituted with fluoro, particularly in the 4 position) and C₅₋₆ heterocyclyl, and n is 0 or 1. R^{V1} and R^{V2} may be the same or different.

In one embodiment, the conjugate is a compound:

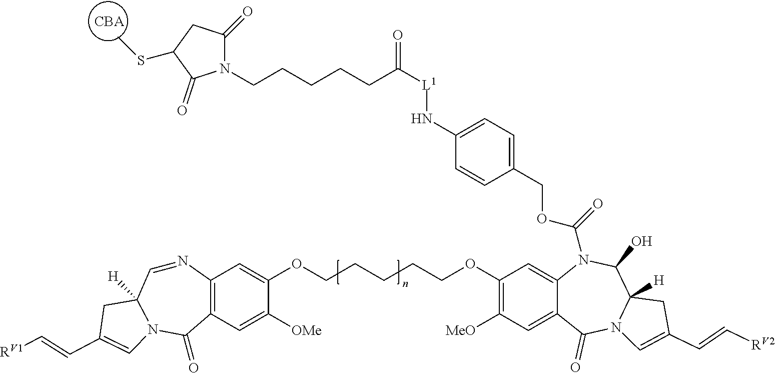

wherein CBA is a cell binding agent as defined above, L¹ is as previously defined, R^{V1} and R^{V2} are independently selected from H, methyl, ethyl and phenyl (which phenyl may be optionally substituted with fluoro, particularly in the 4 position) and C₅₋₆ heterocyclyl, and n is 0 or 1. R^{V1} and R^{V2} may be the same or different.

In one embodiment, the conjugate is a compound:

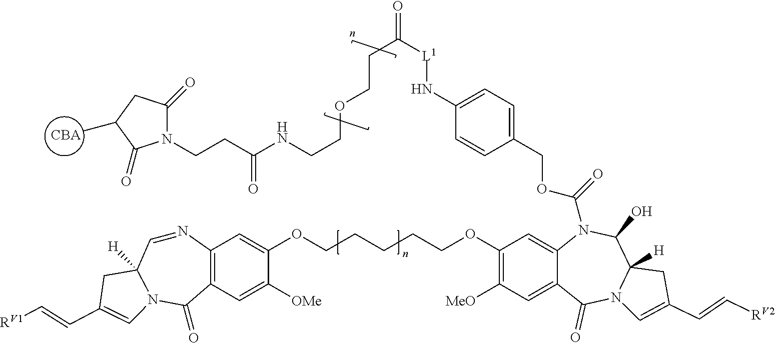

wherein CBA is a cell binding agent as defined above, and n is 0 or 1. L¹ is as previously defined, R^{V1} and R^{V2} are independently selected from H, methyl, ethyl and phenyl (which phenyl may be optionally substituted with fluoro, particularly in the 4 position) and C₅₋₆ heterocyclyl, and n is 0 or 1. R^{V1} and R^{V2} may be the same or different.

In one embodiment, the conjugate is a compound:

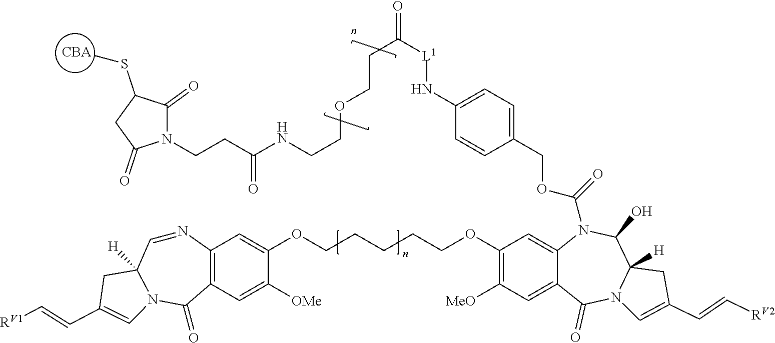

wherein CBA is a cell binding agent as defined above, and n is 0 or 1. L¹ is as previously defined, R^{V1} and R^{V2} are independently selected from H, methyl, ethyl and phenyl (which phenyl may be optionally substituted with fluoro, particularly in the 4 position) and C₅₋₆ heterocyclyl, and n is 0 or 1. R^{V1} and R^{V2} may be the same or different.

In some of the above embodiments, R^{V1} and R^{V2} may be independently selected from H, phenyl, and 4-fluorophenyl.

In a preferred embodiment, the drug D of the ADC of the present invention is selected from:

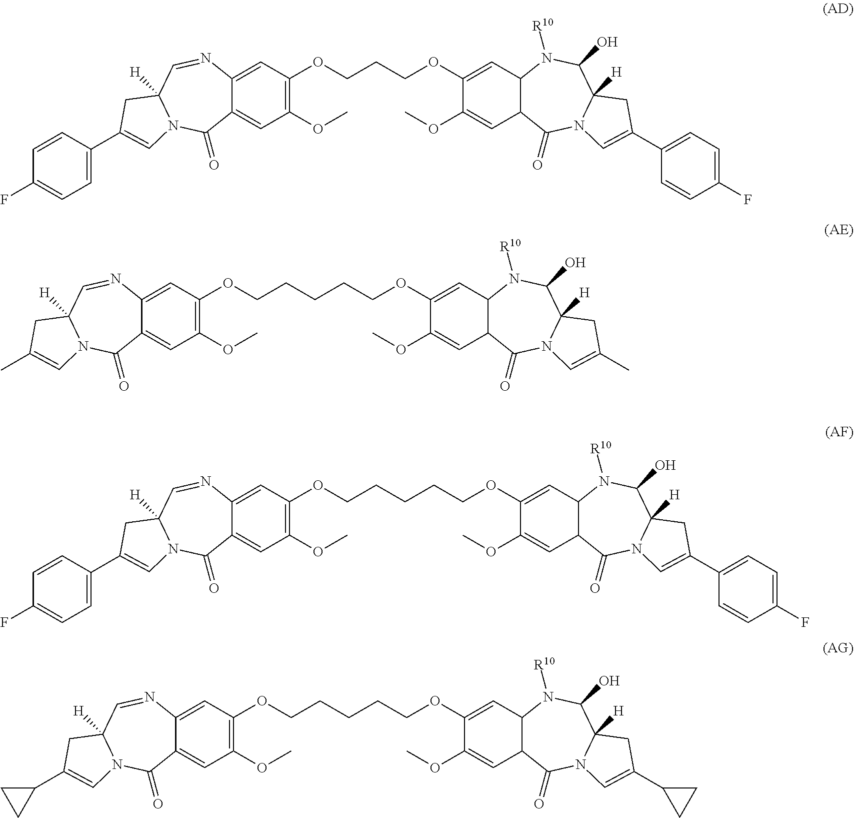

In a preferred embodiment, the ADC of the invention is of the structural general formula:

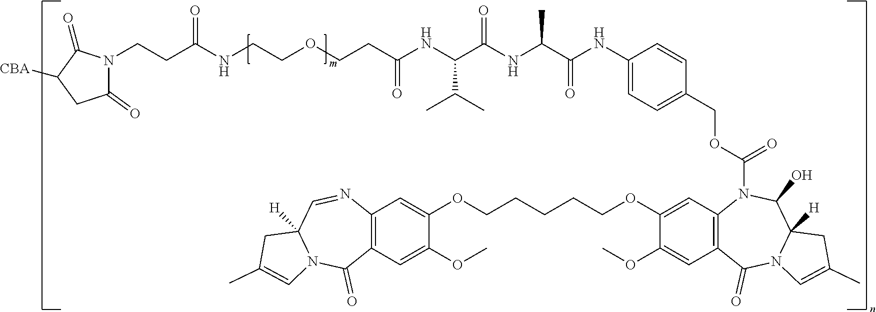

wherein CBA consists of 1613F12, or an antigen binding fragment thereof, m is 0 to 30, and n is 1 to 12.

In a preferred embodiment, the ADC of the invention is of the structural general formula:

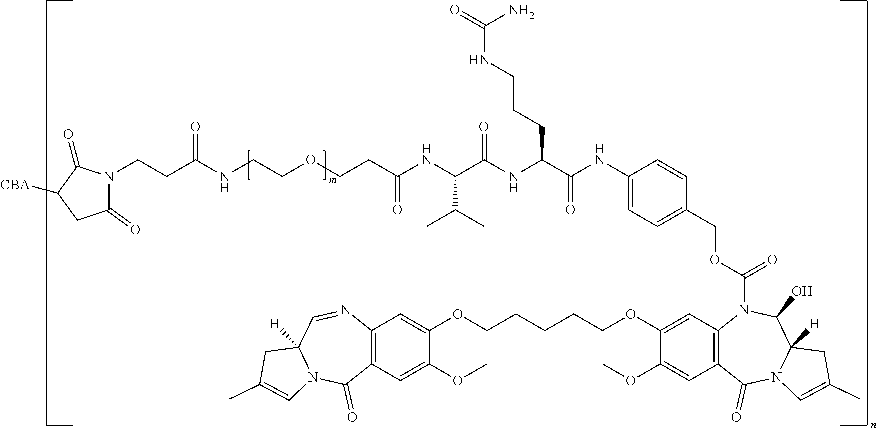

wherein CBA consists of 1613F12, or an antigen binding fragment thereof, m is 0 to 30, and n is 1 to 12.

In a preferred embodiment, the ADC of the invention is of the structural general formula:

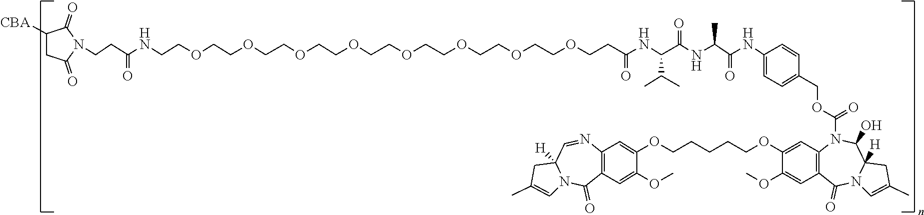

wherein CBA consists of 1613F12, or an antigen binding fragment thereof, and n is 1 to 12.

In a preferred embodiment, the ADC of the invention is of the structural general formula:

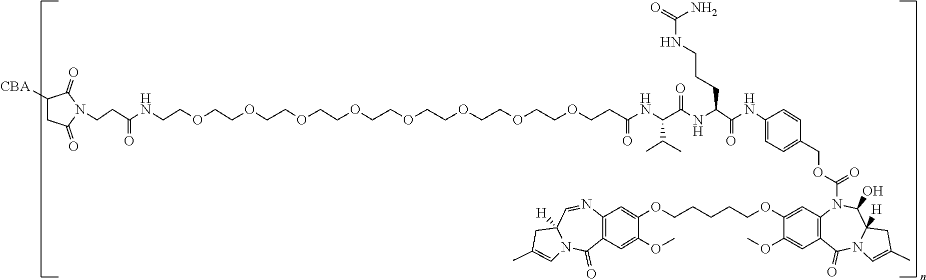

wherein CBA consists of 1613F12, or an antigen binding fragment thereof, and n is 1 to 12.

The drug loading also referred as the Drug-Antibody ratio (DAR) is the average number of PBD drugs per cell binding agent.

In the case of an antibody IgG1 isotype, where the drugs are bound to cysteines after partial antibody reduction, drug loading may range from 1 to 8 drugs (D) per antibody, i.e. where 1, 2, 3, 4, 5, 6, 7, and 8 drug moieties are covalently attached to the antibody.

In the case of an antibody IgG2 isotype, where the drugs are bound to cysteines after partial antibody reduction, drug loading may range from 1 to 12 drugs (D) per antibody, i.e. where 1, 2, 3, 4, 5, 6, 7, 8, 9, 10, 11 and 12 drug moieties are covalently attached to the antibody.

Compositions of ADC include collections of cell binding agents, e.g. antibodies, conjugated with a range of drugs, from 1 to 8 or 1 to 12.

Where the compounds of the invention are bound to lysines, drug loading may range from 1 to 80 drugs (D) per cell antibody, although an upper limit of 40, 20, 10 or 8 may be preferred. Compositions of ADC include collections of cell binding agents, e.g. antibodies, conjugated with a range of drugs, from 1 to 80, 1 to 40, 1 to 20, 1 to 10 or 1 to 8.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of drug ratio may also be determined. By ELISA, the averaged value of drug ratio in a particular preparation of ADC may be determined (Hamblett et al (2004) Clin. Cancer Res. 10:7063-7070; Sanderson et al (2005) Clin. Cancer Res. 11:843-852). However, the distribution of drug ratio values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, drug ratio may be limited by the number of attachment sites on the antibody. For example, an antibody may have only one or several cysteine thiol groups, or may have only one or several sufficiently reactive thiol groups through which a linker may be attached. Higher drug loading, e.g. drug ratio >5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the drug-linker intermediate (D-L) or linker reagent. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate (D-L) or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Doman et al (2009) Blood 114(13):2721-2729; U.S. Pat. No. 7,521,541; U.S. Pat. No. 7,723,485; WO2009/052249). The engineered cysteine thiols may react with linker reagents or the drug-linker reagents of the present invention which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the ADC compositions of the invention include mixtures of ADC where the antibody has one or more PBD drug moieties and where the drug moieties may be attached to the antibody at various amino acid residues.

In one embodiment, the average number of dimer PBD groups per cell binding agent is in the range 1 to 20. In some embodiments the range is selected from 1 to 12, 1 to 8, 2 to 8, 2 to 6, 2 to 4, and 4 to 8.

In some embodiments, there is two dimer pyrrolobenzodiazepine groups per cell binding agent.

In some embodiments, there is three dimer pyrrolobenzodiazepine groups per cell binding agent.

In some embodiments, there is four dimer pyrrolobenzodiazepine groups per cell binding agent.

Finally, the invention relates to an ADC as above described for use in the treatment of cancer.

Cancers can be preferably selected through Axl-related cancers including tumoral cells expressing or over-expressing whole or part of the protein Axl at their surface.

More particularly, said cancers are breast cancer, colon cancer, esophageal carcinoma, hepatocellular cancer, gastric cancer, glioma, lung cancer, melanoma, osteosarcoma, ovarian cancer, prostate cancer, rhabdomyo sarcoma, renal cancer, thyroid cancer, uterine endometrial cancer, schwannoma, neuroblastoma, oral squamous cancer, mesothelioma, leiomyosarcoma and any drug resistance phenomena or cancers. Another object of the invention is a pharmaceutical composition comprising the immunoconjugate as described in the specification.

For the avoidance of doubt, by drug resistance Axl-expressing cancers, it must be understood not only resistant cancers which initially express Axl but also cancers which initially do not express or overexpress Axl but which express Axl once they have become resistant to a previous treatment.

More particularly, the invention relates to a pharmaceutical composition comprising the ADC of the invention with at least an excipient and/or a pharmaceutical acceptable vehicle.

In the present description, the expression “pharmaceutically acceptable vehicle” or “excipient” is intended to indicate a compound or a combination of compounds entering into a pharmaceutical composition not provoking secondary reactions and which allows, for example, facilitation of the administration of the active compound(s), an increase in its lifespan and/or in its efficacy in the body, an increase in its solubility in solution or else an improvement in its conservation. These pharmaceutically acceptable vehicles and excipients are well known and will be adapted by the person skilled in the art as a function of the nature and of the mode of administration of the active compound(s) chosen.

Preferably, these ADCs will be administered by the systemic route, in particular by the intravenous route, by the intramuscular, intradermal, intraperitoneal or subcutaneous route, or by the oral route. In a more preferred manner, the composition comprising the ADCs according to the invention will be administered several times, in a sequential manner.

Their modes of administration, dosages and optimum pharmaceutical forms can be determined according to the criteria generally taken into account in the establishment of a treatment adapted to a patient such as, for example, the age or the body weight of the patient, the seriousness of his/her general condition, the tolerance to the treatment and the secondary effects noted.

Other characteristics and advantages of the invention appear in the continuation of the description with the examples and the figures whose legends are represented below.

### FIGURE LEGENDS

FIGS. 1A, 1B and 1C: Binding specificity of 1613F12 on the immobilized rhAxl-Fc protein (1A), rhDtk-Fc (1B) or rhMer-Fc (1C) proteins by ELISA.

FIG. 2: FACS analysis of the 1613F12 binding on human tumor cells

FIG. 3: ELISA experiments studying binding on rhAxl-Fc protein of both m1613F12 and hz1613F12.

FIGS. 4A, 4B and 4C: Immunofluorescence microscopy of SN12C cells after incubation with 1613F12 FIG. 4A—Photographs of the mIgG1 isotype control conditions both for the membrane and the intracellular staining FIG. 4B—Membrane staining FIG. 4C—Intracellular staining of both Axl receptor using 1613F12 and of the early endosome marker EEA1. Image overlays are presented bellow and co-localizations visualized are indicated by the arrows.

FIG. 5: Binding of hz1613F12 and hz1613F12-24 DAR4 and DAR2 to SN12C human renal tumor cells as determined by FACS analysis. Data represent the mean intensity of fluorescence obtained over a range dose of antibody or ADC.

FIG. 6: Binding of hz1613F12 and of hz1613F12-24 DAR4 and hz1613F12-24 DAR2 on rhAxl-Fc immobilized protein as determined by ELISA. Data represent the optical densities obtained over a range dose of the tested antibodies. Data were analysed using Prism application.

FIG. 7: Binding of hz1613F12 and hz1613F12-33 DAR4 to SN12C human renal tumor cells as determined by FACS analysis. Data represent the mean intensity of fluorescence obtained over a range dose of antibody or ADC.

FIG. 8: Binding of hz1613F12 and of hz1613F12-33 DAR4 on rhAxl-Fc immobilized protein as determined by ELISA. Data represent the optical densities obtained over a range dose of the tested antibodies. Data were analysed using Prism application.

FIG. 9: Concentration response cytotoxicity curves for hz1613F12-24 in a large variety of human tumor cells.

FIGS. 10A and 10B: Concentration response cytotoxicity curves for hz1613F12-24 in Axl+SN12C () and in the control Axl⁻ MCF7 ( [symbol] ) cell lines. A—at Day 3, B—at Day 6. Values of the EC₅₀ concentration was determined using Prism application with the regression analysis for each curve.

FIG. 11: hz1613F12-33 induces cell cytotoxicity of human Axl-expressing tumor cell lines. Percentages of cytotoxicity determined on SN12C, MDA-MB231 and MCF7 after a 6-day incubation period with hz1613F12-33.

FIG. 12: In vivo efficacy of the hz1613F12 (VH3/VL3)-24 and of the isotype control ADC c-9G4-24 injected i.p. at a dose of 0.9 mg/kg Q4d4 in SN12C grafted mice.

FIG. 13: In vivo efficacy of the hz1613F12 (VH1W55RN66K/VL3)-24 DAR2 injected i.p. at the dose 0.9 mg/kg Q7d4 starting at D20 after engraftment, compared to the PBS, in SN12C xenograft.

FIGS. 14A-14B: In vivo efficacy of the hz1613F12 (VH2.1W55RN66K/VL1I2V)-24 injected i.p. in SN12C xenograft compared to PBS and/or c9G4-24 ADC. A—At the dose of 1 mg/kg Q4d4. B—At the dose of 0.9 mg/kg Q7d4.

FIGS. 15A-15B-15C: In vivo efficacy of the hz1613F12 (VH3/VL3)-24 injected i.p. in a single dose of 5 mg/kg. A—NCI-H1299, B—Panc1 and C—MDA-MB-231.

FIG. 16: Survival analysis. hz1613F12-24 DAR2 antitumor activity against human A549 lung tumor cells implanted intrapleuraly (i.pl.) in nude mice. Hz1613F12-24 DAR2 ADC was administrated i.p. at the dose of 7 mg/kg and the capped-24 compound at a dose equivalent to 7 mg/kg ADC. Survival curves corresponding to the three groups of animals (hz1613F12-24, capped-24 and PBS) are presented. Statistical values obtained by applying a log-rank test as well as the T/C percentage are given.

### EXAMPLES

In the following examples, isotype control antibody used consists of a murine IgG1 referred as 9G4. It means that, in the following examples, the expressions mIgG1 control and 9G4 are similar.

### Example 1

### Generation of 1613F12

To generate murine monoclonal antibodies (Mabs) against human extracellular domain (ECD) of the Axl receptor, 5 BALB/c mice were immunized 5-times s.c. with 15-20·10⁶ CHO-Axl cells and twice with 20 μg of the rh Axl ECD. The first immunization was performed in presence of Complete Freund Adjuvant (Sigma, St Louis, Md., USA). Incomplete Freund adjuvant (Sigma) was added for following immunizations.

Three days prior to the fusion, immunized mice were boosted with both 20·10⁶ CHO-Axl cells and 20 μg of the rhAxl ECD with IFA.

To generate hybridomas, splenocytes and lymphocytes were prepared by perfusion of the spleen and by mincing of the proximal lymph nodes, respectively, harvested from 1 out of the 5 immunized mice (selected after sera titration) and fused to SP2/0-Ag14 myeloma cells (ATCC, Rockville, Md., USA). The fusion protocol is described by Kohler and Milstein (Nature, 256:495-497, 1975). Fused cells are then subjected to HAT selection. In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988).

Approximately 10 days after the fusion, colonies of hybrid cells were screened. For the primary screen, supernatants of hybridomas were evaluated for the secretion of Mabs raised against the Axl ECD protein using an ELISA. In parallel, a FACS analysis was performed to select Mabs able to bind to the cellular form of Axl present on the cell surface using both wt CHO and Axl expressing CHO cells.

As soon as possible, selected hybridomas were cloned by limit dilution and subsequently screened for their reactivity against the Axl ECD protein. Cloned Mabs were then isotyped using an Isotyping kit (cat #5300.05, Southern Biotech, Birmingham, Ala., USA). One clone obtained from each hybridoma was selected and expanded.

ELISA assays are performed as followed either using pure hybridoma supernatant or, when IgG content in supernatants was determined, titration was realized starting at 5 μg/ml. Then a ½ serial dilution was performed in the following 11 rows. Briefly, 96-well ELISA plates (Costar 3690, Corning, N.Y., USA) were coated 50 μl/well of the rh Axl-Fc protein (R and D Systems, cat N° 154-AL) or rhAxl ECD at 2 μg/ml in PBS overnight at 4° C. The plates were then blocked with PBS containing 0.5% gelatin (#22151, Serva Electrophoresis GmbH, Heidelberg, Germany) for 2 h at 37° C. Once the saturation buffer discarded by flicking plates, 50 μl of pure hybridoma cell supernatants or 50 μl of a 5 μg/ml solution were added to the ELISA plates and incubated for 1 h at 37° C. After three washes, 50 μl horseradish peroxidase-conjugated polyclonal goat anti-mouse IgG (#115-035-164, Jackson Immuno-Research Laboratories, Inc., West Grove, Pa., USA) was added at a 1/5000 dilution in PBS containing 0.1% gelatin and 0.05% Tween 20 (w:w) for 1 h at 37° C. Then, ELISA plates were washed 3-times and the TMB (#UP664782, Uptima, Interchim, France) substrate was added. After a 10 min incubation time at room temperature, the reaction was stopped using 1 M sulfuric acid and the optical density at 450 nm was measured.

For the selection by flow cytometry, 10⁵ cells (CHO wt or CHO-Axl) were plated in each well of a 96 well-plate in PBS containing 1% BSA and 0.01% sodium azide (FACS buffer) at 4° C. After a 2 min centrifugation at 2000 rpm, the buffer was removed and hybridoma supernatants or purified Mabs (1 μg/ml) to be tested were added. After 20 min of incubation at 4° C., cells were washed twice and an Alexa 488-conjugated goat anti-mouse antibody 1/500° diluted in FACS buffer (#A11017, Molecular Probes Inc., Eugene, USA) was added and incubated for 20 min at 4° C. After a final wash with FACS buffer, cells were analyzed by FACS (Facscalibur, Becton-Dickinson) after addition of propidium iodide to each tube at a final concentration of 40 μg/ml. Wells containing cells alone and cells incubated with the secondary Alexa 488-conjugated antibody were included as negative controls. Isotype controls were used in each experiment (Sigma, ref M90351MG). At least 5000 cells were assessed to calculate the mean value of fluorescence intensity (MFI).

The hybridoma producing the 1613F12 was selected as a candidate.

### Example 2

### Humanization of 1613F12

The use of mouse antibodies (Mabs) for therapeutic applications in humans generally results in a major adverse effect, patients raise a human anti-mouse antibody (HAMA) response, thereby reducing the efficacy of the treatment and preventing continued administration. One approach to overcome this problem is to humanize mouse Mabs by replacing mouse sequences by their human counterpart but without modifying the antigen binding activity. This can be achieved in two major ways: (i) by construction of mouse/human chimeric antibodies where the mouse variable regions are joined to human constant regions (Boulianne et al., 1984) and (ii) by grafting the complementarity determining regions (CDRs) from the mouse variable regions into carefully selected human variable regions and then joining these “re-shaped human” variable regions to human constant regions (Riechmann et al., 1988).

2.1 Humanization of the Light Chain Variable Domain VL

As a preliminary step, the nucleotide sequence of 1613F12 VL was compared to the murine germline gene sequences part of the IMGT database (http://www.imgt.org). Murine IGKV16-104*01 and IGKJ5*01 germline genes were identified. In order to identify the best human candidate for the CDR grafting, the human germline gene displaying the best identity with 1613F12 VL murine sequence has been searched. With the help of the IMGT database analyses tools, a possible acceptor human V regions for the murine 1613F12 VL CDRs was identified: IGKV1-27*01 and IGKJ4*02. In order to perform the humanization to the light chain variable domain each residue which is different between the human and mouse sequences was given a priority rank order. These priorities (1-4) were used to create 11 different humanized variants of the light chain variable region with up to 14 backmutations.

| | FR1-IMGT | CDR1-IMGT | FR2-IMGT | CD |
|---|---|---|---|---|
| 1613F12VL | DVQITQSPSYLATSPGETITINCRAS | KSI......SKY | LAWYQEKPGKTNKLLIY | SG |
| Homsap IGKV1-27*01 | DIQMTQSPSSLSASVGDRVTITCRAS | QGI......SNY | LAWYQQKPGKVPKLLIY | AA |
| | V I Y AT P ETI N | | E TN | |
| Priority | 1 1 3 34 4 433 2 | | 3 33 | |
| hz1613F12 (VL1) | DIQMTQSPSSLSASVGDRVTITCRAS | KSI......SKY | LAWYQQKPGKVPKLLIY | SG |
| hz1613F12 (VL1I2V) | DVQMTQSPSSLSASVGDRVTITCRAS | KSI......SKY | LAWYQQKPGKVPKLLIY | SG |
| hz1613F12 (VL1M4I) | DIQITQSPSSLSASVGDRVTITCRAS | KSI......SKY | LAWYQQKPGKVPKLLIY | SG |
| hz1613F12 (VL2.1) | DVQITQSPSSLSASVGDRVTITCRAS | KSI......SKY | LAWYQQKPGKVPKLLIY | SG |
| hz1613F12 (VL2.1V49T) | DVQITQSPSSLSASVGDRVTITCRAS | KSI......SKY | LAWYQQKPGKTPKLLIY | SG |
| hz1613F12 (VL2.1P50N) | DVQITQSPSSLSASVGDRVTITCRAS | KSI......SKY | LAWYQQKPGKVNKLLIY | SG |
| hz1613F12 (VL2.2) | DVQITQSPSSLSASVGDRVTINCRAS | KSI......SKY | LAWYQQKPGKVPKLLIY | SG |
| hz1613F12 (VL2.2V49T) | DVQITQSPSSLSASVGDRVTINCRAS | KSI......SKY | LAWYQQKPGKTPKLLIY | SG |
| hz1613F12 (VL2.2P50N) | DVQITQSPSSLSASVGDRVTINCRAS | KSI......SKY | LAWYQQKPGKVNKLLIY | SG |
| hz1613F12 (VL2.3) | DVQITQSPSSLSASVGDRVTINCRAS | KSI......SKY | LAWYQEKPGKTNKLLIY | SG |
| hz1613F12 (VL3) | DVQITQSPSYLAASVGDTITINCRAS | KSI......SKY | LAWYQEKPGKTNKLLIY | SG |

| | R2-IMGT | FR3-IMGT |
|---|---|---|
| 1613F12VL | .......S | TLQSGVP.SRFSGSG..SGTDFTLTISSLEPEDFAMYFC |
| Homsap IGKV1-27*01 | .......S | TLQSGVP.SRFSGSG..SGTDFTLTISSLQPEDVATYYC |
| | | E F M F |
| Priority | | 4 4 4 2 |
| hz1613F12 (VL1) | .......S | TLQSGVP.SRFSGSG..SGTDFTLTISSLQPEDVATYYC |
| hz1613F12 (VL1I2V) | .......S | TLQSGVP.SRFSGSG..SGTDFTLTISSLQPEDVATYYC |
| hz1613F12 (VL1M4I) | .......S | TLQSGVP.SRFSGSG..SGTDFTLTISSLQPEDVATYYC |
| hz1613F12 (VL2.1) | .......S | TLQSGVP.SRFSGSG..SGTDFTLTISSLQPEDVATYYC |
| hz1613F12 (VL2.1V49T) | .......S | TLQSGVP.SRFSGSG..SGTDFTLTISSLQPEDVATYYC |
| hz1613F12 (VL2.1P50N) | .......S | TLQSGVP.SRFSGSG..SGTDFTLTISSLQPEDVATYYC |
| hz1613F12 (VL2.2) | .......S | TLQSGVP.SRFSGSG..SGTDFTLTISSLQPEDVATYFC |
| hz1613F12 (VL2.2V49T) | .......S | TLQSGVP.SRFSGSG..SGTDFTLTISSLQPEDVATYFC |
| hz1613F12 (VL2.2P50N) | .......S | TLQSGVP.SRFSGSG..SGTDFTLTISSLQPEDVATYFC |
| hz1613F12 (VL2.3) | .......S | TLQSGVP.SRFSGSG..SGTDFTLTISSLQPEDVATYFC |
| hz1613F12 (VL3) | .......S | TLQSGVP.SRFSGSG..SGTDFTLTISSLQPEDVATYFC |
| | CDR3-IMGT | FR4-IMGT |
| 1613F12VL | QQHHEYPLT | FGAGTELELK |
| Homsap IGKJ4*02 | LT | FGGGTKVEIK |
| | | A EL L |
| Priority | | 3 33 4 |
| hz1613F12 (VL1) | QQHHEYPLT | FGGGTKVEIK |
| hz1613F12 (VL1I2V) | QQHHEYPLT | FGGGTKVEIK |
| hz1613F12 (VL1M4I) | QQHHEYPLT | FGGGTKVEIK |
| hz1613F12 (VL2.1) | QQHHEYPLT | FGGGTKVEIK |
| hz1613F12 (VL2.1V49T) | QQHHEYPLT | FGGGTKVEIK |
| hz1613F12 (VL2.1P50N) | QQHHEYPLT | FGGGTKVEIK |
| hz1613F12 (VL2.2) | QQHHEYPLT | FGGGTKVEIK |
| hz1613F12 (VL2.2V49T) | QQHHEYPLT | FGGGTKVEIK |
| hz1613F12 (VL2.2P50N) | QQHHEYPLT | FGGGTKVEIK |
| hz1613F12 (VL2.3) | QQHHEYPLT | FGGGTKVEIK |
| hz1613F12 (VL3) | QQHHEYPLT | FGAGTELEIK |

2.2 Humanization of the Heavy Chain Variable Domain VH

In order to identify the best human candidate for the CDR grafting, the mouse and human germline genes displaying the best identity with 1613F12 VH were searched. The nucleotide sequence of 1613F12 VH was aligned with both mouse and human germline gene sequences by using the sequence alignment software “IMGT/V-QUEST” which is part of the IMGT database. Alignments of amino acid sequences were also performed to verify the results of the nucleotide sequence alignment using the “Align X” software of the VectorNTI package. The alignment with mouse germline genes showed that the mouse germline V-gene IGHV14-3*02 and J-gene IGHJ2*01 are the most homologue mouse germline genes. Using the IMGT database the mouse D-gene germline IGHD1-1*01 was identified as homologous sequence. In order to select an appropriate human germline for the CDR grafting, the human germline gene with the highest homology to 1613F12 VH murine sequence was identified. With the help of IMGT databases and tools, the human IGHV1-2*02 germline gene and human IGHJ5*01 J germline gene were selected as human acceptor sequences for the murine 1613F12 VH CDRs. In order to perform the humanization to the heavy chain variable domain each residue which is different between the human and mouse sequences was given a priority rank order (1-4). These priorities were used to create 20 different humanized variants of the heavy chain variable region with up to 18 backmutations,

| | FR1-IMGT | CDR1-IMGT | FR2-IMGT | CD |
|---|---|---|---|---|
| | (1-26) | (27-38) | (39-55) | ( |
| 1613F12 | EVHLQQSGA.ELVKPGASVKLSCTAS | GFNI....RDTY | IHWVKQRPEQGLEWIGR | LD |
| Homsap IGHV1-2*02 | QVQLVQSGA.EVKKPGASVKVSCKAS | GYTF....TGYY | MHWVRQAPGQGLEWMGW | IN |
| | E H Q LV L T | | I K R E I R | |
| Priority | 3 2 3 33 3 3 | | 1 3 4 4 3 2 | |
| hz1613F12 (VH1) | QVQLVQSGA.EVKKPGASVKVSCKAS | GFNI....RDTY | MHWVRQAPGQGLEWMGW | LD |
| hz1613F12 (VH1M39I) | QVQLVQSGA.EVKKPGASVKVSCKAS | GFNI....RDTY | IHWVRQAPGQGLEWMGW | LD |
| hz1613F12 (VH1W55RN66K) | QVQLVQSGA.EVKKPGASVKVSCKAS | GFNI....RDTY | MHWVRQAPGQGLEWMGR | LD |
| hz1613F12 (VH1I84S) | QVQLVQSGA.EVKKPGASVKVSCKAS | GFNI....RDTY | MHWVRQAPGQGLEWMGW | LD |
| hz1613F12 (VH1S85N) | QVQLVQSGA.EVKKPGASVKVSCKAS | GFNI....RDTY | MHWVRQAPGQGLEWMGW | LD |
| hz1613F12 (VH1I84NS85N) | QVQLVQSGA.EVKKPGASVKVSCKAS | GFNI....RDTY | MHWVRQAPGQGLEWMGW | LD |
| hz1613F12 (VH2.1) | QVQLVQSGA.EVKKPGASVKVSCKAS | GFNI....RDTY | IHWVRQAPGQGLEWMGW | LD |
| hz1613F12 (VH2.1Q3H) | QVHLVQSGA.EVKKPGASVKVSCKAS | GFNI....RDTY | IHWVRQAPGQGLEWMGW | LD |
| hz1613F12 (VH2.1W55R) | QVQLVQSGA.EVKKPGASVKVSCKAS | GFNI....RDTY | IHWVRQAPGQGLEWMGR | LD |
| hz1613F12 (VH2.1N66K) | QVQLVQSGA.EVKKPGASVKVSCKAS | GFNI....RDTY | IHWVRQAPGQGLEWMGW | LD |
| hz1613F12 (VH2.1W55RN66K) | QVQLVQSGA.EVKKPGASVKVSCKAS | GFNI....RDTY | IHWVRQAPGQGLEWMGR | LD |
| hz1613F12 (VH2.1R805) | QVQLVQSGA.EVKKPGASVKVSCKAS | GFNI....RDTY | IHWVRQAPGQGLEWMGW | LD |
| hz1613F12 (VH2.1N66KR80S) | QVQLVQSGA.EVKKPGASVKVSCKAS | GFNI....RDTY | IHWVRQAPGQGLEWMGW | LD |
| hz1613F12 (VH2.2) | QVHLVQSGA.EVKKPGASVKVSCKAS | GFNI....RDTY | IHWVRQAPGQGLEWMGW | LD |
| hz1613F12 (VH2.2M89L) | QVHLVQSGA.EVKKPGASVKVSCKAS | GFNI....RDTY | IHWVRQAPGQGLEWMGW | LD |
| hz1613F12 (VH2.3) | QVQLQQSGA.EVKKPGASVKLSCTAS | GFNI....RDTY | IHWVRQAPGQGLEWMGW | LD |
| hz1613F12 (VH2.3W55R) | QVQLQQSGA.EVKKPGASVKLSCTAS | GFNI....RDTY | IHWVRQAPGQGLEWMGR | LD |
| hz1613F12 (VH2.3Q3HW55R) | QVHLQQSGA.EVKKPGASVKLSCTAS | GFNI....RDTY | IHWVRQAPGQGLEWMGR | LD |
| hz1613F12 (VH2.4) | QVQLQQSGA.EVKKPGASVKLSCTAS | GFNI....RDTY | IHWVRQAPGQGLEWIGR | LD |
| hz1613F12 (VH3) | EVHLQQSGA.ELVKPGASVKLSCTAS | GFNI....RDTY | IHWVKQAPGQGLEWIGR | LD |

| | R2-IMGT | FR3-IMGT |
|---|---|---|
| | 56-65) | (66-104) |
| 1613F12 | PA..NGHT | KYGPNFQ.GRATMTSDTSSNTAYLQLSSLTSEDTAVYYC |
| Homsap IGHV1-2*02 | PN..SGGT | NYAQKFQ.GRVTMTRDTSISTAYMELSRLRSDDTAVYYC |
| | | K GPN A S SN LQ S T E |
| Prority | | 2 344 4 2 11 33 4 4 4 |
| hz1613F12 (VH1) | PA..NGHT | NYAQKFQ.GRVTMTRDTSISTAYMELSRLRSDDTAVYYC |
| hz1613F12 (VH1M39I) | PA..NGHT | NYAQKFQ.GRVTMTRDTSISTAYMELSRLRSDDTAVYYC |
| hz1613F12 (VH1W55RN66K) | PA..NGHT | KYAQKFQ.GRVTMTRDTSISTAYMELSRLRSDDTAVYYC |
| hz1613F12 (VH1I84S) | PA..NGHT | NYAQKFQ.GRVTMTRDTSSSTAYMELSRLRSDDTAVYYC |
| hz1613F12 (VH1S85N) | PA..NGHT | NYAQKFQ.GRVTMTRDTSINTAYMELSRLRSDDTAVYYC |
| hz1613F12 (VH1I84NS85N) | PA..NGHT | NYAQKFQ.GRVTMTRDTSSNTAYMELSRLRSDDTAVYYC |
| hz1613F12 (VH2.1) | PA..NGHT | NYAQKFQ.GRVTMTRDTSSNTAYMELSRLRSDDTAVYYC |
| hz1613F12 (VH2.1Q3H) | PA..NGHT | NYAQKFQ.GRVTMTRDTSSNTAYMELSRLRSDDTAVYYC |
| hz1613F12 (VH2.1W55R) | PA..NGHT | NYAQKFQ.GRVTMTRDTSSNTAYMELSRLRSDDTAVYYC |
| hz1613F12 (VH2.1N66K) | PA..NGHT | KYAQKFQ.GRVTMTRDTSSNTAYMELSRLRSDDTAVYYC |
| hz1613F12 (VH2.1W55RN66K) | PA..NGHT | KYAQKFQ.GRVTMTRDTSSNTAYMELSRLRSDDTAVYYC |
| hz1613F12 (VH2.1R80S) | PA..NGHT | NYAQKFQ.GRVTMTSDTSSNTAYMELSRLRSDDTAVYYC |
| hz1613F12 (VH2.1N66KR80S) | PA..NGHT | KYAQKFQ.GRVTMTSDTSSNTAYMELSRLRSDDTAVYYC |
| hz1613F12 (VH2.2) | PA..NGHT | KYAQKFQ.GRVTMTSDTSSNTAYMELSRLRSDDTAVYYC |
| hz1613F12 (VH2.2M89L) | PA..NGHT | KYAQKFQ.GRVTMTSDTSSNTAYLELSRLRSDDTAVYYC |
| hz1613F12 (VH2.3) | PA..NGHT | KYAQKFQ.GRVTMTSDTSSNTAYMELSRLRSDDTAVYYC |
| hz1613F12 (VH2.3W55R) | PA..NGHT | KYAQKFQ.GRVTMTSDTSSNTAYMELSRLRSDDTAVYYC |
| hz1613F12 (VH2.3Q3HW55R) | PA..NGHT | KYAQKFQ.GRVTMTSDTSSNTAYMELSRLRSDDTAVYYC |
| hz1613F12 (VH2.4) | PA..NGHT | KYAQKFQ.GRVTMTSDTSSNTAYLELSRLRSDDTAVYYC |
| hz1613F12 (VH3) | PA..NGHT | KYGQKFQ.GRVIMISDISSNTAYLQLSRLRSDDTAVYYC |

| | CDR3-IMGT | FR4-IMGT |
|---|---|---|
| 1613F12VH | ARGAYYYGSSGLFYFDY | WGQGTLVTVSS |
| Homsap IGHJ5*01 | | WGQGTLVTVSS |
| | | TLS |
| Prority | | 444 |
| hz1613F12 (VH1) | ARGAYYYGSSGLFYFDY | WGQGTLVTVSS |
| hz1613F12 (VH1M39I) | ARGAYYYGSSGLFYFDY | WGQGTLVTVSS |
| hz1613F12 (VH1W55RN66K) | ARGAYYYGSSGLFYFDY | WGQGTLVTVSS |
| hz1613F12 (VH1I84S) | ARGAYYYGSSGLFYFDY | WGQGTLVTVSS |
| hz1613F12 (VH1S85N) | ARGAYYYGSSGLFYFDY | WGQGTLVTVSS |
| hz1613F12 (VH1I84NS85N) | ARGAYYYGSSGLFYFDY | WGQGTLVTVSS |
| hz1613F12 (VH2.1) | ARGAYYYGSSGLFYFDY | WGQGTLVTVSS |
| hz1613F12 (VH2.1Q3H) | ARGAYYYGSSGLFYFDY | WGQGTLVTVSS |
| hz1613F12 (VH2.1W55R) | ARGAYYYGSSGLFYFDY | WGQGTLVTVSS |
| hz1613F12 (VH2.1N66K) | ARGAYYYGSSGLFYFDY | WGQGTLVTVSS |
| hz1613F12 (VH2.1W55RN66K) | ARGAYYYGSSGLFYFDY | WGQGTLVTVSS |
| hz1613F12 (VH2.1R80S) | ARGAYYYGSSGLFYFDY | WGQGTLVTVSS |
| hz1613F12 (VH2.1N66KR80S) | ARGAYYYGSSGLFYFDY | WGQGTLVTVSS |
| hz1613F12 (VH2.2) | ARGAYYYGSSGLFYFDY | WGQGTLVTVSS |
| hz1613F12 (VH2.2 M89L) | ARGAYYYGSSGLFYFDY | WGQGTLVTVSS |
| hz1613F12 (VH2.3) | ARGAYYYGSSGLFYFDY | WGQGTLVTVSS |
| hz1613F12 (VH2.3W55R) | ARGAYYYGSSGLFYFDY | WGQGTLVTVSS |
| hz1613F12 (VH2.3Q3HW55R) | ARGAYYYGSSGLFYFDY | WGQGTLVTVSS |
| hz1613F12 (VH2.4) | ARGAYYYGSSGLFYFDY | WGQGTLVTVSS |
| hz1613F12 (VH3) | ARGAYYYGSSGLFYFDY | WGQGTLVTVSS |

### Example 3

### Axl Binding Specificity

In this example, the binding of 1613F12 was first studied on the rhAxl-Fc protein. Then, its binding on the two other members of the TAM family, rhDtk-Fc and rhMer-Fc, was studied.

Briefly, the recombinant human Axl-Fc (R and D systems, cat N° 154AL/CF), rhDtk (R and D Systems, cat N° 859-DK) or rhMer-Fc (R and D Systems, cat N° 891-MR) proteins were coated overnight at 4° C. to Immulon II 96-well plates and, after a 1 h blocking step with a 0.5% gelatine solution, 1613F12 was added for an additional 1 h at 37° C. at starting concentration of 5 μg/ml (3.33 10⁻⁸M). Then ½ serial dilutions were done over 12 columns. Plates were washed and a goat anti-mouse (Jackson) specific IgG-HRP was added for 1 h at 37° C. Reaction development was performed using the TMB substrate solution. The isotype control antibody mIgG1 and the commercial anti-Axl Mab 154 antibody were also used in parallel. Coating controls were performed in presence of a goat anti-human IgG Fc polyclonal serum labelled with HRP (Jackson, ref 109-035-098) and/or in presence of a HRP-coupled anti-Histidine antibody (R and D Systems, ref: MAB050H).

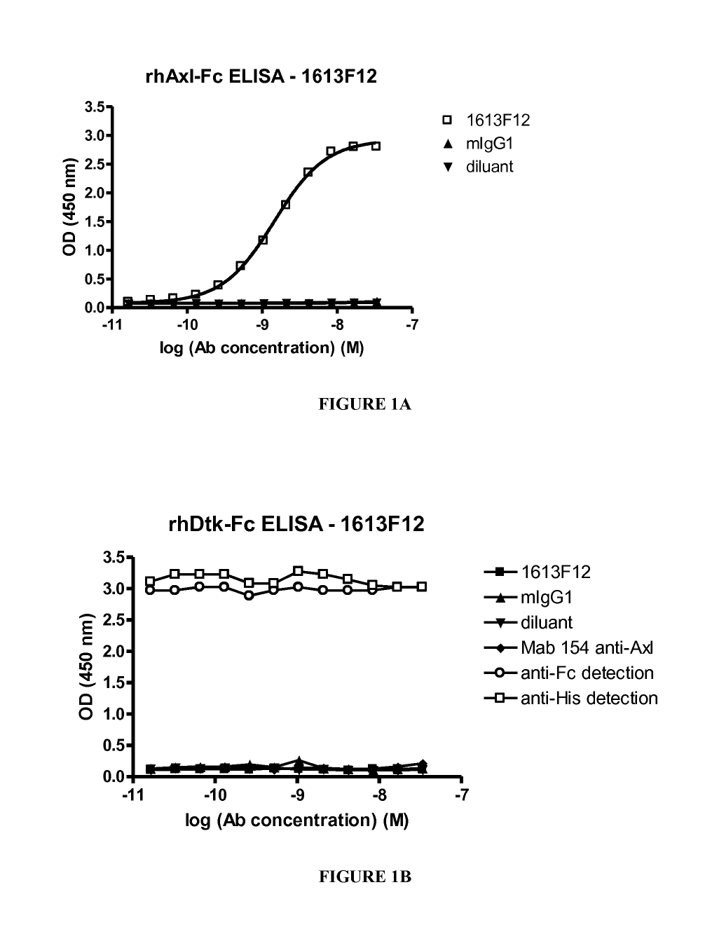

Results are represented in FIGS. 1A, 1B and 1C, respectively.

This example shows that 1613F12 only binds to the rhAxl-Fc protein and does not bind on the two other members of the TAM family, rhDtk or rhMer. No cross-specificity of binding of 1613F12 is observed between TAM members. No non specific binding was observed in absence of primary antibody (diluant). No binding was observed in presence of the isotype control antibody.

### Example 4

### 1613F12 Recognized the Cellular Form of Axl Expressed on Human Tumor Cells

Cell surface Axl expression level on human tumor cells was first established using a commercial Axl antibody (R and D Systems, ref: MAB154) in parallel of calibration beads to allow the quantification of Axl expression level. Secondly, binding of the cell-surface Axl was studied using 1613F12.

For cell surface binding studies, two fold serial dilutions of a 10 μg/ml (6.66 10⁻⁸ M) primary antibody solution (1613F12, MAB154 or mIgG1 isotype control 9G4 Mab) are prepared and are applied on 2·10⁵ cells for 20 min at 4° C. After 3 washes in phosphate-buffered saline (PBS) supplemented with 1% BSA and 0.01% NaN₃, cells were incubated with secondary antibody Goat anti-mouse Alexa 488 ( 1/500° dilution) for 20 minutes at 4° C. After 3 additional washes in PBS supplemented with 1% BSA and 0.1% NaN₃, cells were analyzed by FACS (Facscalibur, Becton-Dickinson). At least 5000 cells were assessed to calculate the mean value of fluorescence intensity.

For quantitative ABC determination using MAB154, QIFIKIT® calibration beads are used. Then, the cells are incubated, in parallel with the QIFIKIT® beads, with Polyclonal Goat Anti-Mouse Immunoglobulins/FITC, Goat F(ab′)₂, at saturating concentration. The number of antigenic sites on the specimen cells is then determined by interpolation of the calibration curve (the fluorescence intensity of the individual bead populations against the number of Mab molecules on the beads.

4.1. Quantification of Cell-Surface Axl Expression Level

Axl expression level on the surface of human tumor cells was determined by flow cytometry using indirect immuno fluorescence assay (QIFIKIT® method (Dako, Denmark), a quantitative flow cytometry kit for assessing cell surface antigens. A comparison of the mean fluorescence intensity (MFI) of the known antigen levels of the beads via a calibration graph permits determination of the antibody binding capacity (ABC) of the cell lines.

Table 4 presents Axl expression level detected on the surface of various human tumor cell lines (SN12C, Calu-1, MDA-MB435S, MDA-MB231, NCI-H125, MCF7, Panc1) as determined using QIFIKIT® using the MAB154 (R and D Systems). Values are given as Antigen binding complex (ABC).

TABLE 4

| | MCF7 | NCI-H125 | MDA-MB-435S | Panc1 | MDA-MB-231 | Calu-1 | SN12C |
|---|---|---|---|---|---|---|---|
| Tumor type/organ | Breast | NSCLC | Breast | Pancreas | Breast | Lung | Renal |
| ABC (Qifikit) | 71 | 5 540 | 17 814 | 36 809 | 61 186 | >100 000 | >100 000 |

Results obtained with MAB154 showed that Axl receptor is expressed at various levels depending of the considered human tumor cell.

4.2. Axl Detection by 1613F12 on Human Tumor Cells

More specifically, Axl binding was studied using 1613F12.

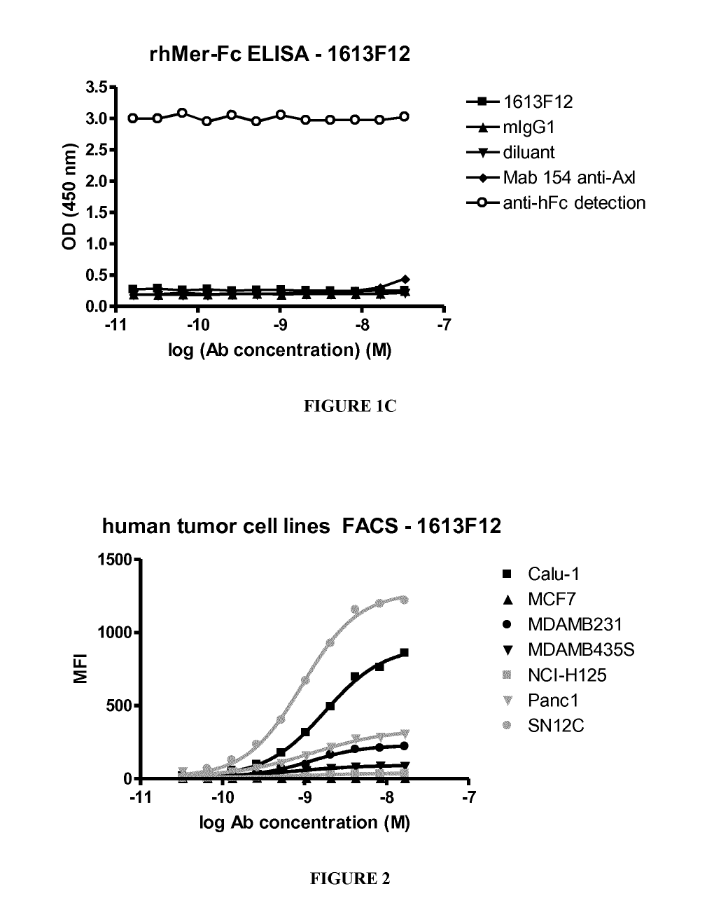

1613F12 dose response curves were prepared. MFIs obtained using the various human tumor cells were then analysed with Prism software. Data are presented in FIG. 2.

Data indicate that 1613F12 binds specifically to the membrane Axl receptor as attested by the saturation curve profiles. However different intensities of labelling were observed, revealing variable levels of cell-surface Axl receptor on human tumor cells. No binding of Axl receptor was observed using MCF7 human breast tumor cell line.

### Example 5

### Validation of hz1613F12 vs. m1613F12

In order to establish whether hz1613F12 was comparable to its murine form, binding experiments were performed by ELISA using rhAxl-Fc protein assays.

In this assay, 96 well plates (Immulon II, Thermo Fisher) were coated with a 5 μg/ml of 1613F12 solution in 1×PBS, overnight at 4° C. After a saturation step, a range of rh Axl-Fc protein (R and D Systems, ref: 154-AL) is incubated for 1 hour at 37° C. on the coated plates. For the revelation step, a biotinylated-Axl antibody (in house product) was added at 0.85 μg/ml for 1 hour at 37° C. This Axl antibody belongs to a distinct epitopic group. Then an avidin-horseradish peroxidase solution at 1/2000° in diluent buffer is added to the wells. Then the TMB substrate solution is added for 5 min. After addition of the peroxydase stop solution, the absorbance at 405 nm was measured with a microplate reader.

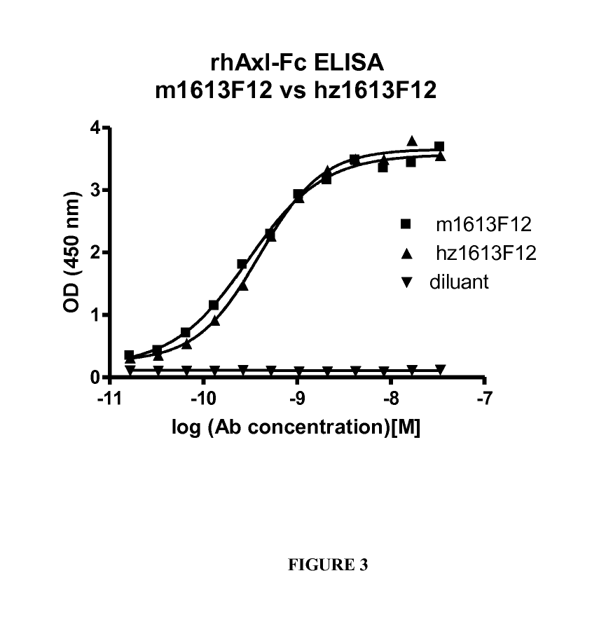

FIG. 3 shows that both murine and humanized versions of 1613F12 bind similarly the rhAxl-Fc protein.

### Example 6

### 1613F12 Internalization Study Using Fluorescent Immunocytochemistry Labelling

Complementary internalization results are obtained by confocal microscopy using indirect fluorescent labelling method.

Briefly, SN12C tumor cell line was cultured in RMPI1640 with 1% L-glutamine and 10% of FCS for 3 days before experiment. Cells were then detached using trypsin and plated in 6-multiwell plate containing coverslide in RPMI1640 with 1% L-glutamine and 5% FCS. The next day, 1613F12 was added at 10 μg/ml. Cells treated with an irrelevant antibody were also included. The cells were then incubated for 1 h and 2 h at 37° C., 5% CO₂. For T 0 h, cells were incubated for 30 minutes at 4° C. to determine antibody binding on cell surface. Cells were washed with PBS and fixed with paraformaldehyde for 15 minutes. Cells were rinsed and incubated with a goat anti-mouse IgG Alexa 488 antibody for 60 minutes at 4° C. to identify remaining antibody on the cell surface. To follow antibody penetration into the cells, cells were fixed and permeabilized with saponin. A goat anti-mouse IgG Alexa 488 (Invitrogen) was used to stained both the membrane and the intracellular antibody. Early endosomes were identified using a rabbit polyclonal antibody against EEA1 revealed with a goat anti-rabbit IgG-Alexa 555 antibody (Invitrogen). Cells were washed three times and nuclei were stained using Draq5. After staining, cells were mounted in Prolong Gold mounting medium (Invitrogen) and analyzed by using a Zeiss LSM 510 confocal microscope.

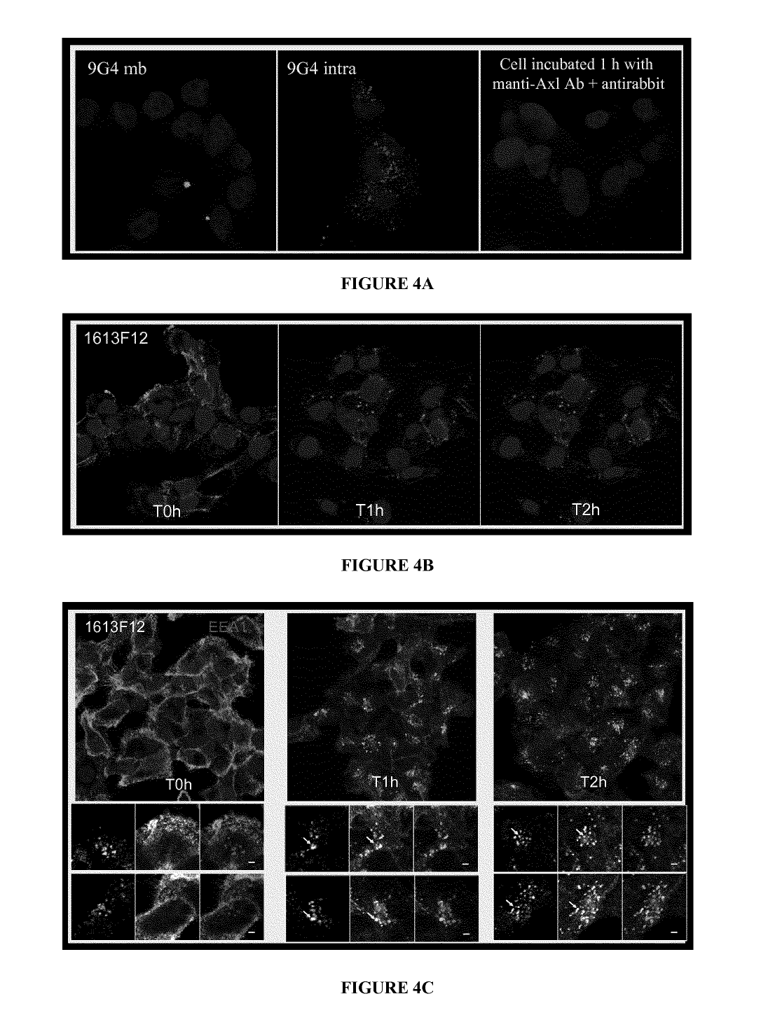

Photographs are presented in FIGS. 4A-4C.

Images were obtained by confocal microscopy. In presence of the mIgG1 isotype control (9G4), neither membrane staining nor intracellular labelling is observed (FIG. 4A). A progressive loss of the membrane anti-Axl labelling is observed as soon as after 1 h incubation of the SN12C cells with 1613F12 (FIG. 4B). Intracellular accumulation of 1613F12 antibody is clearly observed at 1 h and 2 h (FIG. 4C). Intracellular antibody co-localizes with EEA1, an early endosome marker. These photographs confirm the internalization of 1613F12 into SN12C cells.

### Example 7

### Synthesis of the PBD Dimers of the Invention

7.1 General Experimental Methods

Optical rotations were measured on an ADP 220 polarimeter (Bellingham Stanley Ltd.) and concentrations (c) are given in g/100 mL. Melting points were measured using a digital melting point apparatus (Electrothermal). IR spectra were recorded on a Perkin-Elmer Spectrum 1000 FT IR Spectrometer. ¹H and ¹³C NMR spectra were acquired at 300 K using a Bruker Avance NMR spectrometer at 400 and 100 MHz, respectively. Chemical shifts are reported relative to TMS (6=0.0 ppm), and signals are designated as s (singlet), d (doublet), t (triplet), dt (double triplet), dd (doublet of doublets), ddd (double doublet of doublets) or m (multiplet), with coupling constants given in Hertz (Hz). Mass spectroscopy (MS) data were collected using a Waters Micromass ZQ instrument coupled to a Waters 2695 HPLC with a Waters 2996 PDA. Waters Micromass ZQ parameters used were: Capillary (kV), 3.38; Cone (V), 35; Extractor (V), 3.0; Source temperature (° C.), 100; Desolvation Temperature (° C.), 200; Cone flow rate (L/h), 50; De-solvation flow rate (L/h), 250. High-resolution mass spectroscopy (HRMS) data were recorded on a Waters Micromass QTOF Global in positive W-mode using metal-coated borosilicate glass tips to introduce the samples into the instrument. Thin Layer Chromatography (TLC) was performed on silica gel aluminium plates (Merck 60, F₂₅₄), and flash chromatography utilised silica gel (Merck 60, 230-400 mesh ASTM). Except for the HOBt (NovaBiochem) and solid-supported reagents (Argonaut), all other chemicals and solvents were purchased from Sigma-Aldrich and were used as supplied without further purification. Anhydrous solvents were prepared by distillation under a dry nitrogen atmosphere in the presence of an appropriate drying agent, and were stored over 4 Å molecular sieves or sodium wire. Petroleum ether refers to the fraction boiling at 40-60° C.

General LC/MS conditions: The HPLC (Waters Alliance 2695) was run using a mobile phase of water (A) (formic acid 0.1%) and acetonitrile (B) (formic acid 0.1%). Gradient: initial composition 5% B over 1.0 min then 5% B to 95% B over 2.5 min. The composition was held for 0.5 min at 95% B, and then returned to 5% B in 0.1 minutes and held there for 0.9 min. Total gradient run time equals 5 min. Flow rate 3.0 mL/min, 400 μL was split via a zero dead volume tee piece which passes into the mass spectrometer. Wavelength detection range: 220 to 400 nm. Function type: diode array (535 scans). Column: Phenomenex® Onyx Monolithic C18 50×4.60 mm

7.2: Synthesis of Drug Moiety 24 (Referred Hereinafter as “24”)

### (i) (S)-(2-amino-5-methoxy-4-((triisopropylsilyl)oxy)phenyl)(2-(((tert-butyldimethylsilyl)oxy)methyl)-4-methyl-2,3-dihydro-1H-pyrrol-)methanone (9)

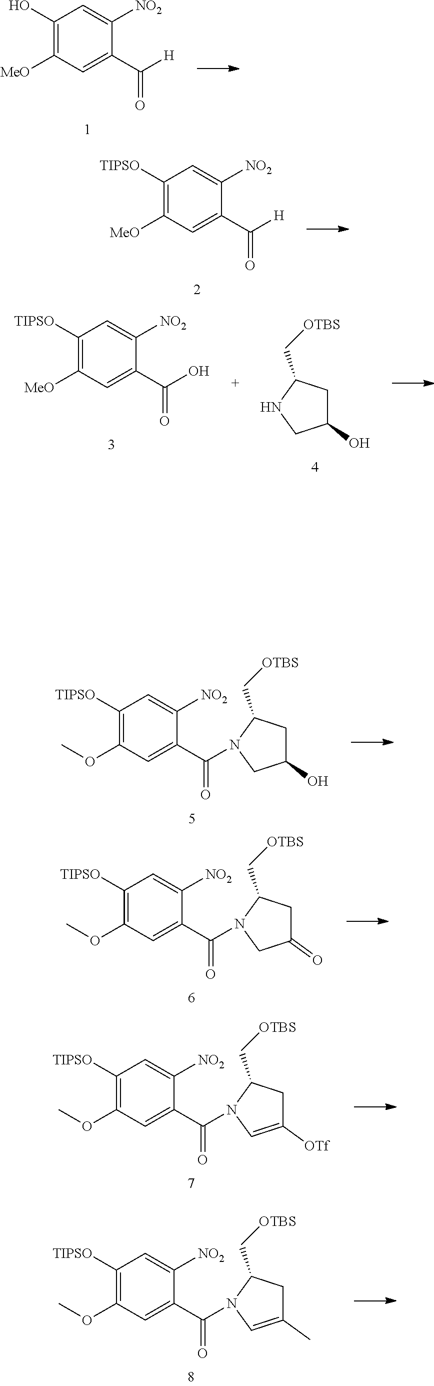

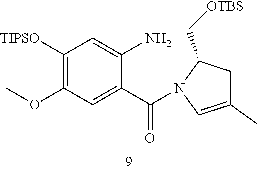

### (a) 5-methoxy-2-nitro-4-((triisopropylsilyl)oxy)benzaldehyde (2)

Neat triisopropylsilylchloride (56.4 mL, 262 mmol) was added to a mixture of imidazole (48.7 g, 715.23 mmol) and 4-hydroxy-5-methoxy-2-nitrobenzaldehyde 1 (47 g, 238 mmol) (ground together). The mixture was heated until the phenol and imidazole melted and went into solution (100° C.). The reaction mixture was allowed to stir for 15 minutes and was then allowed to cool, whereupon a solid was observed to form at the bottom of the flask (imidazole chloride). The reaction mixture was diluted with 5% EtOAc/hexanes and loaded directly onto silica gel and the pad was eluted with 5% EtOAc/hexanes, followed by 10% EtOAc/hexanes (due to the low excess, very little unreacted TIPSC1 was found in the product). The desired product was eluted with 5% ethyl acetate in hexane. Excess eluent was removed by rotary evaporation under reduced pressure, followed by drying under high vacuum to afford a crystalline light sensitive solid (74.4 g, 88%). Purity satisfactory by LC/MS (4.22 min (ES+) m/z (relative intensity) 353.88 ([M+H]^{+.}, 100)); ¹H NMR (400 MHz, CDCl₃) δ 10.43 (s, 1H), 7.60 (s, 1H), 7.40 (s, 1H), 3.96 (s, 3H), 1.35-1.24 (m, 3H), 1.10 (m, 18H).

### (b) 5-methoxy-2-nitro-4-((triisopropylsilyl)oxy)benzoic acid (3)

A solution of sodium chlorite (47.3 g, 523 mmol, 80% technical grade) and sodium dihydrogenphosphate monobasic (35.2 g, 293 mmol) (NaH₂PO₄) in water (800 mL) was added to a solution of compound 2 (74 g, 209 mmol) in tetrahydrofuran (500 mL) at room temperature. Hydrogen peroxide (60% w/w, 140 mL, 2.93 mol) was immediately added to the vigorously stirred biphasic mixture. The reaction mixture evolved gas (oxygen), the starting material dissolved and the temperature of the reaction mixture rose to 45° C. After 30 minutes LC/MS revealed that the reaction was complete. The reaction mixture was cooled in an ice bath and hydrochloric acid (1 M) was added to lower the pH to 3 (this step was found unnecessary in many instances, as the pH at the end of the reaction is already acidic; please check the pH before extraction). The reaction mixture was then extracted with ethyl acetate (1 L) and the organic phases washed with brine (2×100 mL) and dried over magnesium sulphate. The organic phase was filtered and excess solvent removed by rotary evaporation under reduced pressure to afford the product 6 in quantitative yield as a yellow solid. LC/MS (3.93 min (ES−) m/z (relative intensity) 367.74 ([M−H]⁻; 100)); ¹H NMR (400 MHz, CDCl₃) δ 7.36 (s, 1H), 7.24 (s, 1H), 3.93 (s, 3H), 1.34-1.22 (m, 3H), 1.10 (m, 18H).

### (c) ((2S,4R)-2-(((tert-butyldimethylsilyl)oxy)methyl)-4-hydroxypyrrolidin-1-yl)(5-methoxy-2-nitro-4-((triisopropylsilyl)oxy)phenyl)methanone (5)

DCC (29.2 g, 141 mmol, 1.2 eq) was added to a solution of acid 3 (43.5 g, 117.8 mmol, leg), and hydroxybenzotriazole hydrate (19.8 g, 129.6 mmol, 1.1 eq) in dichloromethane (200 mL) at 0° C. The cold bath was removed and the reaction was allowed to proceed for 30 mins at room temperature, at which time a solution of (2S,4R)-2-t-butyldimethylsilyloxymethyl-4-hydroxypyrrolidine 4 (30 g, 129.6 mmol, 1.1 eq) and triethylamine (24.66 mL, 176 mmol, 1.5 eq) in dichloromethane (100 mL) was added rapidly at −10° C. under argon (on large scale, the addition time could be shortened by cooling the reaction mixture even further. The reaction mixture was allowed to stir at room temperature for 40 minutes to 1 hour and monitored by LC/MS and TLC (EtOAc). The solids were removed by filtration over celite and the organic phase was washed with cold aqueous 0.1 M HCl until the pH was measured at 4 or 5. The organic phase was then washed with water, followed by saturated aqueous sodium bicarbonate and brine. The organic layer was dried over magnesium sulphate, filtered and excess solvent removed by rotary evaporation under reduced pressure. The residue was subjected to column flash chromatography (silica gel; gradient 40/60 ethyl acetate/hexane to 80/20 ethyl acetate/hexane). Excess solvent was removed by rotary evaporation under reduced pressure afforded the pure product 13, (45.5 g of pure product 66%, and 17 g of slightly impure product, 90% in total). LC/MS 4.43 min (ES+) m/z (relative intensity) 582.92 ([M+H]^{+.}, 100); ¹H NMR (400 MHz, CDCl₃) δ 7.66 (s, 1H), 6.74 (s, 1H), 4.54 (s, 1H), 4.40 (s, 1H), 4.13 (s, 1H), 3.86 (s, 3H), 3.77 (d, J=9.2 Hz, 1H), 3.36 (dd, J=11.3, 4.5 Hz, 1H), 3.14-3.02 (m, 1H), 2.38-2.28 (m, 1H), 2.10 (ddd, J=13.3, 8.4, 2.2 Hz, 1H), 1.36-1.19 (m, 3H), 1.15-1.05 (m, 18H), 0.91 (s, 9H), 0.17-0.05 (m, 6H), (presence of rotamers).

### (d) (S)-5-(((tert-butyldimethylsilyl)oxy)methyl)-1-(5-methoxy-2-nitro-4-((triisopropylsilyl)oxy)benzoyl)pyrrolidin-3-one (6)

TCCA (8.82 g, 40 mmol, 0.7 eq) was added to a stirred solution of 5 (31.7 g, 54 mmol, 1 eq) and TEMPO (0.85 g, 5.4 mmol, 0.1 eq) in dry dichloromethane (250 mL) at 0° C. The reaction mixture was vigorously stirred for 20 minutes, at which point TLC (50/50 ethyl acetate/hexane) revealed complete consumption of the starting material. The reaction mixture was filtered through celite and the filtrate washed with aqueous saturated sodium bicarbonate (100 mL), sodium thiosulphate (9 g in 300 mL), brine (100 mL) and dried over magnesium sulphate. Rotary evaporation under reduced pressure afforded product 6 in quantitative yield. LC/MS 4.52 min (ES+) m/z (relative intensity) 581.08 ([M+H]^{+.}, 100);

¹H NMR (400 MHz, CDCl₃) δ 7.78-7.60 (m, 1H), 6.85-6.62 (m, 1H), 4.94 (dd, J=30.8, 7.8 Hz, 1H), 4.50-4.16 (m, 1H), 3.99-3.82 (m, 3H), 3.80-3.34 (m, 3H), 2.92-2.17 (m, 2H), 1.40-1.18 (m, 3H), 1.11 (t, J=6.2 Hz, 18H), 0.97-0.75 (m, 9H), 0.15-−0.06 (m, 6H), (presence of rotamers).

### (e) (S)-5-(((tert-butyldimethylsilyl)oxy)methyl)-1-(5-methoxy-2-nitro-4-((triisopropylsilyl)oxy)benzoyl)-4,5-dihydro-1H-pyrrol-3-yl trifluoromethanesulfonate (7)

Triflic anhydride (27.7 mL, 46.4 g, 165 mmol, 3 eq) was injected (temperature controlled) to a vigorously stirred suspension of ketone 6 (31.9 g, 55 mmol, 1 eq) in dry dichloromethane (900 mL) in the presence of 2,6-lutidine (25.6 mL, 23.5 g, 220 mmol, 4 eq, dried over sieves) at −50° C. (acetone/dry ice bath). The reaction mixture was allowed to stir for 1.5 hours when LC/MS, following a mini work-up (water/dichloromethane), revealed the reaction to be complete. Water was added to the still cold reaction mixture and the organic layer was separated and washed with saturated sodium bicarbonate, brine and magnesium sulphate. The organic phase was filtered and excess solvent was removed by rotary evaporation under reduced pressure. The residue was subjected to column flash chromatography (silica gel; 10/90 v/v ethyl acetate/hexane), removal of excess eluent afforded the product 7 (37.6 g, 96%) LC/MS, method 2, 4.32 min (ES+) m/z (relative intensity) 712.89 ([M+H]^{+.}, 100); ¹H NMR (400 MHz, CDCl₃) δ 7.71 (s, 1H), 6.75 (s, 1H), 6.05 (d, J=1.8 Hz, 1H), 4.78 (dd, J=9.8, 5.5 Hz, 1H), 4.15-3.75 (m, 5H), 3.17 (ddd, J=16.2, 10.4, 2.3 Hz, 1H), 2.99 (ddd, J=16.3, 4.0, 1.6 Hz, 1H), 1.45-1.19 (m, 3H), 1.15-1.08 (m, 18H), 1.05 (s, 6H), 0.95-0.87 (m, 9H), 0.15-0.08 (m, 6H).

### (f) (S)-(2-(((tert-butyldimethylsilyl)oxy)methyl)-4-methyl-2,3-dihydro-1H-pyrrol-1-yl)(5-methoxy-2-nitro-4-((triisopropylsilyl)oxy)phenyl)methanone (8)

Triphenylarsine (1.71 g, 5.60 mmol, 0.4 eq) was added to a mixture of triflate 7 (10.00 g, 14 mmol, 1 eq), methylboronic acid (2.94 g, 49.1 mmol, 3.5 eq), silver oxide (13 g, 56 mmol, 4 eq) and potassium phosphate tribasic (17.8 g, 84 mmol, 6 eq) in dry dioxane (80 mL) under an argon atmosphere. The reaction was flushed with argon 3 times and bis(benzonitrile)palladium(II) chloride (540 mg, 1.40 mmol, 0.1 eq) was added. The reaction was flushed with argon 3 more times before being warmed instantaneously to 110° C. (the drysyn heating block was previously warmed to 110° C. prior addition of the flask). After 10 mins the reaction was cooled to room temperature and filtered through a pad celite. The solvent was removed by rotary evaporation under reduced pressure. The resulting residue was subjected to column flash chromatography (silica gel; 10% ethyl acetate/hexane). Pure fractions were collected and combined, and excess eluent was removed by rotary evaporation under reduced pressure afforded the product 8 (4.5 g, 55%). LC/MS, 4.27 min (ES+) m/z (relative intensity) 579.18 ([M+H]^{+.}, 100); ¹H NMR (400 MHz, CDCl₃) δ 7.70 (s, 1H), 6.77 (s, 1H), 5.51 (d, J=1.7 Hz, 1H), 4.77-4.59 (m, 1H), 3.89 (s, 3H), 2.92-2.65 (m, 1H), 2.55 (d, J=14.8 Hz, 1H), 1.62 (d, J=1.1 Hz, 3H), 1.40-1.18 (m, 3H), 1.11 (s, 9H), 1.10 (s, 9H), 0.90 (s, 9H), 0.11 (d, J=2.3 Hz, 6H).

### (g) (S)-(2-amino-5-methoxy-4-((triisopropylsilyl)oxy)phenyl)(2-(((tert-butyldimethylsilyl)oxy)methyl)-4-methyl-2,3-dihydro-1H-pyrrol-1-yl)methanone (9)

Zinc powder (28 g, 430 mmol, 37 eq) was added to a solution of compound 8 (6.7 g, 11.58 mmol) in 5% formic acid in ethanol v/v (70 mL) at around 15° C. The resulting exotherm was controlled using an ice bath to maintain the temperature of the reaction mixture below 30° C. After 30 minutes the reaction mixture was filtered through a pad of celite. The filtrate was diluted with ethyl acetate and the organic phase was washed with water, saturated aqueous sodium bicarbonate and brine. The organic phase was dried over magnesium sulphate, filtered and excess solvent removed by rotary evaporation under reduced pressure. The resulting residue was subjected to flash column chromatography (silica gel; 10% ethyl acetate in hexane). The pure fractions were collected and combined and excess solvent was removed by rotary evaporation under reduced pressure to afford the product 9 (5.1 g, 80%). LC/MS, 4.23 min (ES+) m/z (relative intensity) 550.21 ([M+H]^{+.}, 100); ¹H NMR (400 MHz, CDCl₃) δ 7.28 (s, 1H), 6.67 (s, 1H), 6.19 (s, 1H), 4.64-4.53 (m, J=4.1 Hz, 1H), 4.17 (s, 1H), 3.87 (s, 1H), 3.77-3.69 (m, 1H), 3.66 (s, 3H), 2.71-2.60 (m, 1H), 2.53-2.43 (m, 1H), 2.04-1.97 (m, J=11.9 Hz, 1H), 1.62 (s, 3H), 1.26-1.13 (m, 3H), 1.08-0.99 (m, 18H), 0.82 (s, 9H), 0.03-−0.03 (m, J=6.2 Hz, 6H).

### (ii) (11S,11aS)-allyl 11-((tert-butyldimethylsilyl)oxy)-8-((5-iodopentyl)oxy)-7-methoxy-2-methyl-5-oxo-11,11a-dihydro-1H-benzo[e]pyrrolo[1,2-a][1,4]diazepine-10(5H)-carboxylate

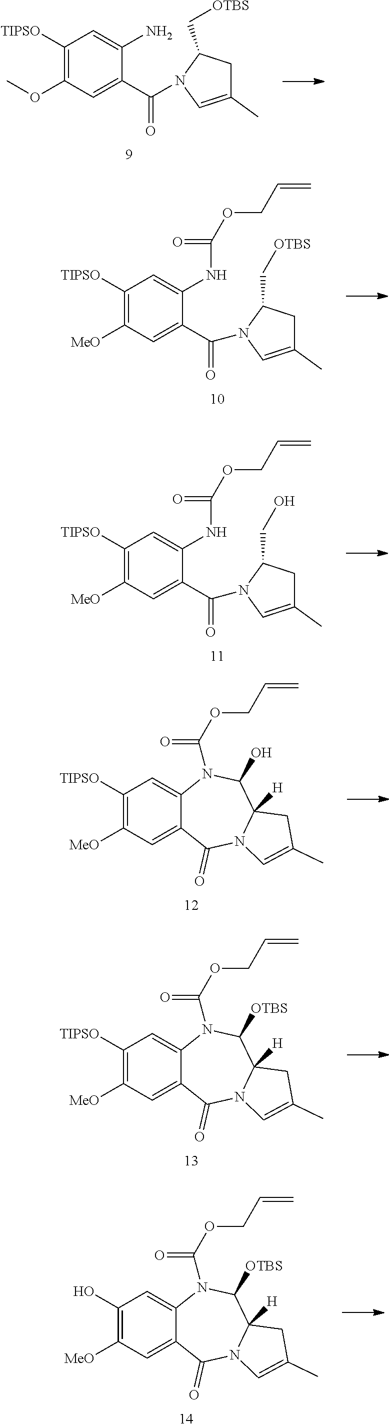

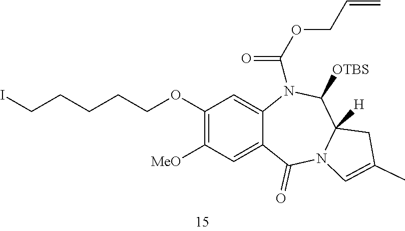 [SMILES: C=CCOC(=O)N1c2cc(OCCCCCI)c(OC)cc2C(=O)N2C=C(C)C[C@H]2[C@@H]1O[Si](C)(C)C(C)(C)C]

### (a) (S)-allyl(2-(2-(((tert-butyldimethylsilyl)oxy)methyl)-4-methyl-2,3-dihydro-1H-pyrrole-)-4-methoxy-5-((triisopropylsilyl)oxy)phenyl)carbamate (10)

Allyl chloroformate (0.30 mL, 3.00 mmol, 1.1 eq) was added to a solution of amine 9 (1.5 g, 2.73 mmol) in the presence of dry pyridine (0.48 mL, 6.00 mmol, 2.2 eq) in dry dichloromethane (20 mL) at −78° C. (acetone/dry ice bath). After 30 minutes, the bath was removed and the reaction mixture was allowed to warm to room temperature. The reaction mixture was diluted with dichloromethane and saturated aqueous copper sulphate was added. The organic layer was then washed sequentially with saturated aqueous sodium bicarbonate and brine. The organic phase was dried over magnesium sulphate, filtered and excess solvent removed by rotary evaporation under reduced pressure to afford the product 10 which was used directly in the next reaction. LC/MS, 4.45 min (ES+) m/z (relative intensity) 632.91 ([M+H]^{+.}, 100)

### (b) (S)-allyl(2-(2-(hydroxymethyl)-4-methyl-2,3-dihydro-1H-pyrrole-)-4-methoxy-5-((triisopropylsilyl)oxy)phenyl)carbamate (11)

The crude 10 was dissolved in a 7:1:1:2 mixture of acetic acid/methanol/tetrahydrofuran/water (28:4:4:8 mL) and allowed to stir at room temperature. After 3 hours, complete disappearance of starting material was observed by LC/MS. The reaction mixture was diluted with ethyl acetate and washed sequentially with water (2×500 mL), saturated aqueous sodium bicarbonate (200 mL) and brine. The organic phase was dried over magnesium sulphate filtered and excess ethyl acetate removed by rotary evaporation under reduced pressure. The resulting residue was subjected to flash column chromatography (silica gel, 25% ethyl acetate in hexane). Pure fractions were collected and combined and excess eluent was removed by rotary evaporation under reduced pressure to afford the desired product 11 (1 g, 71%). LC/MS, 3.70 min (ES+) m/z (relative intensity) 519.13 ([M+H]^{+.}, 95); ¹H NMR (400 MHz, CDCl₃) δ 8.34 (s, 1H), 7.69 (s, 1H), 6.78 (s, 1H), 6.15 (s, 1H), 5.95 (ddt, J=17.2, 10.5, 5.7 Hz, 1H), 5.33 (dq, J=17.2, 1.5 Hz, 1H), 5.23 (ddd, J=10.4, 2.6, 1.3 Hz, 1H), 4.73 (tt, J=7.8, 4.8 Hz, 1H), 4.63 (dt, J=5.7, 1.4 Hz, 2H), 4.54 (s, 1H), 3.89-3.70 (m, 5H), 2.87 (dd, J=16.5, 10.5 Hz, 1H), 2.19 (dd, J=16.8, 4.6 Hz, 1H), 1.70 (d, J=1.3 Hz, 3H), 1.38-1.23 (m, 3H), 1.12 (s, 10H), 1.10 (s, 8H).

### (c) (11S,11aS)-allyl 11-hydroxy-7-methoxy-2-methyl-5-oxo-8-((triisopropylsilyl)oxy)-11,11a-dihydro-1H-benzo[e]pyrrolo[1,2-a][1,4]diazepine-10(5H)-carboxylate (12)

Dimethyl sulphoxide (0.35 mL, 4.83 mmol, 2.5 eq) was added dropwise to a solution of oxalyl chloride (0.2 mL, 2.32 mmol, 1.2 eq) in dry dichloromethane (10 mL) at −78° C. (dry ice/acetone bath) under an atmosphere of argon. After 10 minutes a solution of 11 (1 g, 1.93 mmol) in dry dichloromethane (8 mL) was added slowly with the temperature still at −78° C. After 15 min triethylamine (1.35 mL, dried over 4 Å molecular sieves, 9.65 mmol, 5 eq) was added dropwise and the dry ice/acetone bath was removed. The reaction mixture was allowed to reach room temperature and was extracted with cold hydrochloric acid (0.1 M), saturated aqueous sodium bicarbonate and brine. The organic phase was dried over magnesium sulphate, filtered and excess dichloromethane was removed by rotary evaporation under reduced pressure to afford product 12 (658 mg, 66%). LC/MS, 3.52 min (ES+) m/z (relative intensity) 517.14 ([M+H]^{+.}, 100); ¹H NMR (400 MHz, CDCl₃) δ 7.20 (s, 1H), 6.75-6.63 (m, J=8.8, 4.0 Hz, 2H), 5.89-5.64 (m, J=9.6, 4.1 Hz, 2H), 5.23-5.03 (m, 2H), 4.68-4.38 (m, 2H), 3.84 (s, 3H), 3.83-3.77 (m, 1H), 3.40 (s, 1H), 3.05-2.83 (m, 1H), 2.59 (d, J=17.1 Hz, 1H), 1.78 (d, J=1.3 Hz, 3H), 1.33-1.16 (m, 3H), 1.09 (d, J=2.2 Hz, 9H), 1.07 (d, J=2.1 Hz, 9H).

### (d) (11S,11aS)-allyl 11-((tert-butyldimethylsilyl)oxy)-7-methoxy-2-methyl-5-oxo-8-((triisopropylsilyl)oxy)-11,11a-dihydro-1H-benzo[e]pyrrolo[1,2a-][1,4]diazepine-10(5H)-carboxylate (13)

Tert-butyldimethylsilyltriflate (0.70 mL, 3.00 mmol, 3 eq) was added to a solution of compound 12 (520 mg, 1.00 mmol) and 2,6-lutidine (0.46 mL, 4.00 mmol, 4 eq) in dry dichloromethane (40 mL) at 0° C. under argon. After 10 min, the cold bath was removed and the reaction mixture was stirred at room temperature for 1 hour. The reaction mixture was extracted with water, saturated aqueous sodium bicarbonate and brine. The organic phase was dried over magnesium sulphate, filtered and excess was removed by rotary evaporation under reduced pressure. The resulting residue was subjected to flash column chromatography (silica gel; gradient, 10% ethyl acetate in hexane to 20% ethyl acetate in hexane). Pure fractions were collected and combined and excess eluent was removed by rotary evaporation under reduced pressure to give the product 23 (540 mg, 85%). LC/MS, 4.42 min (ES+) m/z (relative intensity) 653.14 ([M+H]^{+.}, 100); ¹H NMR (400 MHz, CDCl₃) δ 7.20 (s, 1H), 6.71-6.64 (m, J=5.5 Hz, 2H), 5.83 (d, J=9.0 Hz, 1H), 5.80-5.68 (m, J=5.9 Hz, 1H), 5.14-5.06 (m, 2H), 4.58 (dd, J=13.2, 5.2 Hz, 1H), 4.36 (dd, J=13.3, 5.5 Hz, 1H), 3.84 (s, 3H), 3.71 (td, J=10.1, 3.8 Hz, 1H), 2.91 (dd, J=16.9, 10.3 Hz, 1H), 2.36 (d, J=16.8 Hz, 1H), 1.75 (s, 3H), 1.31-1.16 (m, 3H), 1.12-1.01 (m, J=7.4, 2.1 Hz, 18H), 0.89-0.81 (m, 9H), 0.25 (s, 3H), 0.19 (s, 3H).

### (e) (11S,11aS)-allyl 11-((tert-butyldimethylsilyl)oxy)-8-hydroxy-7-methoxy-2-methyl-5-oxo-11,11a-dihydro-M-benzo[e]pyrrolo[1,2-a][1,4]diazepine-10(5H)-carboxylate (24)

Lithium acetate (87 mg, 0.85 mmol) was added to a solution of compound 13 (540 mg, 0.85 mmol) in wet dimethylformamide (6 mL, 50:1 DMF/water). After 4 hours, the reaction was complete and the reaction mixture was diluted with ethyl acetate (25 mL) and washed with aqueous citric acid solution (pH ˜3), water and brine. The organic layer was dried over magnesium sulphate filtered and excess ethyl acetate was removed by rotary evaporation under reduced pressure. The resulting residue was subjected to flash column chromatography (silica gel; gradient, 25% to 75% ethyl acetate in hexane). Pure fractions were collected and combined and excess eluent was removed by rotary evaporation under reduced pressure to give the product 14 (400 mg, quantitative). LC/MS, (3.33 min (ES+) m/z (relative intensity) 475.26 ([M+H]^{+.}, 100).

### (f) (11S,11aS)-allyl 11-((tert-butyldimethylsilyl)oxy)-8-((5-iodopentyl)oxy)-7-methoxy-2-methyl-5-oxo-11,11a-dihydro-M-benzo[e]pyrrolo[1,2-a][1,4]diazepine-10(5H)-carboxylate (15)

Diiodopentane (0.63 mL, 4.21 mmol, 5 eq) and potassium carbonate (116 mg, 0.84 mmol, 1 eq) were added to a solution of phenol 14 (400 mg, 0.84 mmol) in acetone (4 mL, dried over molecular sieves). The reaction mixture was then warmed to 60° C. and stirred for 6 hours. Acetone was removed by rotary evaporation under reduced pressure. The resulting residue was subjected to flash column chromatography (silica gel; 50/50, v/v, hexane/ethyl acetate). Pure fractions were collected and combined and excess eluent was removed to provide 15 in 90% yield. LC/MS, 3.90 min (ES+) m/z (relative intensity) 670.91 ([M]^{+.}, 100). ¹H NMR (400 MHz, CDCl₃) δ 7.23 (s, 1H), 6.69 (s, 1H), 6.60 (s, 1H), 5.87 (d, J=8.8 Hz, 1H), 5.83-5.68 (m, J=5.6 Hz, 1H), 5.15-5.01 (m, 2H), 4.67-4.58 (m, 1H), 4.45-4.35 (m, 1H), 4.04-3.93 (m, 2H), 3.91 (s, 3H), 3.73 (td, J=10.0, 3.8 Hz, 1H), 3.25-3.14 (m, J=8.5, 7.0 Hz, 2H), 2.92 (dd, J=16.8, 10.3 Hz, 1H), 2.38 (d, J=16.8 Hz, 1H), 1.95-1.81 (m, 4H), 1.77 (s, 3H), 1.64-1.49 (m, 2H), 0.88 (s, 9H), 0.25 (s, 3H), 0.23 (s, 3H).

### (iii) (11 S,11 aS)-4-(2-(1-((1-(allyloxy)-4-methyl-1,2-dioxopentan-3-yl)amino)-1-oxopropan-2-yl)hydrazinyl)benzyl 11-((tert-butyldimethylsilyl)oxy)-8-hydroxy-7-methoxy-2-methyl-5-oxo-11,11a-dihydro-1H-benzo[e]pyrrolo[1,2-a][1,4]diazepine-10(5H)-carboxylate (20)

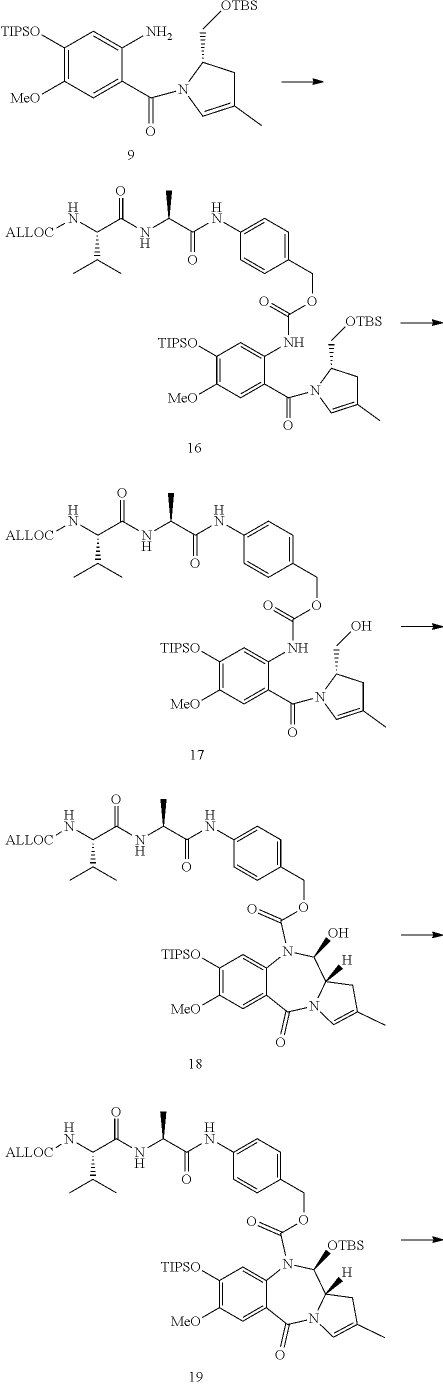

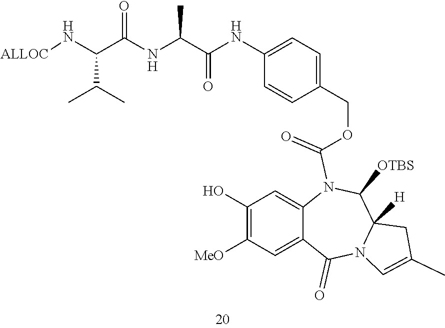

### (a) Allyl 3-(2-(2-(4-((((2-((S)-2-(((tert-butyldimethylsilyl)oxy)methyl)-4-methyl-2,3-dihydro-1H-pyrrole-1-carbonyl)-4-methoxy-5-((triisopropylsilyl)oxy)phenyl)carbamoyl)oxy)methyl)phenyl)hydrazinyl)propanamido)-4-methyl-2-oxopentanoate (16)

Triethylamine (2.23 mL, 18.04 mmol, 2.2 eq) was added to a stirred solution of the amine 9 (4 g, 8.20 mmol) and triphosgene (778 mg, 2.95 mmol, 0.36 eq) in dry tetrahydrofuran (40 mL) at 5° C. (ice bath). The progress of the isocyanate reaction was monitored by periodically removing aliquots from the reaction mixture and quenching with methanol and performing LC/MS analysis. Once the isocyanate formation was complete a solution of the alloc-Val-Ala-PABOH (4.12 g, 12.30 mmol, 1.5 eq) and triethylamine (1.52 mL, 12.30 mmol, 1.5 eq) in dry tetrahydrofuran (40 mL) was rapidly added by injection to the freshly prepared isocyanate. The reaction mixture was allowed to stir at 40° C. for 4 hours. Excess solvent was removed by rotary evaporation under reduced pressure. The resulting residue was subjected to flash column chromatography (silica gel; gradient, 1% methanol to 5% methanol in dichloromethane). (Alternative chromatography conditions using EtOAc and Hexane have also been successful). Pure fractions were collected and combined and excess eluent was removed by rotary evaporation under reduced pressure to give the product 16 (3.9 g, 50%). LC/MS, 4.23 min (ES+) m/z (relative intensity) 952.36 ([M+H]^{+.}, 100); ¹H NMR (400 MHz, CDCl₃) δ 8.62 (br s, 1H), 8.46 (s, 1H), 7.77 (br s, 1H), 7.53 (d, J=8.4 Hz, 2H), 7.32 (d, J=8.5 Hz, 2H), 6.76 (s, 1H), 6.57 (d, J=7.6 Hz, 1H), 6.17 (s, 1H), 6.03-5.83 (m, 1H), 5.26 (dd, J=33.8, 13.5 Hz, 3H), 5.10 (s, 2H), 4.70-4.60 (m, 2H), 4.58 (dd, J=5.7, 1.3 Hz, 2H), 4.06-3.99 (m, 1H), 3.92 (s, 1H), 3.82-3.71 (m, 1H), 3.75 (s, 3H), 2.79-2.64 (m, 1H), 2.54 (d, J=12.9 Hz, 1H), 2.16 (dq, J=13.5, 6.7 Hz, 1H), 1.67 (s, 3H), 1.46 (d, J=7.0 Hz, 3H), 1.35-1.24 (m, 3H), 1.12 (s, 9H), 1.10 (s, 9H), 0.97 (d, J=6.8 Hz, 3H), 0.94 (d, J=6.8 Hz, 3H), 0.87 (s, 9H), 0.07-−0.02 (m, 6H).

### (b) Allyl 3-(2-(2-(4-((((2-((S)-2-(hydroxymethyl)-4-methyl-2,3-dihydro-1H-pyrrole-1-carbonyl)-4-methoxy-5-((triisopropylsilyl)oxy)phenyl)carbamoyl)oxy)methyl)phenyl)hydrazinyl)propanamido)-4-methyl-2-oxopentanoate (17)

The TBS ether 16 (1.32 g, 1.38 mmol) was dissolved in a 7:1:1:2 mixture of acetic acid/methanol/tetrahydrofuran/water (14:2:2:4 mL) and allowed to stir at room temperature. After 3 hours no more starting material was observed by LC/MS. The reaction mixture was diluted with ethyl acetate (25 mL) and washed sequentially with water, saturated aqueous sodium bicarbonate and brine. The organic phase was dried over magnesium sulphate filtered and excess ethyl acetate removed by rotary evaporation under reduced pressure. The resulting residue was subjected to flash column chromatography (silica gel, 2% methanol in dichloromethane). Pure fractions were collected and combined and excess eluent was removed by rotary evaporation under reduced pressure to afford the desired product 17 (920 mg, 80%). LC/MS, 3.60 min (ES+) m/z (relative intensity) 838.18 ([M+H]^{+.}, 100). ¹H NMR (400 MHz, CDCl₃) δ 8.55 (s, 1H), 8.35 (s, 1H), 7.68 (s, 1H), 7.52 (d, J=8.1 Hz, 2H), 7.31 (d, J=8.4 Hz, 2H), 6.77 (s, 1H), 6.71 (d, J=7.5 Hz, 1H), 6.13 (s, 1H), 5.97-5.82 (m, J=5.7 Hz, 1H), 5.41-5.15 (m, 3H), 5.10 (d, J=3.5 Hz, 2H), 4.76-4.42 (m, 5H), 4.03 (t, J=6.6 Hz, 1H), 3.77 (s, 5H), 2.84 (dd, J=16.7, 10.4 Hz, 1H), 2.26-2.08 (m, 2H), 1.68 (s, 3H), 1.44 (d, J=7.0 Hz, 3H), 1.30 (dt, J=14.7, 7.4 Hz, 3H), 1.12 (s, 9H), 1.10 (s, 9H), 0.96 (d, J=6.8 Hz, 3H), 0.93 (d, J=6.8 Hz, 3H).

### (c) (11S,11aS)-4-(2-(1-((1-(allyloxy)-4-methyl-1,2-dioxopentan-3-yl)amino)-1-oxopropan-2-yl)hydrazinyl)benzyl 11-hydroxy-7-methoxy-2-methyl-5-oxo-8-((triisopropylsilyl)oxy)-11,11a-dihydro-M-benzo[e]pyrrolo[1,2-a][1,4]diazepine-10(5H)-carboxylate (18)

Dimethyl sulphoxide (0.2 mL, 2.75 mmol, 2.5 eq) was added dropwise to a solution of oxalyl chloride (0.11 mL, 1.32 mmol, 1.2 eq) in dry dichloromethane (7 mL) at −78° C. (dry ice/acetone bath) under an atmosphere of argon. After 10 minutes a solution of 17 (920 mg, 1.10 mmol) in dry dichloromethane (5 mL) was added slowly with the temperature still at −78° C. After 15 min triethylamine (0.77 mL, dried over 4 Å molecular sieves, 5.50 mmol, 5 eq) was added dropwise and the dry ice/acetone bath was removed. The reaction mixture was allowed to reach room temperature and was extracted with cold hydrochloric acid (0.1 M), saturated aqueous sodium bicarbonate and brine. The organic phase was dried over magnesium sulphate, filtered and excess dichloromethane was removed by rotary evaporation under reduced pressure. The resulting residue was subjected to column flash chromatography (silica gel; gradient 2% methanol to 5% methanol in dichloromethane). Pure fractions were collected and combined and removal of excess eluent by rotary evaporation under reduced pressure afforded the product 18 (550 mg, 60%). LC/MS, 3.43 min (ES+) m/z (relative intensity) 836.01 ([M]^{+.}, 100). ¹H NMR (400 MHz, CDCl₃) δ 8.39 (s, 1H), 7.52-7.40 (m, 2H), 7.21-7.08 (m, J=11.5 Hz, 2H), 6.67 (s, 1H), 6.60-6.47 (m, J=7.4 Hz, 1H), 5.97-5.83 (m, 1H), 5.79-5.66 (m, 1H), 5.38-4.90 (m, 6H), 4.68-4.52 (m, J=18.4, 5.5 Hz, 4H), 4.04-3.94 (m, J=6.5 Hz, 1H), 3.87-3.76 (m, 5H), 3.00-2.88 (m, 1H), 2.66-2.49 (m, 2H), 2.21-2.08 (m, 2H), 1.76 (s, 3H), 1.45 (d, J=7.0 Hz, 3H), 1.09-0.98 (m, J=8.9 Hz, 18H), 0.96 (d, J=6.7 Hz, 3H), 0.93 (d, J=6.9 Hz, 3H).

### (d) (11S,11aS)-4-(2-(1-((1-(Allyloxy)-4-methyl-1,2-dioxopentan-3-yl)amino)-1-oxopropan-2-yl)hydrazinyl)benzyl 11-((tert-butyldimethylsilyl)oxy)-7-methoxy-2-methyl-5-oxo-8-((triisopropylsilyl)oxy)-11,11a-dihydro-M-benzo[e]pyrrolo[1,2-a][1,4]diazepine-10(5H)-carboxylate (19)

Tert-butyldimethylsilyltriflate (0.38 mL, 1.62 mmol, 3 eq) was added to a solution of compound 18 (450 mg, 0.54 mmol) and 2,6-lutidine (0.25 mL, 2.16 mmol, 4 eq) in dry dichloromethane (5 mL) at 0° C. under argon. After 10 min, the cold bath was removed and the reaction mixture was stirred at room temperature for 1 hour. The reaction mixture was extracted with water, saturated aqueous sodium bicarbonate and brine. The organic phase was dried over magnesium sulphate, filtered and excess solvent was removed by rotary evaporation under reduced pressure. The resulting residue was subjected to column flash chromatography (silica gel; 50/50 v/v hexane/ethyl acetate). Pure fractions were collected and combined and excess eluent was removed by rotary evaporation under reduced pressure to give the product 19 (334 mg, 65%). LC/MS, 4.18 min (ES+) m/z (relative intensity) 950.50 ([M]^{+.}, 100). ¹H NMR (400 MHz, CDCl₃) δ 8.53 (s, 1H), 8.02 (s, 1H), 7.44 (d, J=7.6 Hz, 2H), 7.21 (s, 1H), 7.08 (d, J=8.2 Hz, 2H), 6.72-6.61 (m, J=8.9 Hz, 2H), 6.16 (s, 1H), 5.97-5.79 (m, J=24.4, 7.5 Hz, 2H), 5.41-5.08 (m, 5H), 4.86 (d, J=12.5 Hz, 1H), 4.69-4.60 (m, 1H), 4.57 (s, 1H), 4.03 (t, J=6.7 Hz, 1H), 3.87 (s, 3H), 3.74 (td, J=9.6, 3.6 Hz, 1H), 2.43-2.09 (m, J=34.8, 19.4, 11.7 Hz, 3H), 1.76 (s, 3H), 1.43 (d, J=6.9 Hz, 3H), 1.30-1.21 (m, 3H), 0.97 (d, J=6.7 Hz, 3H), 0.92 (t, J=8.4 Hz, 3H), 0.84 (s, 9H), 0.23 (s, 3H), 0.12 (s, 3H).

### (e) (11S,11aS)-4-(2-(1-((1-(Allyloxy)-4-methyl-1,2-dioxopentan-3-yl)amino)-1-oxopropan-2-yl)hydrazinyl)benzyl 11-((tert-butyldimethylsilyl)oxy)-8-hydroxy-7-methoxy-2-methyl-5-oxo-11,11a-dihydro-M-benzo[e]pyrrolo[1,2-a][1,4]diazepine-10(5H)-carboxylate (20)

Lithium acetate (50 mg, 0.49 mmol) was added to a solution of compound 19 (470 mg, 0.49 mmol) in wet dimethylformamide (4 mL, 50:1 DMF/water). After 4 hours, the reaction was complete and the reaction mixture was diluted with ethyl acetate and washed with citric acid (pH ˜3), water and brine. The organic layer was dried over magnesium sulphate filtered and excess ethyl acetate was removed by rotary evaporation under reduced pressure. The resulting residue was subjected to column flash chromatography (silica gel; gradient, 50/50 to 25/75 v/v hexane/ethyl acetate). Pure fractions were collected and combined and excess eluent was removed by rotary evaporation under reduced pressure to give the product 32 (400 mg, quantitative). LC/MS, 3.32 min (ES+) m/z (relative intensity) 794.18 ([M+H]^{+.}, 100). ¹H NMR (400 MHz, CDCl₃) δ 8.53 (s, 1H), 8.02 (s, 1H), 7.44 (d, J=7.6 Hz, 2H), 7.21 (s, 1H), 7.08 (d, J=8.2 Hz, 2H), 6.72-6.61 (m, J=8.9 Hz, 2H), 6.16 (s, 1H), 5.97-5.79 (m, J=24.4, 7.5 Hz, 2H), 5.41-5.08 (m, 5H), 4.86 (d, J=12.5 Hz, 1H), 4.69-4.60 (m, 1H), 4.57 (s, 1H), 4.03 (t, J=6.7 Hz, 1H), 3.87 (s, 3H), 3.74 (td, J=9.6, 3.6 Hz, 1H), 2.43-2.09 (m, J=34.8, 19.4, 11.7 Hz, 3H), 1.76 (s, 3H), 1.43 (d, J=6.9 Hz, 3H), 1.30-1.21 (m, 3H), 0.97 (d, J=6.7 Hz, 3H), 0.92 (t, J=8.4 Hz, 3H), 0.84 (s, 9H), 0.23 (s, 3H), 0.12 (s, 3H).

### (iv) (11S,11aS)-4-((2S,5S)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl 11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (24)

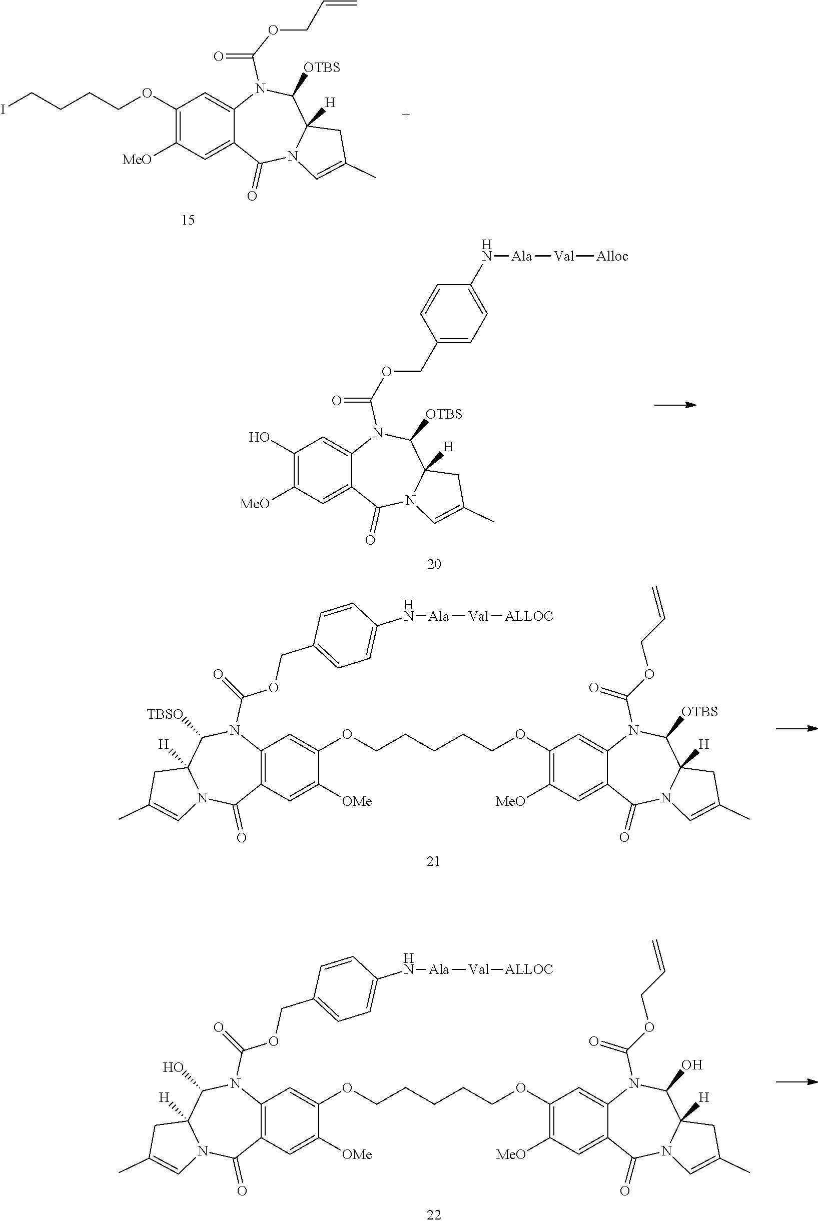

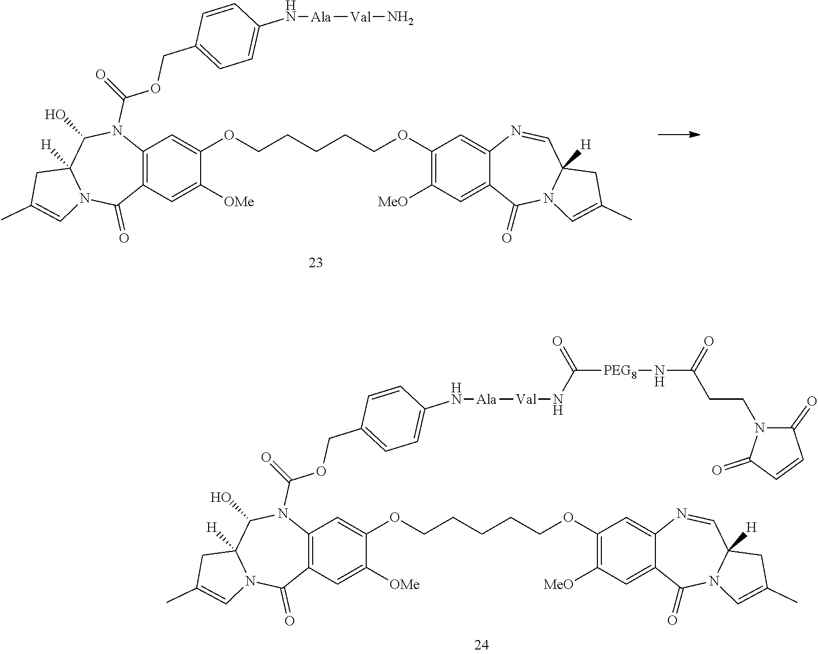

### (a) (11S)-allyl 8-((5-(((11S)-10-(((4-(2-(1-((1-(allyloxy)-4-methyl-1,2-dioxopentan-3-yl)amino)-1-oxopropan-2-yl)hydrazinyl)benzyl)oxy)carbonyl)-11-((tert-butyldimethylsilyl)oxy)-7-methoxy-2-methyl-5-oxo-5,10,11,11a-tetrahydro-1H-benzo[e]pyrrolo[1,2-a][1,4]diazepin-8-yl)oxy)pentyl)oxy)-11-((tert-butyldimethylsilyl)oxy)-7-methoxy-2-methyl-5-oxo-11,11a-dihydro-1H-benzo[e]pyrrolo[1,2-a][1,4]diazepine-10(5H)-carboxylate (21)

Potassium carbonate (70 mg, 0.504 mmol, 1 eq) was added to a solution of (370 mg, 0.552 mmol, 1.2 eq) and phenol 20 (400 mg, 0.504 mmol) in dry acetone (25 mL). The reaction was stirred 8 hours at 70° C. The LC/MS showed that all the starting material was not consumed, so the reaction was allowed to stir overnight at room temperature and stirred for an additional 2 hours the next day. Acetone was removed by rotary evaporation under reduced pressure. The resulting residue was subjected to flash column chromatography (silica gel; 80% ethyl acetate in hexane to 100% ethyl acetate). Pure fractions were collected and combined and excess eluent was removed by rotary evaporation under reduced pressure to give the product 21 (385 mg, 57%). LC/MS, 4.07 min (ES+) m/z (relative intensity) 1336.55 ([M+H]^{+.}, 50).

### (b) (11S)-allyl 8-((5-(((11S)-10-(((4-(2-(1-((1-(allyloxy)-4-methyl-1,2-dioxopentan-3-yl)amino)-1-oxopropan-2-yl)hydrazinyl)benzyl)oxy)carbonyl)-11-hydroxy-7-methoxy-2-methyl-5-oxo-5,10,11,11a-tetrahydro-1H-benzo[e]pyrrolo[1,2-a][1,4]diazepin-8-yl)oxy)pentyl)oxy)-11-hydroxy-7-methoxy-2-methyl-5-oxo-11,11a-dihydro-1H-benzo[e]pyrrolo[1,2-a][1,4]diazepine-10(5H)-carboxylate (22)

Tetra-n-butylammonium fluoride (1M, 0.34 mL, 0.34 mmol, 2 eq) was added to a solution of 21 (230 mg, 0.172 mmol) in dry tetrahydrofuran (3 mL). The starting material was totally consumed after 10 minutes. The reaction mixture was diluted with ethyl acetate (30 mL) and washed sequentially with water and brine. The organic phase was dried over magnesium sulphate filtered and excess ethyl acetate removed by rotary evaporation under reduced pressure. The resulting residue 22 was used as a crude mixture for the next reaction. LC/MS, 2.87 min (ES+) m/z (relative intensity) 1108.11 ([M+H]^{+.}, 100).

### (c) (11S)-4-(2-(1-((1-amino-3-methyl-1-oxobutan-2-yl)amino)-1-oxopropan-2-yl)hydrazinyl)benzyl 11-hydroxy-7-methoxy-8-((5-((7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (23)

Tetrakis(triphenylphosphine)palladium(0) (12 mg, 0.01 mmol, 0.06 eq) was added to a solution of crude 22 (0.172 mmol) and pyrrolidine (36 μL, 0.43 mmol, 2.5 eq) in dry dichloromethane (10 mL). The reaction mixture was stirred 20 minutes and diluted with dichloromethane and washed sequentially with saturated aqueous ammonium chloride and brine. The organic phase was dried over magnesium sulphate filtered and excess dichloromethane removed by rotary evaporation under reduced pressure. The resulting residue 23 was used as a crude mixture for the next reaction. LC/MS, 2.38 min (ES+) m/z (relative intensity) 922.16 ([M+H]^{+.}, 40).

### (d) (11S,11aS)-4-((2S,5S)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl 11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (24)

1-ethyl-3-(3′-dimethylaminopropyl)carbodiimide (EDCI, 33 mg, 0.172 mmol) was added to a solution of crude 23 (0.172 mmol) and Mal-(PEG)₈-acid (100 mg, 0.172 mmol) in dry dichloromethane (10 mL). The reaction was stirred for 2 hours and the presence of starting material was no longer observed by LC/MS. The reaction was diluted with dichloromethane and washed sequentially with water and brine. The organic phase was dried over magnesium sulphate filtered and excess dichloromethane removed by rotary evaporation under reduced pressure. The resulting residue was subjected to flash column chromatography (silica gel; 100% chloroform to 10% methanol in chloroform). Pure fractions were collected and combined and excess eluent was removed by rotary evaporation under reduced pressure to give 24 (B) (60 mg, 25% over 3 steps).

7.3: Synthesis of Drug Moiety 33 (Referred Hereinafter as “33”)

### (i) (11 S,11aS)-4-((S)-2-((S)-2-(((allyloxy)carbonyl)amino)-3-methylbutanamido)-5-ureidopentanamido)benzyl 11-((tert-butyldimethylsilyl)oxy)-8-hydroxy-7-methoxy-2-methyl-5-oxo-11,11a-dihydro-1H-benzo[e]pyrrolo[1,2-a][1,4]diazepine-10(5H)-carboxylate (29)

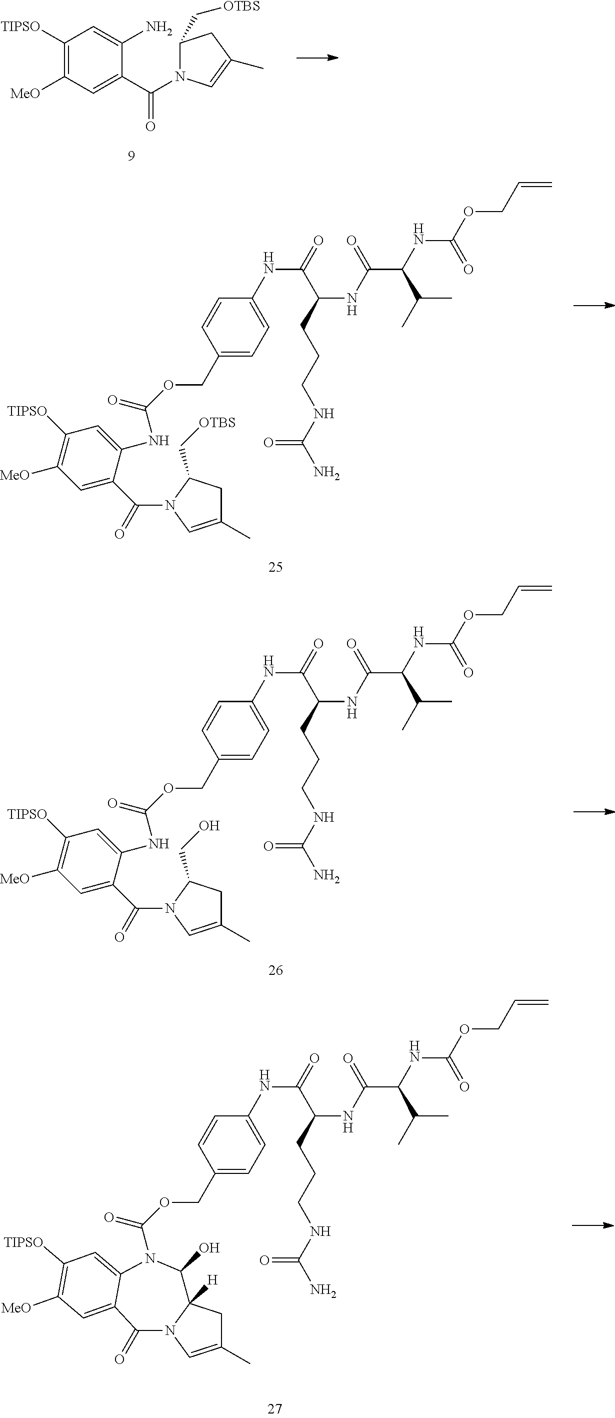

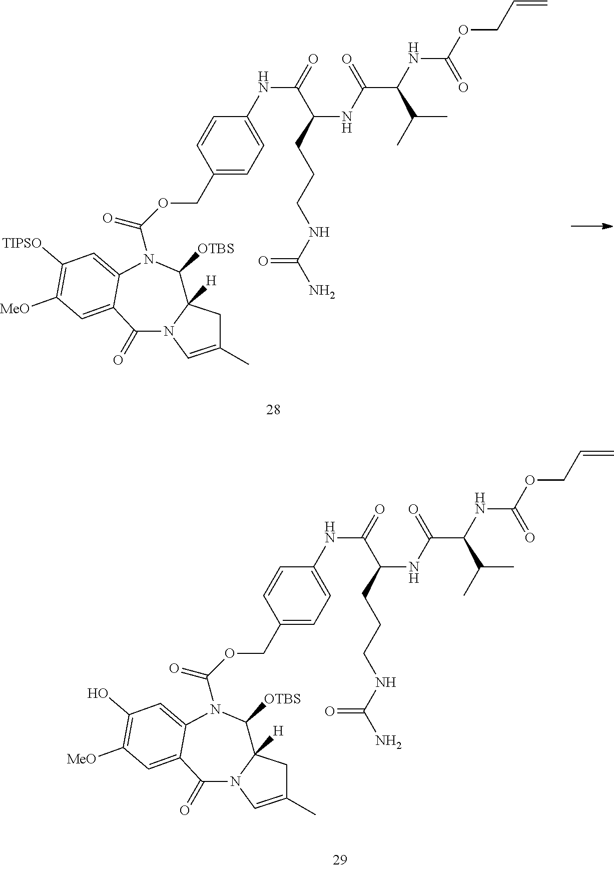

### (a) 4-((S)-2-((S)-2-(((allyloxy)carbonyl)amino)-3-methylbutanamido)-5-ureidopentanamido)benzyl(2-((S)-2-(((tert-butyldimethylsilyl)oxy)methyl)-4-methyl-2,3-dihydro-1H-pyrrole-1-carbonyl)-4-methoxy-5-((triisopropylsilyl)oxy)phenyl)carbamate (25)

Triethylamine (1.07 mL, 7.69 mmol, 2.5 eq) was added to a stirred solution of the amine 9 (1.69 g, 3.08 mmol) and triphosgene (329 mg, 1.11 mmol, 0.36 eq) in dry tetrahydrofuran (20 mL) at 0° C. (ice bath). The progress of the isocyanate reaction was monitored by periodically removing aliquots from the reaction mixture and quenching with methanol and performing LC/MS analysis. Once the isocyanate formation was complete a solution of the alloc-Val-Cit-PABOH (1.85 g, 4.00 mmol, 1.3 eq) and triethylamine (0.56 mL, 4.00 mmol, 1.5 eq) in dry tetrahydrofuran (40 mL) was rapidly added by injection to the freshly prepared isocyanate. The reaction mixture was allowed to stir at 40° C. for 4 hours. Excess solvent was removed by rotary evaporation under reduced pressure. The resulting residue was subjected to flash column chromatography (silica gel; gradient, 1% methanol to 5% methanol in chloroform). Pure fractions were collected and combined and excess eluent was removed by rotary evaporation under reduced pressure to give the product 25 (0.98 g, 31%). LC/MS, 4.13 min (ES+) m/z (relative intensity) 1038.39 ([M+H]^{+.}, 100); ¹H NMR (400 MHz, DMSO-d6) δ 10.07 (s, 1H), 9.00 (br s, 1H), 8.11 (d, J=8 Hz, 1H), 7.64 (d, J=8 Hz, 2H), 7.33 (d, J=8 Hz, 2H), 7.25 (d, J=8 Hz, 2H), 6.85 (s, 1H), 6.06-5.90 (m, 3H), 5.42 (s, 2H), 5.34 (d, J=16 Hz, 1H), 5.21 (d, J=8 Hz, 1H), 5.06 (s, 2H), 4.52-4.45 (m, 4H), 3.97-3.85 (m, 2H), 3.77 (m, 4H), 3.05-2.99 (m, 2H), 2.68 (m, 1H), 2.43 (m, 1H), 2.01 (m, 1H), 1.69-1.65 (m, 5H), 1.46 (m, 2H), 1.28-1.24 (m, 2H), 1.10 (s, 9H), 1.09 (s, 9H), 0.87 (m, 12H), 0.07-0.06 (m, 6H).

### (b) 4-((S)-2-((S)-2-(((allyloxy)carbonyl)amino)-3-methylbutanamido)-5-ureidopentanamido)benzyl(2-((S)-2-(hydroxymethyl)-4-methyl-2,3-dihydro-1H-pyrrole-1-carbonyl)-4-methoxy-5-((triisopropylsilyl)oxy)phenyl)carbamate (26)

The TBS ether 25 (1.88 g, 1.81 mmol) was dissolved in a 7:1:1:2 mixture of acetic acid/methanol/tetrahydrofuran/water (21:3:3:6 mL) and allowed to stir at room temperature. After 2 hours no more starting material was observed by LC/MS. The reaction mixture was diluted with ethyl acetate (50 mL) and washed sequentially with water, saturated aqueous sodium bicarbonate and brine. The organic phase was dried over magnesium sulphate filtered and excess ethyl acetate removed by rotary evaporation under reduced pressure. The resulting residue was subjected to flash column chromatography (silica gel, 1% methanol to 5% methanol in chloroform). Pure fractions were collected and combined and excess eluent was removed by rotary evaporation under reduced pressure to afford the desired product 26 (877 mg, 53%). LC/MS, 3.43 min (ES+) m/z (relative intensity) 924.05 ([M+H]^{+.}, 100). ¹H NMR (400 MHz, DMSO-d6) δ 10.07 (s, 1H), 8.99 (br s, 1H), 8.11 (d, J=8 Hz, 1H), 7.64 (d, J=8 Hz, 2H), 7.34 (d, J=8 Hz, 2H), 7.26 (d, J=8 Hz, 2H), 6.91 (s, 1H), 6.05-5.90 (m, 3H), 5.43 (s, 2H), 5.34 (d, J=16 Hz, 1H), 5.21 (d, J=8 Hz, 1H), 5.06 (s, 2H), 4.87 (m, 1H), 4.53-4.45 (m, 4H), 3.95 (m, 1H), 3.78 (s, 3H), 3.67 (m, 1H), 3.58 (m, 1H), 3.09-2.96 (m, 2H), 2.69 (m, 1H), 2.44 (m, 1H), 2.02 (m, 1H), 1.73-1.63 (m, 5H), 1.43 (m, 2H), 1.27 (m, 3H), 1.10 (s, 9H), 1.08 (s, 9H), 0.89 (dd, J=4 Hz, 12H).

### (c) (11S,11aS)-4-((S)-2-((S)-2-(((allyloxy)carbonyl)amino)-3-methylbutanamido)-5-ureidopentanamido)benzyl 11-hydroxy-7-methoxy-2-methyl-5-oxo-8-((triisopropylsilyl)oxy)-11,11a-dihydro-M-benzo[e]pyrrolo[1,2-a][1,4]diazepine-10(5H)-carboxylate (27)

SIBX (0.678 g, 1.09 mmol) was added to a stirred solution of 26 (0.840 g, 0.909 mmol) in anhydrous DMF (15 mL) for 96 h at room temperature under Ar. Reaction mixture diluted with water (30 mL), extracted into 10% MeOH/DCM, organic layer washed with saturated aqueous sodium bicarbonate and brine. The organic phase was dried over magnesium sulphate filtered and excess MeOH/DCM removed by rotary evaporation under reduced pressure. The resulting residue was subjected to flash column chromatography (silica gel, 1% methanol to 5% methanol in chloroform). Pure fractions were collected and combined and excess eluent was removed by rotary evaporation under reduced pressure to afford the desired product 27 (120 mg, 12%). LC/MS, 7.55 min (ES+) m/z (relative intensity) 922.68 ([M+H]^{+.}, 100).

### (d) (11S,11aS)-4-((S)-2-((S)-2-(((allyloxy)carbonyl)amino)-3-methylbutanamido)-5-ureidopentanamido)benzyl 11-((tert-butyldimethylsilyl)oxy)-7-methoxy-2-methyl-5-oxo-8-((triisopropylsilyl)oxy)-11,11a-dihydro-M-benzo[e]pyrrolo[1,2-a][1,4]diazepine-10(5H)-carboxylate (28)

Tert-butyldimethylsilyltriflate (0.08 mL, 0.33 mmol, 3 eq) was added to a solution of compound 27 (102 mg, 0.11 mmol) and 2,6-lutidine (0.05 mL, 0.44 mmol, 4 eq) in dry dichloromethane (1.5 mL) at 0° C. under argon. After 10 min, the cold bath was removed and the reaction mixture was stirred at room temperature for 1 hour. The reaction mixture was extracted with water, saturated aqueous sodium bicarbonate and brine. The organic phase was dried over magnesium sulphate, filtered and excess solvent was removed by rotary evaporation under reduced pressure. The resulting crude product was used in the next step. LC/MS, 4.07 min (ES+) m/z (relative intensity) 1036.07 ([M]^{+.}, 100).

### (e) (11S,11aS)-4-((S)-2-((S)-2-(((allyloxy)carbonyl)amino)-3-methylbutanamido)-5-ureidopentanamido)benzyl 11-((tert-butyldimethylsilyl)oxy)-8-hydroxy-7-methoxy-2-methyl-5-oxo-11,11a-dihydro-M-benzo[e]pyrrolo[1,2-a][1,4]diazepine-10(5H)-carboxylate (29)

Lithium acetate (21 mg, 0.20 mmol) was added to a solution of compound 28 (assumed 100%, 0.20 mmol) in wet dimethylformamide (2 mL, 50:1 DMF/water). After 4 hours, the reaction was complete and the reaction mixture was diluted with ethyl acetate and washed with citric acid (pH ˜3), water and brine. The organic layer was dried over magnesium sulphate filtered and excess ethyl acetate was removed by rotary evaporation under reduced pressure. The resulting crude product was used in the next step. LC/MS, 3.15 min (ES+) m/z (relative intensity) 880.45 ([M+H]^{+.}, 100).

### (ii) (11S,11aS)-4-((2S,5S)-37-(2,5-dioxo-2,5-dihydro-1H-pyrrol-1-yl)-5-isopropyl-4,7,35-trioxo-2-(3-ureidopropyl)-10,13,16,19,22,25,28,31-octaoxa-3,6,34-triazaheptatriacontanamido)benzyl 11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (33)

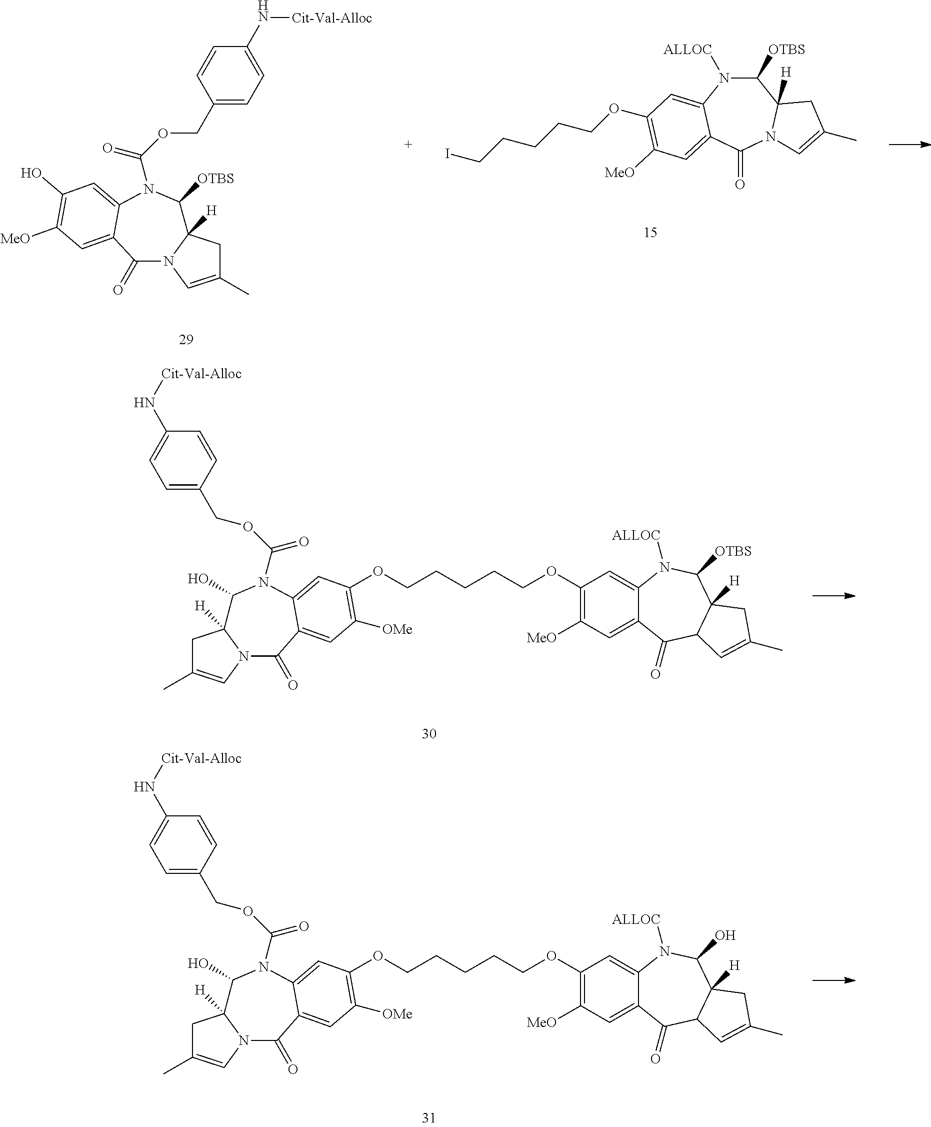

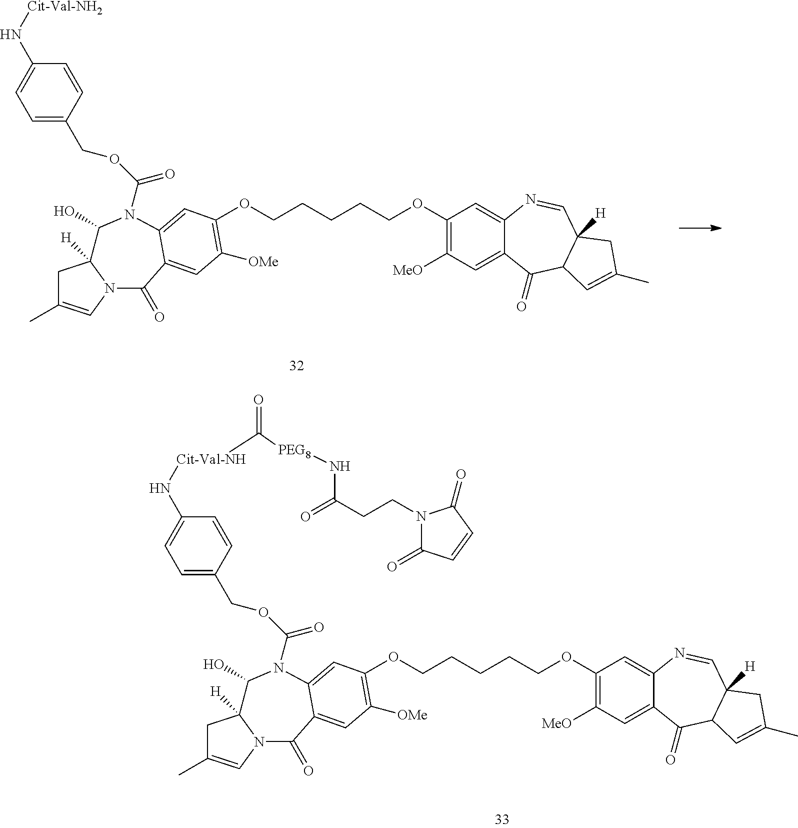

### (a) (11S,11aS)-4-((S)-2-((S)-2-(((allyloxy)carbonyl)amino)-3-methylbutanamido)-5-ureidopentanamido)benzyl 8-((5-(((3aR,4S)-5-((allyloxy)carbonyl)-4-((tert-butyldimethylsilyl)oxy)-8-methoxy-2-methyl-10-oxo-3,3a,4,5,10,10a-hexahydrobenzo[b]cyclopenta[e]azepin-7-yl)oxy)pentyl)oxy)-11-((tert-butyldimethylsilyl)oxy)-7-methoxy-2-methyl-5-oxo-11,11a-dihydro-M-benzo[e]pyrrolo[1,2-a][1,4]diazepine-10(5H)-carboxylate (30)

Potassium carbonate (21 mg, 0.16 mmol, 0.8 eq) was added to a solution of 15 (130 mg, 0.194 mmol, 1 eq) and phenol 29 (assumed 100%, 0.194 mmol) in dry acetone (3 mL). The reaction was stirred 2.5 hours at 70° C. Acetone was removed by rotary evaporation under reduced pressure. The resulting residue was subjected to flash column chromatography (silica gel; 100% chloroform to 4% methanol). Pure fractions were collected and combined and excess eluent was removed by rotary evaporation under reduced pressure to give the product 30 (29 mg, 11%). LC/MS, 4.00 min (ES+) m/z (relative intensity) 1423.30 ([M+H]^{+.}, 100).

### (b) (11S,11aS)-4-((S)-2-((S)-2-(((allyloxy)carbonyl)amino)-3-methylbutanamido)-5-ureidopentanamido)benzyl 8-((5-(((3aR,4S)-5-((allyloxy)carbonyl)-4-hydroxy-8-methoxy-2-methyl-10-oxo-3,3a,4,5,10,10a-hexahydrobenzo[b]cyclopenta[e]azepin-7-yl)oxy)pentyl)oxy)-11-hydroxy-7-methoxy-2-methyl-5-oxo-11,11a-dihydro-M-benzo[e]pyrrolo[1,2-a][1,4]diazepine-10(5H)-carboxylate (31)

Tetra-n-butylammonium fluoride (1M, 0.04 mL, 0.04 mmol, 2 eq) was added to a solution of 30 (29 mg, 0.02 mmol) in dry tetrahydrofuran (1.5 mL). The starting material was totally consumed after 10 minutes. The reaction mixture was diluted with dichloromethane (25 mL) and washed sequentially with water and brine. The organic phase was dried over magnesium sulphate filtered and excess dichloromethane removed by rotary evaporation under reduced pressure. The resulting residue 31 was used as a crude mixture for the next reaction. LC/MS, 2.75 min (ES+) m/z (relative intensity) 1193.93 ([M+H]^{+.}, 100).

### (c) (11S,11aS)-4-((S)-2-((S)-2-amino-3-methylbutanamido)-5-ureidopentanamido)benzyl 11-hydroxy-7-methoxy-8-((5-(((3aR)-8-methoxy-2-methyl-10-oxo-3,3a,10,10a-tetrahydrobenzo[b]cyclopenta[e]azepin-7-yl)oxy)pentyl)oxy)-2-methyl-5-oxo-11,11a-dihydro-1H-benzo[e]pyrrolo[1,2-a][1,4]diazepine-10(5H)-carboxylate (32)

Tetrakis(triphenylphosphine)palladium(0) (1.5 mg, 0.001 mmol, 0.06 eq) was added to a solution of crude 31 (assumed 100%, 0.02 mmol) and pyrrolidine (4.2 μL, 0.05 mmol, 2.5 eq) in dry dichloromethane (2 mL). The reaction mixture was stirred 40 minutes and diluted with dichloromethane and washed sequentially with saturated aqueous ammonium chloride and brine. The organic phase was dried over magnesium sulphate filtered and excess dichloromethane removed by rotary evaporation under reduced pressure. The resulting residue 32 was used as a crude mixture for the next reaction. LC/MS, 2.35 min (ES+) m/z (relative intensity) 1008.22 ([M+H]^{+.}, 100).

### (d) (11S,11aS)-4-((2S,5S)-37-(2,5-dioxo-2,5-dihydro-1H-pyrrol-1-yl)-5-isopropyl-4,7,35-trioxo-2-(3-ureidopropyl)-10,13,16,19,22,25,28,31-octaoxa-3,6,34-triazaheptatriacontanamido)benzyl 11-hydroxy-7-methoxy-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-11,11a-dihydro-1H-benzo[e]pyrrolo[1,2-a][1,4]diazepine-10(5H)-carboxylate (33)

1-ethyl-3-(3′-dimethylaminopropyl)carbodiimide (EDCI, 3.9 mg, 0.02 mmol) was added to a solution of crude 32 (assumed 100%, 0.02 mmol) and Mal-(PEG)₈-acid (12.1 mg, 0.02 mmol) in dry dichloromethane (1.5 mL). The reaction was stirred for 1 hour and the presence of starting material was no longer observed by LC/MS. The reaction mixture was evaporated and the resulting residue was subjected to preparative HPLC (mobile phase of water [A] [formic acid 0.1%] and acetonitrile [B] [formic acid 0.1%]. Gradient: initial composition 100% A to 100% B over 15.0 min, held for 2.0 min at 100% B, and then returned to 13% B in 0.1 minutes and held there for 2.9 min. Total gradient run time equals 20 min, flow rate 20 mL/min). Pure fractions were collected and combined and excess eluent was removed by lyophilisation to give 33 (1.6 mg, 5% over 3 steps).

### Example 8

### Preparation of ADCs with PBD Dimers

Antibodies (5 mg/ml) were partially reduced with Tris(2-carboxyethyl)phosphine hydrochloride (TCEP) in 10 mM borate buffer pH 8.4 containing 150 mM NaCl and 2 mM EDTA for 2 h at 37° C. Typically, 1.5 and 3 molar equivalents of TCEP were used to target Drug-to-Antibody Ratios (DARs) of about 2 and 4, respectively. The concentration of free thiol residues was determined by titrating with 5,5′-dithiobis(2-nitrobenzoic acid) (DTNB, Ellman's reagent), typically resulting in around 3 and 5 thiols released per antibody after TCEP treatments performed to target DARs of 2 and 4, respectively. The partial antibody reduction was also confirmed by SDS-PAGE analysis under non reducing conditions. Before drug coupling to the released interchain cysteine residues, the reduction mixture was allowed to cool to room temperature. The antibody concentration was then adjusted to 1 mg/ml with 10 mM borate buffer pH 8.4 containing 150 mM NaCl and 2 mM EDTA, and a 1.5 to 2 molar excess of drug to reactive thiol groups was added from a 10 mM solution in dimethyl sulfoxide (DMSO). The final DMSO concentration was adjusted to 10% to maintain the solubility of the drug in the aqueous medium during coupling. The reaction was carried out for 1 h at room temperature. A sample of the reaction mixture was taken and used to estimate the residual free thiols by using DTNB before quenching the reaction. The drug excess was quenched by addition of 1.5 moles of N-acetylcysteine per mole of drug and incubation for 1 h at room temperature. After dialysis against 25 mM His buffer pH 6.5 containing 150 mM NaCl overnight at 4° C., the antibody drug conjugates were purified by using methods known to persons skilled in the art based with commercial chromatography columns and ultrafiltration units. First, the non coupled drug and the ADC aggregates were eliminated by size exclusion chromatography (SEC) on S200 (GE Life Sciences) or TSK G3000 SW (Tosoh) column. The purified ADC monomers were then concentrated to 2-3 mg/ml by ultrafiltration on 30 or 50 kDa MWCO filtration units or by affinity chromatography on Protein A. The purified ADCs were stored at 4° C. after sterile filtration on 0.2 μm filter. They were further analyzed by SDS-PAGE under reducing and non reducing conditions to confirm drug conjugation and by SEC on analytical S200 or TSK G3000 SWXL columns to determine the content of monomers and aggregated forms. Protein concentrations were determined by using the bicinchoninic acid assay (BCA) with IgG as a standard.

The DAR was estimated for each ADC by calculating the difference of the number of free thiols determined after the drug coupling and mild reduction steps by titration using the reagent DTNB. Typically, the DAR determined by using this method was comprised between 3.4 and 4.9 (mean value of 3.9) for a targeted DAR of 4, and between 1.2 and 2.1 (mean value of 1.8) for a targeted DAR of 2. The content of aggregated forms was lower than 5% after purification.

Preferred ADC according to the invention are i) ADC comprising the hz1613F12 linked to the Drug Moiety 24 (referred as hz1613F12-24) and ii) ADC comprising the hz1613F12 linked to the Drug Moiety 33 (referred as hz1613F12-33). The Drug-Antibody Ratio is stipulated after the name of the ADC by the expression “DAR X” wherein X corresponds to the said ratio.

### Example 9

### Determination of the ADCs of the Invention Binding on Axl Receptor after Drug Linker Conjugation

Binding assays are commonly used to characterize the activity of a product through binding to its specific receptor. In the present example, FACS analysis was performed to establish if the conjugation process and the presence of the grafted linker drug alter the ability of the resulting ADC to bind target antigen. So binding of the naked hz1613F12 with those of the ADCs of the invention was compared: first, in flow cytometry experiment with SN12C human tumor renal cells and secondly, in ELISA on rhAxl immobilized protein.

9.1 Validation of hz1613F12-24 DAR4 and DAR 2 Binding on Cell-Surface Axl Receptor by Flow Cytometry (FACS)

The FACS experiment was performed as described hereinafter. Briefly, confluent SN12C cells were detached with 1 ml of Trypsin-EDTA for 5 min and then resuspended in complete growth medium. Cell concentration and viability were determined with a Vicell instrument using Trypan-blue exclusion method. Cell concentration was adjusted at 10⁶ cells/ml and the staining was performed in 10⁵ cells. Two-fold serial dilutions (from 6.67 10⁻⁸ M to 6.5 10⁻¹¹ M) of hz1613F12 or hz1613F12-24 DAR4 or DAR2 were added to the cells and left at 4° C. for 20 min. The cells were washed twice with 100 μl of FACS buffer (phosphate-buffered saline (PBS) supplemented with 1% BSA and 0.01% NaN₃). Alexa Fluor® 488 Goat Anti-Human IgG (H+L) (Invitrogen, Al 1013, 1:500) was added and cells were stained for 20 min at 4° C. Cells were washed twice as described before and resuspended in 100 μl of FACS buffer for flow cytometric analysis. Prior to the sample analysis, propidium iodide is added to the cell samples. A Becton Dickinson Facscalibur instrument using 488 argon lasers was used. Data were then analysed using Prism application.

Results are presented in FIG. 5.

Data show that similar EC₅₀ value of binding of hz1613F12 and of hz1613F12-24 DAR4 and DAR2 are obtained.

9.2 Validation of hz1613F12-24 DAR4 and DAR2 Binding on rhAxl Extracellular Domain by ELISA

In this example, the binding of hz1613F12 and of both hz1613F12-24 DAR4 and DAR2 was compared on the immobilized rhAxl-Fc protein by ELISA.

Briefly, the recombinant human Axl-Fc (R and D Systems, cat N° 154AL/CF) protein was coated overnight at 4° C. to Immulon II 96-well plates and, after a 1 h blocking step with a 0.5% gelatine solution, 1613F12 or hz1613F12-24 ADCs to be tested were added for an additional 1 h at 37° C. at starting concentration of 3.33 10⁻⁸M. Then two-fold serial dilutions were done over 12 columns. Plates were washed and a HRP coupled-goat anti-human Kappa light chain (Sigma, ref. A7164, 1/5000°) was added for 1 h at 37° C. Reaction development was performed using the TMB substrate solution.

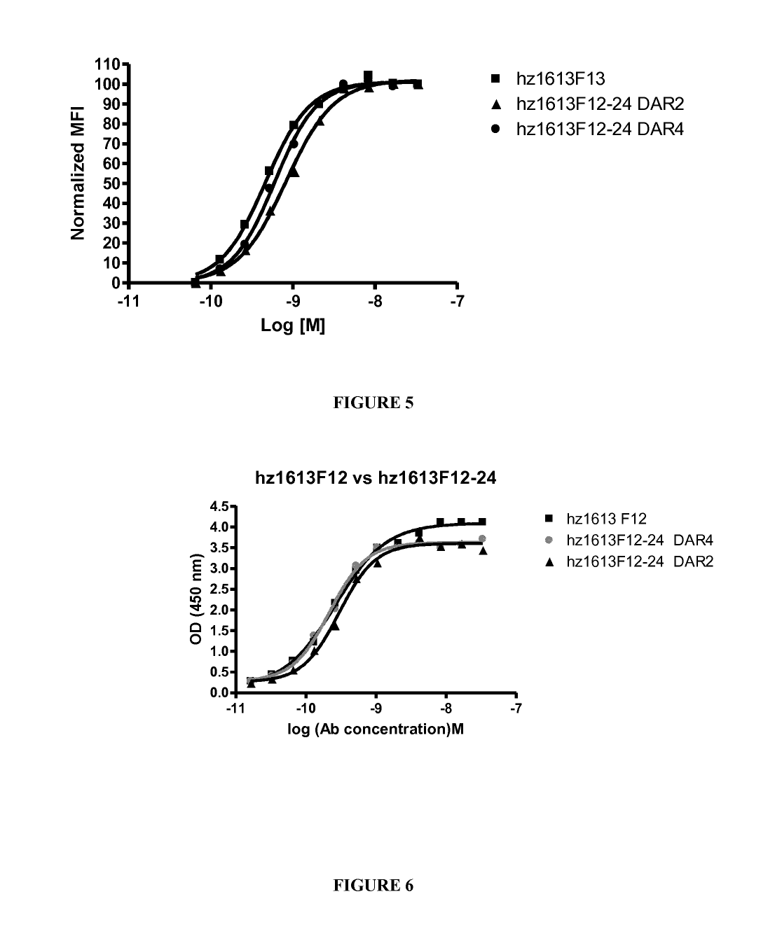

Results are represented in FIG. 6.

Data show that similar EC₅₀ value of binding of hz1613F12 and of hz1613F12-24 DAR4 and DAR2 ADCs are obtained. This example confirms that the conjugation of the Drug Moiety 24 on the free cysteine residues of hz1613F12 doesn't affect binding ability of the ADC to its target.

9.3 Validation of hz1613F12-33 DAR4 ADC Binding on Cell-Surface Axl Receptor by Flow Cytometry (FACS)

The FACS experiment was performed as described above in 9.1 except that the ADC is hz1613F12-33.

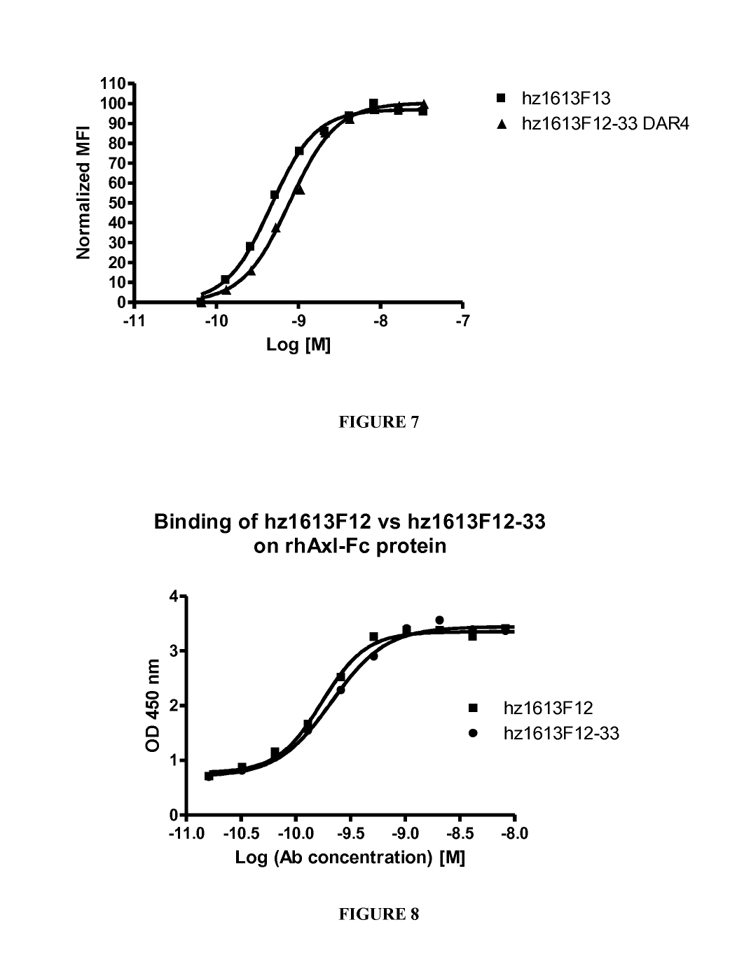

Results are presented in FIG. 7.

Data show that drug coupling did not affect ADC binding on SN12C cells as EC₅₀ are very close.

9.4 Validation of hz1613F12-33 DAR4 ADC Binding on rhAxl Extracellular Domain by ELISA

In this example, the binding of hz1613F12 and of hz1613F12-33 DAR4 was compared on the immobilized rhAxl-Fc protein by ELISA. The protocol is given above in 9.2, except that the used ADC is hz1613F12-33.

Results are represented in FIG. 8.

Prism analysis revealed that the EC₅₀ values of binding for hz1613F12-33 DAR4 are comparable to those of the unconjugated hz1613F12.

This example confirms that the conjugation of the Drug Moiety 33 on the reduced cysteine residues of hz1613F12 doesn't affect binding ability of the ADC to its target.

### Example 10

### Cytotoxic Activity of hz1613F12-PBD ADC on a Panel of Human Tumor Cells

In the present invention, hz1613F12 is coupled to Drug Moiety 24 and 33 to form ADC compounds. The nature of the linkers used may vary. A list of the putative linkers was described above. However a potent cytotoxic activity of the resulting ADC can be obtained with various linkers.

10.1. In Vitro Cytotoxic Activity of hz1613F12-24 DAR4 on a Panel of Human Tumor Cell Lines.

First, once coupled to the PBD drug linker compound, the cytotoxic activity of the resulting ADC hz1613F12-24 DAR4 (preparation described in Example 8) was assessed in in vitro cellular assays as described bellow. The ADC was tested against a panel of human tumor cell lines expressing various levels of cell-surface Axl as well as against a control cell line, MCF7.

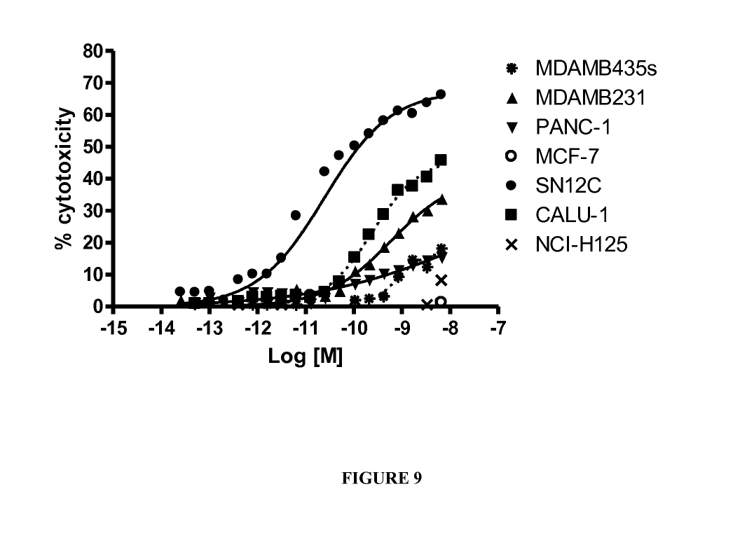

Briefly, human tumor cells were plated for 24 hours in complete culture medium in mw96 plates. The day after, increasing concentrations of hz1612F12-24 DAR4 were added. Triplicate wells were prepared for each condition. Following the addition of the antibody drug conjugate, cells were incubated for 3 days at 37° C. Cell viability was assessed using CellTiter-Glo® Luminescent Cell Viability Assay (Promega; Madison; USA) according to manufacturer's protocol. Percentage of cytotoxicity was determined for each concentration of antibody drug conjugate (FIG. 9).

Data were then analyzed using Prism application in order to determine EC₅₀ value for each tested antibody drug conjugate and are joined in the following table 5.

TABLE 5

| | | % Max |
|---|---|---|
| Cells | EC₅₀ | cytotoxicity |
| MDA-MB435s | 7.9 × 10⁻¹⁰M | 18% |
| MDA-MB231 | 5.9 × 10⁻¹⁰M | 32% |
| SN12C | 2.4 × 10⁻¹¹M | 70% |
| CALU-1 | 2.5 × 10⁻¹⁰M | 40% |
| PANC-1 | 8.7 × 10⁻¹¹M | 15% |

Data in FIG. 9 showed that addition of hz1613F12-24 DAR4 induces high cell cytotoxicity in different cell lines. No cytotoxicity was measured on MCF-7 which did not express Axl. The highest cytotoxic activity was observed for both human tumor cell lines exhibiting the highest cell-surface Axl level of expression. Inversely no significant cytotoxic activity was observed for MCF7 and NCI-H125 human tumor cell lines, exhibiting 71 and 5540 ABC, respectively.

10.2. In Vitro Cytotoxic Activity of hz1613F12-24 DAR2 on a Panel of Human Tumor Cell Lines.

A batch of the hz1613F12-24 DAR2 ADC was also prepared as described above in Example 8 and assessed using an in vitro SN12C cytotoxicity assay as described in 11.1, except that antibody drug conjugate incubation can last 3 or 6 days.

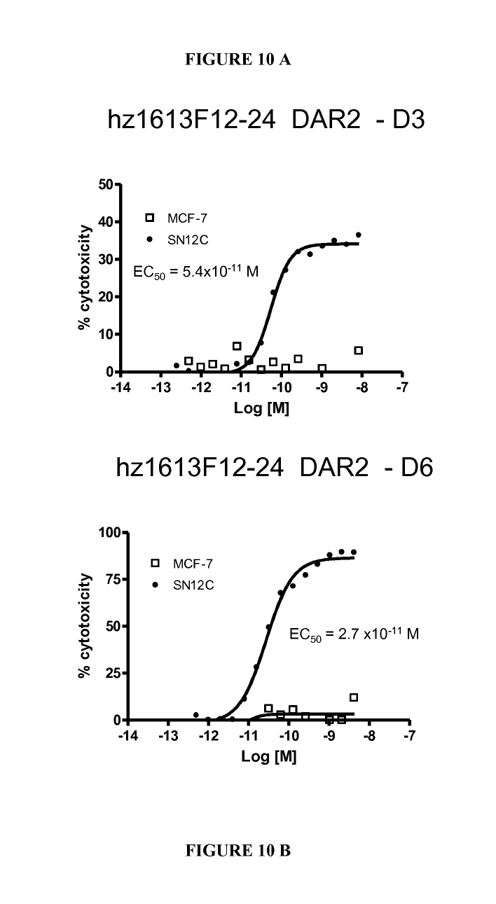

Cytotoxicity curves for both conditions are shown with FIG. 10A corresponding to day 3 and FIG. 10B corresponding to day 6.

Referring to FIGS. 10A and 10B, addition of hz1613F12-24 DAR2 induces high cell cytotoxicity on SN12C cells but not on MCF-7 which does not express Axl. Almost 90% of SN12C cells died in presence of hz1613F12-24 DAR2 after 6 days of culture. The values of the EC₅₀ concentration determined using Prism application with the regression analysis for each curve were of 5.4 10⁻¹¹ M and of 2.7 10⁻¹¹ M after a 3- or a 6-day incubation period with the antibody drug conjugate, respectively.

10.3. In Vitro Cytotoxic Activity of hz1613F12-33 DAR4 on Human Tumor Cell Lines.

The hz1613F12 was also coupled to another linked PBD, varying by the nature of the linker, such as the Drug Moiety 33. This Drug Moiety 33 comprises a PEGylated (n=8) maleimidyl peptide (Val-Cit) linker (presentation in example 8). Once the hz1613F12 was coupled to the Drug Moiety 33, the cytotoxic activity of the resulting hz1613F12-33 DAR4 (preparation described in example 8) was assessed in in vitro cellular assays as described bellow. The ADC was tested against human tumor cell lines expressing various levels of cell-surface Axl as well as against a control Axl⁻ cell line, MCF7.

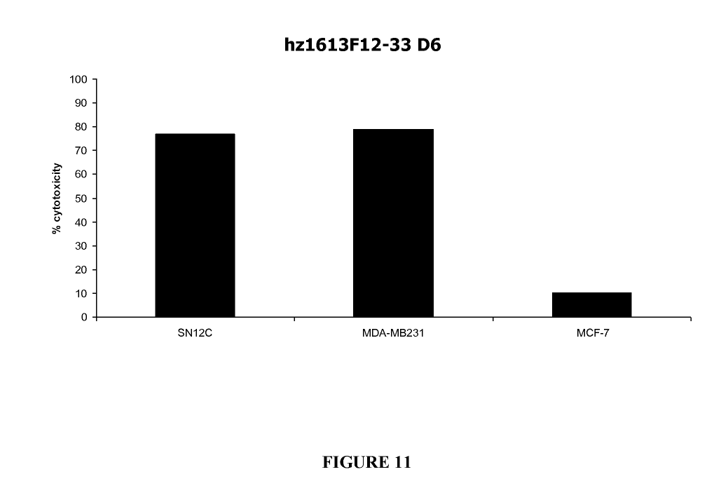

Briefly, human tumor cells were plated for 24 hours in complete culture medium in mw96 plates. The day after, hz1612F12-33 DAR4 was added to the human tumor cells (SN12C, MDAMB231 and MCF7) at a unique concentration of 1 μg/ml. Triplicate wells were prepared for each condition. Following the addition of the antibody drug conjugate, cells were incubated for 6 days at 37° C. Cell viability was assessed using CellTiter-Glo® Luminescent Cell Viability Assay (Promega; Madison; USA) according to manufacturer's protocol. Percentage of cytotoxicity was determined at a 1 μg/ml concentration of the antibody drug conjugate at day 6 (FIG. 11).

Data in FIG. 11 showed that the percentages of the cytotoxicity activity observed on the human tumor cells after a 6-day incubation period with hz1613F12-33 DAR4. Thus hz1613F12-33 DAR4 induced 77% and 79% cytotoxicity of SN12C and MDA-MD231 cells, respectively. In these experimental conditions, the measured cytotoxicity on MCF-7 cells which did not express Axl, was ˜10%.

### Example 11

### Effect of Humanized Forms hz1613F12-24 DAR2 on Human Tumor Cell Xenograft Models in Mice

11.1. In Vivo Anti-Tumoral Activity of Various Humanized Forms of the hz1613F12-24 DAR2 ADC in SN12C Xenograft Model in Mice.

Once coupled to the Drug Moiety 24, several humanized forms of the 1613F12 antibody are selected for in vivo SN12C xenograft model in mice.

All animal procedures were performed according to the guidelines of the 2010/63/UE Directive on the protection of animals used for scientific purposes. The protocol was approved by the Animal Ethical Committee of the Pierre Fabre Institute.

For the SN12C xenograft experiments, athymic 7-week-old female nude mice (Harlan, France) were housed in a light/dark cycle of 12/12 h and fed with sterilized rodent diet and water ad libitum.

SN12C cells from NCI-Frederick Cancer were routinely cultured in RPMI 1640 medium (Lonza), 10% FCS (Sigma), 1% L-Glutamine (Invitrogen). Cells were split 48 hours before engraftment so that they were in exponential phase of growth. Seven million SN12C cells were subcutaneously engrafted in PBS to 7 weeks old female Athymic nude mice. Around twenty days after implantation, when tumors reached an average size of 115-130 mm³, the animals were divided into groups of 6 mice according to tumor size and aspect. The different treatments are then applied. The health status of animals was monitored daily. Tumor volume was measured twice a week with an electronic calliper until study end. Tumor volume is calculated with the following formula: p/6×length×width×height. Toxicity was evaluated following the weight of the animals three times per week. Statistical analyses were performed at each measure using a Mann-Whitney test.

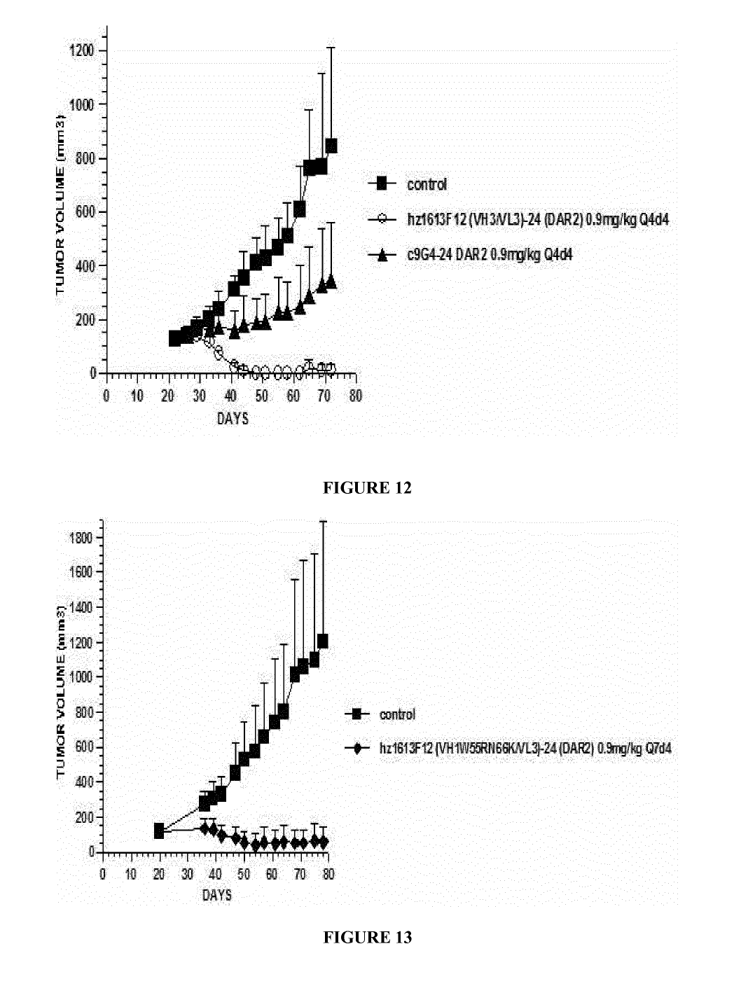

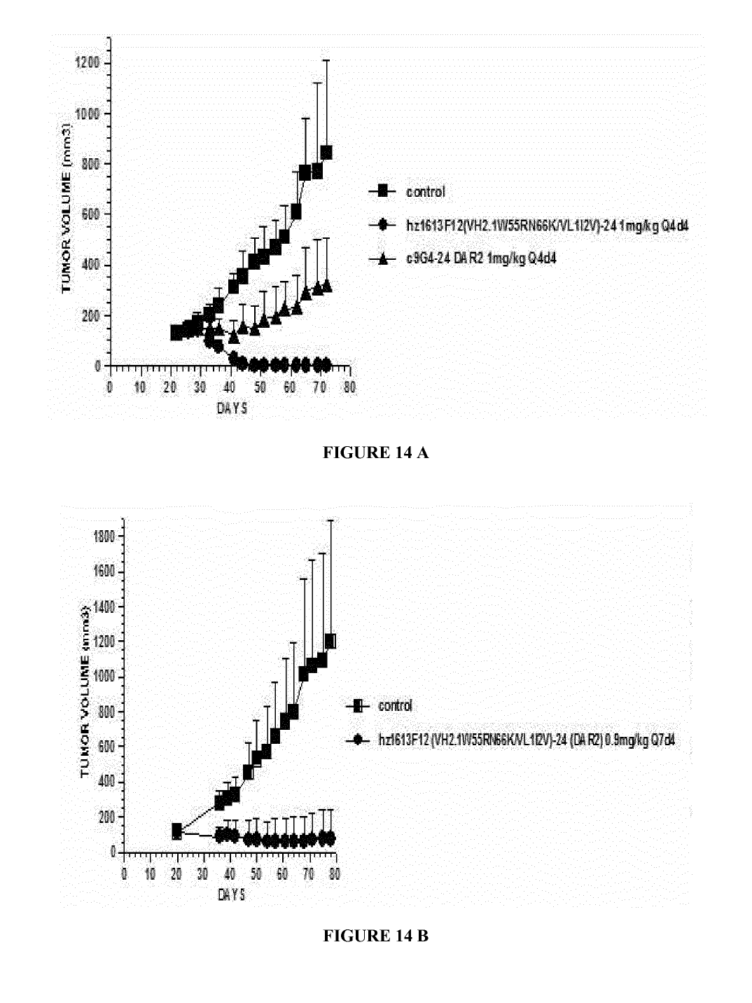

In the present example, the anti-tumoral activities of three distinct humanized forms of the 1613F12 antibody coupled at DAR 2 to the drug moiety 24 are presented: hz1613F12 (VH3/VL3)-24 in FIG. 12, hz1613F12(VH1W55RN66K/VL3)-24 in FIG. 13 and hz1613F12 (VH2.1W55RN66K/VL1I2V)-24 in FIGS. 14A-14B. Several doses and schedules of administration are also documented.

FIG. 12 shows that a strong anti-tumoral effect of the hz1613F12 (VH3NL3)-24 ADC in the SN12C xenograft model. Complete regressions are observed for all the hz1613F12 (VH3/VL3)-24 DAR2 treated animals from D48. Statistical analyses of the measures give a P value bellow 0.02 between D36 and D72 when compared tumor reduction of the hz1613F12 (VH3/VL3)-24 treated animals with that of c9G4-24 treated animals. V³ at D22: 126 mm³; CR 5/5 from D48 to D65.

FIG. 13 illustrates that the hz1613F12 (VH1W55RN66K/VL3)-24 ADC triggers potent anti-tumoral activity against human SN12C renal cells. Complete regression of the SN12C tumor is observed in 3 animals out of 5 since D54. V³ at D20: 115 mm³.

FIGS. 14A-14B present the anti-tumoral activity of the hz1613F12 (VH2.1W55RN66K/VL1I2V)-24 ADC in SN12C xenograft model when injected at both 1 mg/kg Q4d4 and 0.9 mg/kg Q7d4. It shows that both schedules of administration are effective to trigger complete regression of all the SN12C tumor treated with the hz1613F12 (VH2.1W55RN66K/VL1I2V)-24 ADC, in opposite to what is observed with the c9G4-24 immunoconjugate. Statistical analysis of the measures from hz1613F12 (VH2.1W55RN66K/VL1I2V)-24 and c9G4-24 treated groups at the dose of 1 mg/kg Q4d4 give P values bellow 0.05 between D29 and D72.

For FIG. 14A, V³ at D22: 126 mm³ and CR 5/5 since D48.

For FIG. 14B, V³ at D20: 115 mm³ and CR 4/5 since D61.

In these experiments, no toxicity nor mortality is observed during treatment.

11.2. In Vivo Anti-Tumoral Activity of hz1613F12-24 ADC in NCI-H1299, PANC-1 and MDA-MB-231 Xenograft Model in Mice.

In order to further document the potential future clinical indications, the hz1613F12 (VH3/VL3)-24 ADC was injected to different xenograft models. Three of them are described in the present example using different human cells: the NCI-H1299 non-small cell lung carcinoma cell line, the PANC-1 pancreatic cancer cells and the MDA-MB-231 breast cancer cells (which are triple-negative (ER-, PR-, no HER2 overexpression)).

In order to graft cells subcutaneously into mice, cells were split 48 hours before engraftment so that they are in exponential phase of growth.

Specific experimental conditions are applied for each cell line. First, NCI-H1299 cells from the ATCC were routinely cultured in RPMI 1640 medium (Lonza) 10% SVF (Sigma), 1% L-glutamine (Invitrogen). Seven million NCI-H1299 cells were engrafted in PBS in 7 weeks old female SCID mice. Around twenty six days after engraftment, when tumors reached an average size of 130-170 mm³, the animals were divided into groups of 5 mice according the tumor size and aspect. Secondly, PANC-1 cells from the ATCC were routinely cultured in DMEM medium (Lonza), 10% SVF (Sigma). Seven million PANC-1 cells were engrafted in PBS in 7 weeks old female athymic nude mice. Around twenty seven days after engraftment, when tumors reached an average size of 140-170 mm³, the animals were divided into groups of 6 mice according the tumor size and aspect. Thirdly, MDA-MB-231 cells from the ATCC were routinely cultured in DMEM medium (Lonza), 10% SVF (Sigma). Ten million MDA-MB-231 cells were engrafted in PBS in 7 weeks old female NOD/SCID mice. Around twenty days after engraftment, when tumors reached an average size of 145-165 mm³, the animals were divided into groups of 6 mice according the tumor size and aspect.

Then different schedules of treatments are applied and the health status of animals was monitored daily. Tumor volume was measured twice a week with an electronic calliper until study end. Tumor volume was calculated with the following formula: π/6×length×width×height. Toxicity was evaluated following the weight of animals three times per week. Statistical analyses were performed at each measure using a Mann-Whitney test.

In these different models, the hz1613F12 (VH3/VL3)-24 ADC was administrated once i.p. at the dose of 5 mg/kg. In parallel the capped-drug moiety 24 is injected at the equivalent dose of that corresponding to 5 mk/kg of hz1613F12 (VH3/VL3)-24 DAR2. More precisely, drug moiety 24 was capped by N-acetyl cysteine under the following conditions. A 10 mM stock solution of compound 24 was diluted to 0.25 mM in 10 mM borate buffer pH 8.4 containing 150 mM NaCl, 2 mM EDTA and 25% DMSO. A 2.6 molar excess of N-acetyl cysteine was added from a 10 mM solution in 10 mM borate buffer pH 8.4 containing 150 mM NaCl and 2 mM EDTA. The reaction was carried out at room temperature for 45 minutes. After incubation, the capped compound 24 was diluted in 25 mM His buffer pH 6.5 containing 150 mM NaCl before sterile filtration and storage at 4° C. Capping was controlled by LC-MS analysis.

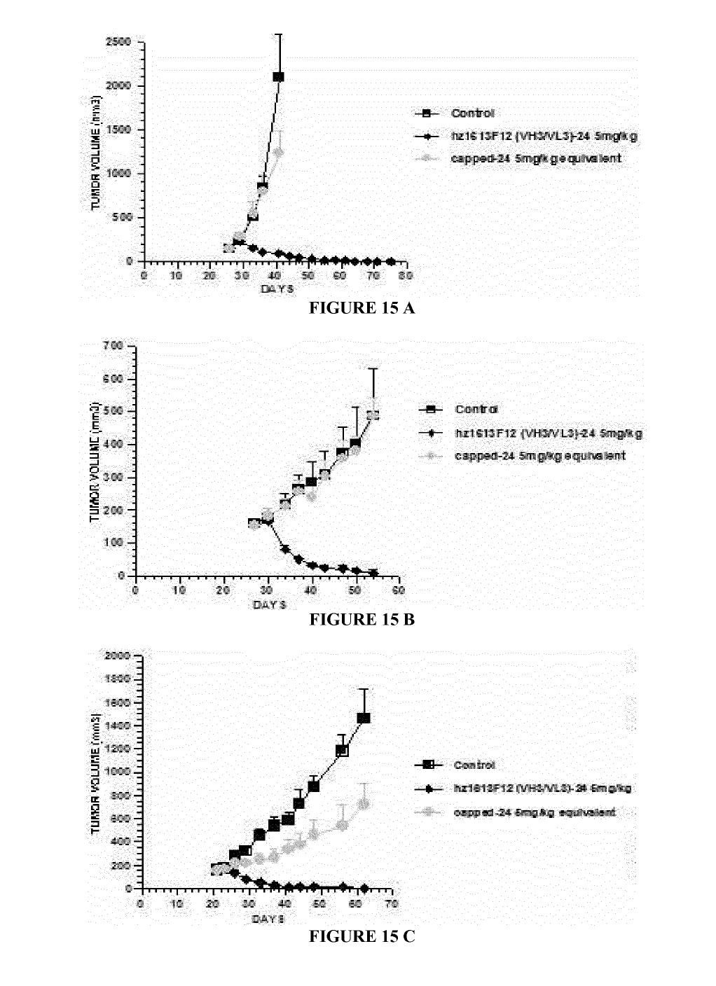

Data are presented in FIG. 15.

Table 6 for FIG. 15A

| | D26 | D29 | D33 | D36 | D41 |
|---|---|---|---|---|---|
| Control/hz1613F12 (VH3/VL3)-24 | 0.222 | 0.548 | 0.008 | 0.008 | 0.008 |
| 5 mg/kg | | | | | |
| Control/capped-24 5 mg/kg | 0.548 | 0.310 | 0.842 | 0.548 | 0.008 |
| equivalent | | | | | |

TABLE 7
FIG. 15B

| | D 27 | D 30 | D 34 | D 37 | D 40 | D 43 | D 47 | D 50 | D 54 |
|---|---|---|---|---|---|---|---|---|---|
| Control/hz1613F12 (VH3/VL3)-24 5 mg/kg | 0.132 | 0.394 | 0.002 | 0.002 | 0.002 | 0.002 | 0.002 | 0.002 | 0.002 |
| Control/capped-24 5 mg/kg equivalent | 0.484 | 0.394 | 0.700 | 1.000 | 0.132 | 0.484 | 0.394 | 0.938 | 0.700 |

TABLE 8
FIG. 15C

| | D 21 | D 23 | D 26 | D 29 | D 33 | D 37 | D 41 | D 44 | D 48 | D 56 | D 62 | D 65 |
|---|---|---|---|---|---|---|---|---|---|---|---|---|
| hz1613F12 (VH3/VL3)-24 5 mg/kg versus | 0.818 | 0.484 | 0.002 | 0.002 | 0.002 | 0.002 | 0.002 | 0.002 | 0.002 | 0.002 | 0.002 | 0.002 |
| capped-24 5 mg/kg SG3249 equivalent | | | | | | | | | | | | |
| Control versus hz1613F12(VH3/VL3)-24 5 mg/kg | 0.310 | 0.818 | 0.002 | 0.002 | 0.002 | 0.002 | 0.002 | 0.002 | 0.002 | 0.002 | 0.002 | 0.002 |
| Control versus capped-24 5 mg/kg SG3249 equivalent | 0.180 | 0.700 | 0.004 | 0.002 | 0.002 | 0.002 | 0.002 | 0.002 | 0.002 | 0.002 | 0.002 | 0.002 |

Data obtained in NCI-H1299 xenograft model show a 95.7% of growth inhibition at D41. At D75, 4 mice out of 5 treated with the hz1613F12 (VH3/VL3)-24 at 5 mg/kg, present complete regression of the NCH-H1299 tumor.

Similarly, a 98.4% of growth inhibition of the PANC1 tumor is observed at D54. In addition, at D54 in the hz1613F12 (VH3/VL3)-24 treated group at the dose of 5 mg/kg, 2 mice out of 6 present complete regression and 2 mice out of 6 have no measurable tumor. In both experiment, no effect of the capped-24 compound is observed. Finally, all (6 out of 6) the animals treated with hz1613F12 (VH3/VL3)-24 ADC present complete tumor regression at D62

This example illustrates the potency of the hz1613F12-24 ADC to induce regression of Axl expressing tumor cells.

### Example 12

### Effect of the hz1613F12-24 DAR2 ADC in A549 Orthotopic Model

In the present example, the hz1613F12-24 DAR2 ADC is evaluated in a metastatic model of human non-small cell lung carcinoma (NSCLC), the A549 adenocarcinoma, by inoculating tumor cells into the pleural space of nude mice. The intrathoracically implantation of the tumor leads to an increased tumorigenicity and metastatic potential as compared to the s.c. xenograft model and thus could be more relevant to the clinical situation.

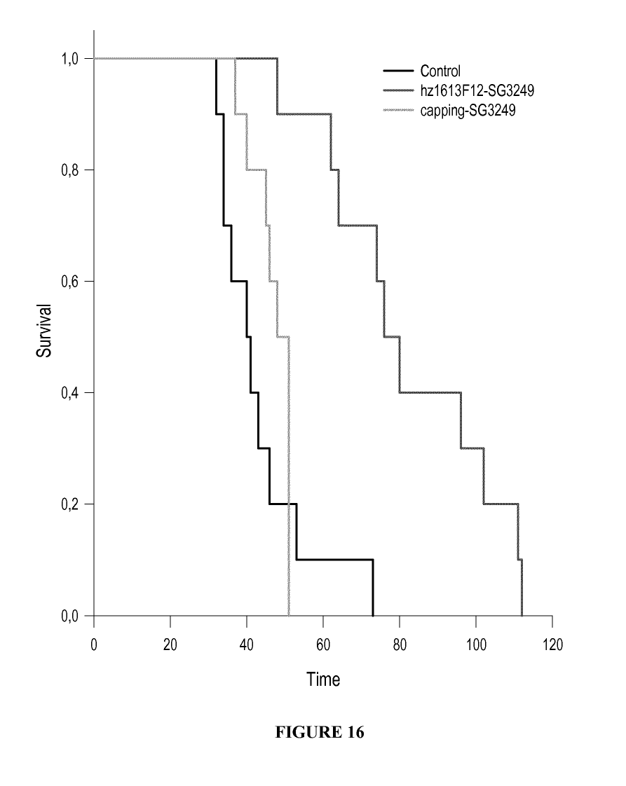

More precisely, the orthotopic model is set up for A549 human lung tumor cells as described by Kraus-Berthier et al. Briefly, animals are anesthetized with a 4/1 mixture of ketamine (Imalgene® 500; Rhone Merieux, Lyon, France) and xylasine (Rompun® at 2%; Bayer, Puteaux, France) administered i.p. One million tumor cells were implanted through the chest wall into the left pleural space of nude mice (i.pl.) in a volume of 100 μl using a 26-gauge needle. The primary tumor had on day 4 already spread locally to continuous structures, including mediastinum, lung and diaphragm. To better mimic a clinical situation, treatment started only when the disease was developed, 7 days after i.p. injection of A549 tumor cells. Groups of 10 mice were generated at random and treated once 14 days post-cell implantation at a dose of 7 mg/kg for hz1613F12 (VH3/VL3)-24 DAR2 and 7 mg/kg drug equivalent for capped-24. Control mice received the vehicle. Mice were monitored for changes in body weight and life span. The antitumor activity was evaluated as follows: T/C %=median survival time of treated group/median survival time of control group×100. Log-Rank Test statistical analysis were performed using SigmaStat software. The significance threshold was 5%. Data are presented in FIG. 16.

TABLE 9

| Log-Rank Test: | Statistic | |
|---|---|---|
| Comparisons | P Value | Significant? |
| Control vs. hz1613F 12-24 | 0.0000550 | Yes |
| Hz 1613F 12-24 vs. capped-24 | 0.000156 | Yes |
| Control vs. capped-24 | 0.343 | No |

As presented in FIG. 16, when evaluated in human A549 orthotopic model, the hz1613F12-24 DAR2 ADC given i.p. at a dose of 7 mg/kg demonstrated a marked antitumor activity against human A549 carcinomas. In this human lung cancer model, the hz1613F12-24 DAR2 ADC triggered a significant survival benefit for the animals treated with hz1613F12-24 DAR2 versus control groups (PBS, capped-24). T/C values are respectively of about 193% and 158%.

### ABBREVIATIONS

Ac acetyl
Acm acetamidomethyl
Alloc allyloxycarbonyl
Boc di-tert-butyl dicarbonate
t-Bu tert-butyl
Bzl benzyl, where Bzl-OMe is methoxybenzyl and Bzl-Me is methylbenzene
Cbz or Z benzyloxy-carbonyl, where Z—Cl and Z—Br are chloro- and bromobenzyloxy carbonyl respectively

### DMF N,N-dimethylformamide

Dnp dinitrophenyl
DTT dithiothreitol
Fmoc 9H-fluoren-9-ylmethoxycarbonyl
imp N-10 imine protecting group: 3-(2-methoxyethoxy)propanoate-Val-Ala-PAB MC-OSumaleimidocaproyl-O—N-succinimide
Moc methoxycarbonyl
MP maleimidopropanamide
Mtr 4-methoxy-2,3,6-trimethtylbenzenesulfonyl
PAB para-aminobenzyloxycarbonyl
PEG ethyleneoxy
PNZ p-nitrobenzyl carbamate
Psec 2-(phenylsulfonyl)ethoxycarbonyl
TBDMS tert-butyldimethylsilyl
TBDPS tert-butyldiphenylsilyl
Teoc 2-(trimethylsilyl)ethoxycarbonyl
Tos tosyl
Troc 2,2,2-trichlorethoxycarbonyl chloride
Trt trityl
Xan xanthyl

1. An antibody-drug conjugate having the structural general formula (I):
CBA-(D)ₙ (I)
wherein CBA is an antibody consisting of 1613F12, or an antigen binding fragment thereof, comprising the three light chain CDRs of sequences SEQ ID No. 1, 2 and 3 and the three heavy chain CDRs of sequences SEQ ID No. 4, 5 and 6; n is 1 to 12; and D is a drug consisting of a pyrrolobenzodiazepine dimer (PBD dimer) having the formulae (AB) or (AC) 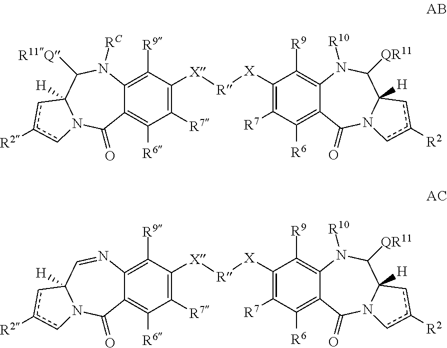
wherein:
the dotted lines indicate the optional presence of a double bond between C1 and C2 or C2 and C3;
R² is independently selected from H, OH, ═O, ═CH₂, CN, R, OR, ═CH—R^{D}, ═C(R^{D})₂, O—SO₂—R, CO₂R and COR, and optionally further selected from halo or dihalo;
where R^{D} is independently selected from R, CO₂R, COR, CHO, CO₂H, and halo;
R⁶ and R⁹ are independently selected from H, R, OH, OR, SH, SR, NH₂, NHR, NRR′, NO₂, Me₃Sn and halo;
R⁷ is independently selected from H, R, OH, OR, SH, SR, NH₂, NHR, NRR′, NO₂, Me₃Sn and halo;
R¹⁰ is a linker connected to CBA;
Q is independently selected from O, S and NH;
R¹¹ is either H, or R or, where Q is O, SO₃M, where M is a metal cation;
R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;
X is O, S or NH;
R″ is a C₃₋₁₂ alkylene group, which chain may be interrupted by one or more heteroatoms, e.g. O, S, N(H), NMe and/or aromatic rings, e.g. benzene or pyridine, which rings are optionally substituted; and
wherein R^{2″}, R^{6″}, R^{7″}, R^{9″}, X″, Q″ and R^{11″} and are as defined according to R², R⁶, R⁷, R⁹, X, Q and R¹¹ respectively, and R^{C} is a capping group.

2. The antibody-drug conjugate of claim 1, wherein 1613F12 is humanized.

3. The antibody-drug conjugate of claim 1 or 2, wherein 1613F12, or an antigen binding fragment thereof, comprises a light chain variable domain of sequence SEQ ID No. 17 or any sequence exhibiting at least 80% identity with SEQ ID No. 17.

4. The antibody-drug conjugate of claim 3, wherein 1613F12, or an antigen binding fragment thereof, comprises a light chain variable domain selected from sequences SEQ ID No. 18 to 28 or any sequence exhibiting at least 80% identity with SEQ ID No. 18 to 28.

5. The antibody-drug conjugate of claim 1 or 2, wherein 1613F12, or an antigen binding fragment thereof, comprises a heavy chain variable domain of sequence SEQ ID No. 29 or any sequence exhibiting at least 80% identity with SEQ ID No. 29.

6. The antibody-drug conjugate of claim 5, wherein 1613F12, or an antigen binding fragment thereof, comprises a heavy chain variable domain selected from sequences SEQ ID No. 30 to 49 or any sequence exhibiting at least 80% identity with SEQ ID No. 30 to 49.

7. The antibody-drug conjugate of claim 1 or 2, wherein 1613F12, or an antigen binding fragment thereof, comprises a light chain variable domain of sequence SEQ ID No. 81 or any sequence exhibiting at least 80% identity with SEQ ID No. 81, and a heavy chain variable domain of sequence SEQ ID No. 82 or any sequence exhibiting at least 80% identity with SEQ ID No. 82.

8. The antibody-drug conjugate of claim 7, wherein 1613F12 is selected from antibodies, or antigen binding fragments thereof, comprising:
a) a light chain variable domain of sequence SEQ ID No. 19 or any sequence exhibiting at least 80% identity with SEQ ID No. 19, and a heavy chain variable domain of sequence SEQ ID No. 40 or any sequence exhibiting at least 80% identity with SEQ ID No. 40;
b) a light chain variable domain of sequence SEQ ID No. 21 or any sequence exhibiting at least 80% identity with SEQ ID No. 21, and a heavy chain variable domain of sequence SEQ ID No. 40 or any sequence exhibiting at least 80% identity with SEQ ID No. 40;
c) a light chain variable domain of sequence SEQ ID No. 27 or any sequence exhibiting at least 80% identity with SEQ ID No. 27, and a heavy chain variable domain of sequence SEQ ID No. 32 or any sequence exhibiting at least 80% identity with SEQ ID No. 32; or
d) a light chain variable domain of sequence SEQ ID No. 28 or any sequence exhibiting at least 80% identity with SEQ ID No. 28, and a heavy chain variable domain of sequence SEQ ID No. 32 or any sequence exhibiting at least 80% identity with SEQ ID No. 32.

9. The antibody-drug conjugate of any of the preceding claims, wherein R¹⁰ is: 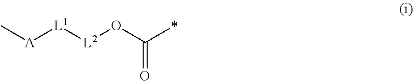
wherein A is a connecting group connecting L¹ to CBA, L¹ is a cleavable linker, L² is a covalent bond or together with —OC(═O)— forms a self-immolative linker, and the asterisk indicates the point of attachment to the N10 position of D.

10. The antibody-drug conjugate of claim 9, wherein A is selected from: 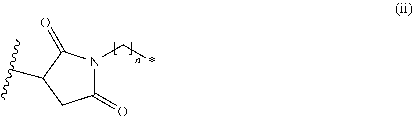
wherein the asterisk indicates the point of attachment to L¹, the wavy line indicates the point of attachment to CBA, and n is 0 to 6;
or 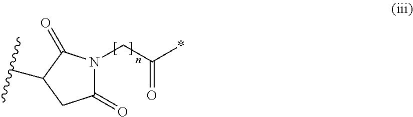
wherein the asterisk indicates the point of attachment to L¹, the wavy line indicates the point of attachment to CBA, and n is 0 to 6;
or 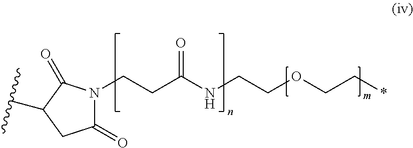
wherein the asterisk indicates the point of attachment to L¹, the wavy line indicates the point of attachment to CBA, n is 0 or 1, and m is 0 to 30;
or 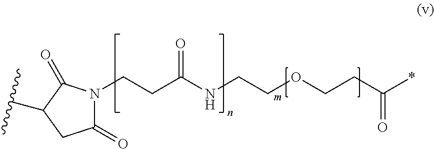
wherein the asterisk indicates the point of attachment to L¹, the wavy line indicates the point of attachment to CBA, n is 0 or 1, and m is 0 to 30.

11. The antibody-drug conjugate of claim 10, wherein CBA is connected to A through a thioether bond formed from a cysteine thiol residue of CBA and a malemide group of A.

12. The antibody-drug conjugate of claim 9, wherein L₁ comprises a dipeptide —NH—X₁—X₂—CO— wherein the group —X₁—X₂— is selected from -Phe-Lys-, -Val-Ala-, -Val-Lys-, -Ala-Lys-, -Val-Cit-, -Phe-Cit-, -Leu-Cit-, -Ile-Cit-, -Phe-Arg-, -Trp-Cit-,
wherein Cit is citrulline.

13. The antibody-drug conjugate of claim 9, wherein —C(═O)O— and L₂ together form the group: 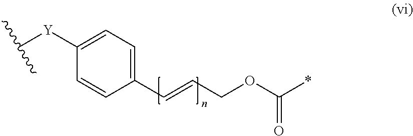
wherein the asterisk indicates the point of attachment to the N10 position of D, the wavy line indicates the point of attachment to the linker L¹, Y is —N(H)—, —O—, —C(═O)N(H)— or —C(═O)O—, and n is 0 to 3.

14. The antibody-drug conjugate of claim 9, wherein L₁ and L₂ together with —C(═O)O— comprise a group selected from: 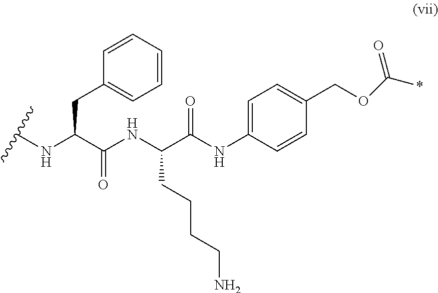 [SMILES: *N[C@@H](Cc1ccccc1)C(=O)N[C@@H](CCCCN)C(=O)Nc1ccc(COC(*)=O)cc1]
wherein the asterisk indicates the point of attachment to the N10 position of D, and the wavy line indicates the point of attachment to the remaining portion of the linker L¹ or the point of attachment to A;
or 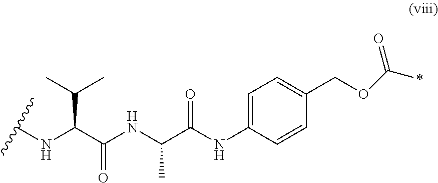 [SMILES: *N[C@H](C(=O)N[C@@H](C)C(=O)Nc1ccc(COC(*)=O)cc1)C(C)C]
wherein the asterisk and the wavy line are as defined above;
or 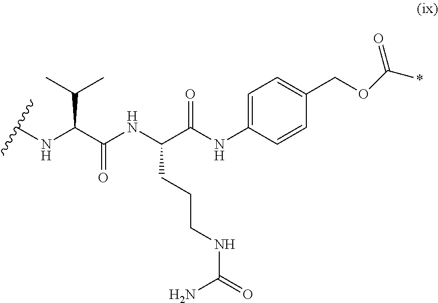 [SMILES: *N[C@H](C(=O)N[C@@H](CCCNC(N)=O)C(=O)Nc1ccc(COC(*)=O)cc1)C(C)C]
wherein the asterisk and the wavy line are as defined above.

15. The antibody-drug conjugate of any of the preceding claims, wherein D is selected from:

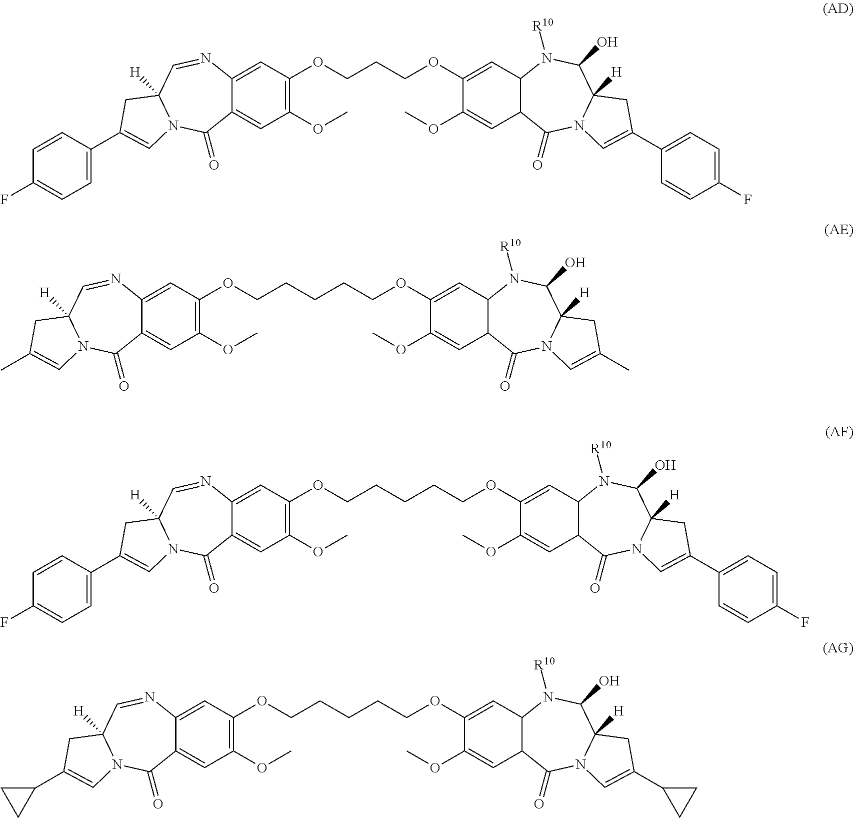

16. An antibody-drug conjugate of the structural general formula selected from: 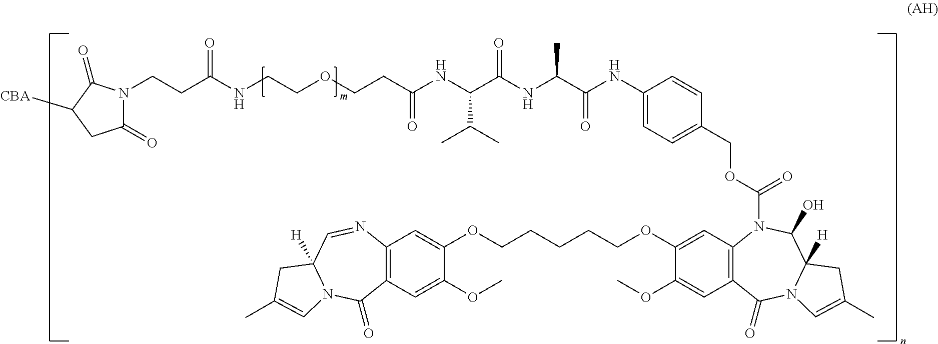
wherein CBA consists of 1613F12, or an antigen binding fragment thereof, m is 0 to 30, and n is 1 to 12;
or 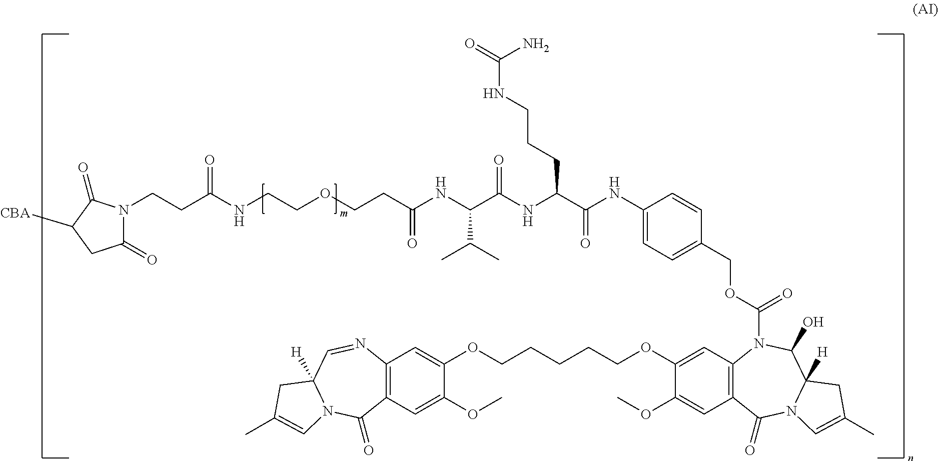
wherein CBA consists of 1613F12, or an antigen binding fragment thereof, m is 0 to 30, and n is 1 to 12.

17. An antibody-drug conjugate having the structural general formula: 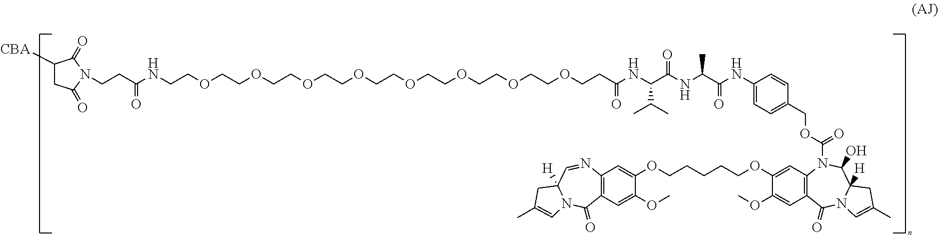
wherein CBA consists of 1613F12, or an antigen binding fragment thereof, and n is 1 to 12;
or 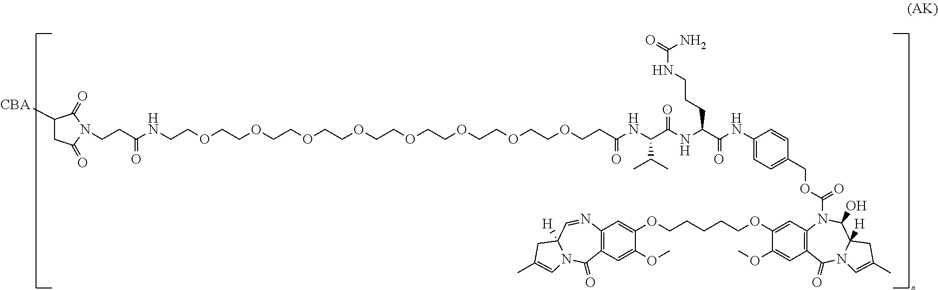
wherein CBA consists of 1613F12, or an antigen binding fragment thereof, and n is 1 to 12.

18. The antibody-drug conjugate of claim 16 or 17, wherein n is 2.

19. The antibody-drug conjugate of claim 16 or 17, wherein n is 4.

20. The antibody-drug conjugate of any of claims 1 to 19 for use in the treatment of an Axl-expressing cancer.

21. A composition comprising at least an antibody-drug conjugate of any of the claims 1 to 19.

22. The composition of claim 21, wherein the composition is a pharmaceutical composition further comprising a pharmaceutically acceptable vehicle.

23. The composition of claim 21 or 22 for use in the treatment of an Axl-expressing cancer.

24. The use of an antibody-drug conjugate of any of claims 1 to 19 or of a composition of any one of claims 21 to 23 for the treatment of an Axl-expressing cancer.

25. The use of claim 24, wherein said Axl-expressing cancer is a cancer chosen from breast, colon, esophageal carcinoma, hepatocellular, gastric, glioma, lung, melanoma, osteosarcoma, ovarian, prostate, rhabdomyosarcoma, renal, thyroid, uterine endometrial cancer, mesothelioma, oral squamous carcinoma and any drug resistant cancer.

26. A method for the treatment of an Axl-expressing cancer in a subject, comprising administering to the subject an effective amount of at least the antibody-drug conjugate of any of claims 1 to 19 or of a composition of claim 21 or 23.

27. A kit comprising at least i) an antibody-drug conjugate of any of claims 1 to 19 and/or a composition of claim 21 or 23 and ii) a syringe or vial or ampoule in which the said antibody-drug conjugate and/or composition is disposed.
